(19) United States
(12) Patent Application Publication
(10) Pub. No.: US 2014/0017227 A1
(43) Pub. Date: Jan. 16, 2014

(54) GENE SIGNATURES FOR USE WITH HEPATOCELLULAR CARCINOMA

(71) Applicants: Suk Peng Chew, Immunos (SG); Alessandra Nardin, Immunos (SG); Jean-Pierre Abastado, Immunos (SG); JinMiao Chen, Immunos (SG); Henry Yang, Immunos (SG)

(72) Inventors: Suk Peng Chew, Immunos (SG); Alessandra Nardin, Immunos (SG); Jean-Pierre Abastado, Immunos (SG); JinMiao Chen, Immunos (SG); Henry Yang, Immunos (SG)

(21) Appl. No.: 13/979,521

(22) PCT Filed: Jan. 13, 2012
(86) PCT No.: PCT/SG2012/000014

§ 371 Date: Sep. 27, 2013

(30) Foreign Application Priority Data
Jan. 14, 2011 (SG) 201100290-4

(51) Int. Cl. C12Q 1/68; G01N 33/574

(52) U.S. Cl. CPC C12Q 1/6886; G01N 33/57484
USPC 424/130.1; 435/6.14; 435/6.11; 435/6.12; 506/9; 506/16; 435/7.1

(57) ABSTRACT

The present invention provides a method for predicting prognosis of hepatocellular carcinoma patients based on measurement of the relative level of expression of a combination of 15 immune genes of interest, or a subset thereof, in the tumors of such patients. Tumor material can come from surgical resection or biopsy. The relative gene expression information may be combined in an algorithm. The signature can be used by itself or in combination with other information such as stage information.

### FIELD OF THE INVENTION

The present invention relates to a method of analyzing hepatocellular carcinoma (HCC) patients using the expression levels of various immune genes in a sample from the tumor, in particular to predict the prognosis of HCC patients. The invention also relates to methods of identifying an agent effective for treating HCC, and also to methods for stratifying HCC.

All documents cited in this text (“herein cited documents”) and all documents cited or referenced in herein cited documents are incorporated by reference in their entirety for all purposes. There is no admission that any of the various documents etc. cited in this text are prior art as to the present invention.

### BACKGROUND

Hepatocellular carcinoma is an aggressive malignancy and claims over 600,000 lives every year worldwide. HCC incidence is rising in Western countries partly due to increased Hepatitis C virus. (HCV) infection. HCC is a heterogeneous disease comprising distinct molecular and clinical subgroups [5-6]. This is largely due to the different HCC etiologies which include hepatitis, alcohol and non-alcohol induced cirrhosis. Geographical and ethnic variations further contribute to its heterogeneity.

There are few treatment options for HCC, in particular for patients with advanced disease where there are limited treatments. Resection remains the treatment choice for many patients but it is also associated with high relapse rate and poor 5-year survival rate. Sorafenib, a tyrosine kinase inhibitor recently approved for advanced HCC, brings only limited improvement in survival [3]. More aggressive treatments, including liver transplantation for suitable patients, improves survival. However, identifying HCC patients likely to benefit from such approaches remains challenging.

With the development of health awareness in the general public, HCC comes to medical attention at earlier stages where often it is hard to determine the prognosis using classical histopathological measurements such as tumor multinodularity and vascular invasion. In the past decade, several laboratories used gene-expression profiling to define the molecular nature and identify prognostic signatures for HCC [8-12]. However, little consensus was reached from such efforts, illustrating the complexity and heterogeneity of this cancer. Each study focused on different molecular pathways and limited attention has so far been given to the tumor immune microenvironment.

### SUMMARY OF THE INVENTION

The current invention describes an immune gene signature derived from resected HCC tumors from Singapore HCC patients (n=61) who are mostly at stage I, for predicting prognosis or survival in HCC patients. The immune gene signature has been validated as being able to predict survival of HCC patients from another region in Asia, Hong Kong (n=56) as well as from Europe, Zurich, Switzerland (n=55); both the Hong Kong and Zurich cohort include more advanced HCC patients—mostly Stage II or III.

In at least some embodiments, the gene signature includes a combination of three to fifteen (and preferably five to fourteen) immune genes out of a total 15 immune genes of interest whose relative expression is preferably analysed in a classifier (algorithm). Overall, an increase in mRNA expression of these genes is associated with better prognosis. The predictive power of the combination of any five to fourteen immune genes (the classifier) of these 15 immune genes is stronger than any single individual gene by itself.

This immune signature can be used by itself to analyse HCC patients or with other information, such as staging information. This application describes various uses of the immune signature, in particular in predicting the prognosis of HCC patients (e.g. < > of 5 years survival).

A preferred embodiment of the invention provides a method for predicting prognosis (< > 5 years survival) of hepatocellular carcinoma patients based on measurement of the relative level of expression of a combination of three to fifteen immune genes (and preferably 5 to 14 immune genes) out of 15 immune genes of interest in the tumors of such patients. Tumor material can come from surgical resection or biopsy. The relative gene expression information can optionally be combined in an algorithm.

### GLOSSARY OF TERMS

This section is intended to provide guidance on the interpretation of the words and phrases set forth below (and where appropriate grammatical variants thereof). Further guidance on the interpretation of certain words and phrases as used herein (and where appropriate grammatical variants thereof) may additionally be found in other sections of this specification.

As used herein, the singular form “a,” “an,” and “the” include plural references unless the context clearly dictates otherwise. For example, the term “an agent” includes a plurality of agents, including mixtures thereof and reference to “the nucleic acid sequence” generally includes reference to one or more nucleic acid sequences and equivalents thereof known to those skilled in the art, and so forth.

As used herein, the term “comprising” means “including”. Thus, for example, a gene signature “comprising three genes” may consist exclusively of three genes or may include one or more further marker genes.

The term “stratifying” as used herein refers to describing or separating a patient population into more homogeneous subpopulations according to specified criteria. In one embodiment, patients can be stratified for different treatment protocols (e.g. more or less aggressive treatment, surgical intervention, liver transplantation, immunotherapy, chemotherapy with a given drug or drug combination, and/or radiation therapy). Patients may also be stratified into those having a poor or good prognosis, or those have a short or long predicted survival.

The term “classifying” as used herein refers to the process of determining or arranging patients into a particular group depending on their tumor sample profile. In at least some embodiments, the term “classifying” refers to classifying a patient as having a particular prognosis, e.g. a poor or good prognosis, or short or long predicted survival.

The term “prognosis” as used herein relates to providing a forecast or prediction of the likely course or outcome of HCC. The term includes a reference to predicting HCC progression (e.g. recurrence or metastatic spread), survival, drug resistance, partial or complete remission, or a good or poor outcome (good or poor prognosis respectively). The term also includes predicting the timing of any of the aforementioned (e.g. more than, less than, or equal to a given number of years (e.g. 0.5, 1, 2, 3, 4, 5, 6, 7, 8, 9, 10 or more years)). Thus, for instance, providing a prognosis may comprise predicting the patient's survival as being more than, less than, or equal to a given number of years.

Where survival, recurrence, metastatic spread or another event is described herein in relation to a given period of time, the period of survival may optionally be measured from first diagnosis, first treatment, when the tumor is resected, or from any other convenient or suitable time point. Preferably, survival is measured from when the tumour is resected.

Where survival, recurrence of metastatic spread or another event is described herein in relation to a given period of time (e.g. as being more than, less than, equal to, or within etc. a given time period), the given period of time is preferably a time point which is within one of the following ranges: 0 to 18 years, 0 to 17 years, 0 to 16 years, 0 to 15 years, 0 to 14 years, 0 to 13 years, 0 to 12 years, 0 to 11 years, 0 to 10 years, 0 to 9 years, 0 to 8 years, 0 to 7 years, 0 to 6 years, 0 to 5 years, 0 to 4 years, 0 to 3 years, 0 to 2 years, 0 to 1 years, 1 to 18 years, 1 to 16 years, 1 to 14 years, 1 to 12 years, 1 to 10 years, 1 to 9 years, 1 to 8 years, 1 to 7 years, 1 to 6 years, 1 to 5 years, 1 to 4 years, 1 to 3 years, 1 to 2 years, 2 to 18 years, 2 to 16 years, 2 to 14 years, 2 to 12 years, 2 to 10 years, 2 to 9 years, 2 to 8 years, 2 to 7 years, 2 to 6 years, 2 to 5 years, 2 to 4 years, 2 to 3 years, 3 to 18 years, 3 to 17 years, 3 to 16 years, 3 to 15 years, 3 to 14 years, 3 to 13 years, 3 to 12 years, 3 to 11 years, 3 to 10 years, 3 to 9 years, 3 to 8 years, 3 to 7 years, 3 to 6 years, 3 to 5 years, 3 to 4 years, 4 to 10 years, 4 to 9 years, 4 to 8 years, 4 to 7 years, 4 to 6 years, or 4 to 5 years. The aforementioned ranges are inclusive and so it will be understood that a time point which is within the range of 4 to 5 years, for example, is to be understood as including both endpoints of the range so that the time point may, for example be 4 or 5 years (or any time point falling between these endpoints, such as 4.5 years). Accordingly, in at least some embodiments providing a prognosis may comprise predicting the patient's survival as being: (i) more than, less than, or equal to 4 years; or (ii) more than, less than, or equal to 5 years. Other preferred cut-offs for survival include 3 and 6 years. Thus, in some embodiments of the invention the methods comprise predicting the patient's survival as being more than, less than or equal to 3 or 6 years.

The term “poor prognosis” as used herein refers to where an undesired outcome (“poor outcome”) is predicted for the HCC. Examples of poor outcomes include reappearance of the HCC after treatment (optionally within a given time period, such as within 1, 2, 3, 4, 5, 6, 7, 8, 9, 10 or more years); the reoccurrence of metastases (optionally within a given time period such as within 1, 2, 3, 4, 5, 6, 7, 8, 9, 10 or more years); or survival for less than a given period of time, e.g. less than 0.5, 1, 2, 3, 4, 5, 6, 7, 8, 9 or 10 years survival.

In at least some embodiments of the invention, the term “poor prognosis” as used herein refers to: (i) predicted survival for less than a time point which falls within, or is equal to, 3 to 6 years (e.g. survival for less than 3, 4, 5 or 6 years), with the survival preferably being measured from the time of tumor resection); (ii) when the gene expression profile has a higher similarity to a poor prognosis template than to a good prognosis template; (iii) when the gene expression profile is similar to a poor prognosis template and/or dissimilar to a good prognosis template; or (iv) predicted survival is less than the mean, mode or median of the number of years survival of a HCC patient cohort.

By a “patient cohort” we refer to a population of HCC patients, e.g. a population of at least 5, 10, 15, 20, 25, 30, 35, 40, 45, 50, 55, 50, 55, 60, 65, 70, 75, 85 or 95 patients. The patients of a particular cohort may be restricted geographically such as to patients in a particular city or country at the time of first diagnosis or resection (see e.g. the Singapore training cohort).

When SVM and/or KNN algorithms are used the term “poor prognosis” preferably refers to less than 5-years survival for a HCC patient. Preferably, when NTP algorithm is used, a patient is classified as having a “poor prognosis” when the patient's gene expression profile is more similar to the poor prognosis template than to the good prognosis template, both calculated using the NTP algorithm. It should be noted that NTP does not have a cut-off survival year. This is explained in more detail in Algorithm 3.

The term “good prognosis” as used herein refers to where a desired outcome (“good outcome”) is predicted for the HCC. Examples of good outcomes include partial or complete remission; the non-reoccurrence of metastases, optionally within a given period of time e.g. 0.5, 1, 2, 3, 4, 5, 6, 7, 8, 9, 10, 11, 12, 13, 14, 15, 16, 17, 18 or more years; or survival for a given period of time, e.g. more than or equal to 0.5, 1, 2, 3, 4, 5, 6, 7, 8, 9, 10, 11, 12, 13, 14, 15, 16, 17 or 18 years survival.

In at least some embodiments of the invention, the term “good prognosis” refers to: (i) predicted survival of more than or equal to a time point which falls within, or is equal to, 3, 4, 5 or 6 years, with the survival preferably being measured from the time of tumor resection; (ii) when the gene expression profile has a higher similarity to a good prognosis template than to a poor prognosis template; (iii) when the gene expression profile is similar to a good prognosis template and/or dissimilar to a poor prognosis template; or (iv) predicted survival is less than the mean, mode or median of the number of years survival of a HCC patient cohort.

When SVM and/or KNN algorithms are used the term “good prognosis” preferably refers to more than or equal to 5-years survival for a HCC patient. Preferably, when NTP algorithm is used, a patient is classified as having a “good prognosis” when his gene expression profile is more similar to the good prognosis template than to the poor prognosis template, both calculated using the NTP algorithm. It should be noted that NTP does not have a cut-off survival year. This is explained in more detail in Algorithm 3.

The terms “treatment”, “therapeutic intervention” and “therapy” may be used interchangeably herein (unless the context indicates otherwise) and these terms refer to both therapeutic treatment and prophylactic or preventative measures, wherein the object is to try and prevent or slow down (lessen) the targeted pathologic condition or disorder. In tumor treatment, the treatment may directly decrease the pathology of tumor cells, or render the tumor cells more susceptible to treatment by other therapeutic agents, e.g., radiation and/or chemotherapy. The aim or result of tumor treatment may include, for example, one or more of the following: (1) inhibition (i.e., reduction, slowing down or complete stopping) of tumor growth; (2) reduction or elimination of symptoms or tumor cells; (3) reduction in tumor size; (4) inhibition of tumor cell infiltration into adjacent peripheral organs and/or tissues; (5) inhibition of metastasis; (6) enhancement of anti-tumor immune response, which may, but does not have to, result in tumor regression or rejection; (7) increased survival time; and (8) decreased mortality at a given point of time following treatment. Treatment may entail treatment with a single agent or with a combination (more than two) of agents. Treatment may optionally comprise a course of treatment.

An “agent” is used herein broadly to refer to, for example, a drug/compound or other means for treatment, e.g. radiation treatment or surgery. Examples of treatment include surgical intervention, liver transplantation, immunotherapy, chemotherapy with a given drug or drug combination, radiation therapy, neoadjuvant treatment, diet, vitamin therapy, hormone therapies, gene therapy, cell therapy, antibody therapy etc. The term “treatment” also includes experimental treatment e.g. during drug screening or clinical trials.

The phrase “predicting the efficacy of a therapeutic intervention” includes predicting whether the patient responds favourably or unfavourably to treatment and/or the extent of those responses.

The phrase “evaluating the efficacy of a therapeutic intervention” includes assessing whether the patient responds favourably or unfavourably to treatment and/or the extent of those responses.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DESCRIPTION OF THE INVENTION

The present invention relates to methods for analyzing a patient with HCC. More specifically, the invention provides methods which comprise: (a) determining the expression levels of three or more genes in a patient-derived tumor sample, wherein the three or more genes are selected from the genes listed in Table 1, 2A, 2B, 3, 4, 14, 15 and/or 16 (see Tables below); and (b) using the gene expression level information to analyze the patient, for instance to extrapolate prognostic information of the patient. By “analyzing a patient” we include classifying or stratifying the patient, providing a patient prognosis (e.g. poor or good; long or short survival), monitoring disease progression, predicting efficacy of a therapeutic intervention, selecting treatment for the patient, and evaluating the efficacy of a therapeutic intervention. The genes listed in Table 1, 2A, 2B, 3, 4, 14, 15 and 16 are herein referred to as the “immune genes of the invention”. Optionally, the gene expression level information is analyzed using one or more of: at least one algorithm, statistical analysis or a computer. The predictive power of the combination of these 15 immune genes of Tables 1, 2A, 2B, 3, 4, 14, 15 and 16 is stronger than any single individual gene by itself.

Depending on prognosis, patients can be stratified for different treatment (e.g. more or less aggressive treatment, surgical intervention, liver transplantation, immunotherapy, chemotherapy with a given drug or drug combination, radiation therapy, neoadjuvant treatment, gene therapy, cell therapy, antibody therapy etc.).

The term “treatment” also includes experimental treatment e.g. during drug screening or clinical trials. The ability to provide a prognosis for HCC patients will help in disease management such as in selection of patients with better prognosis profile for liver transplantation. Advantageously, the immune genes of the invention are predictive of HCC prognosis irrespective of patient ethnicity and disease etiology.

The term HCC as used herein includes all forms of HCC including stage I, II, III and IV HCC. Staging can be performed in accordance with the TNM staging system which is used internationally.

Optionally, the HCC is: (a) stage I; (b) stage II; (c) stage I or II; (d) stage II or III; (e) stage I, II or III; (f) stage II, III or IV; or (g) stage I, II, III or IV. Preferably, the HCC is not stage III. Preferably, the HCC is not stage IV.

The term “patient” as used herein includes human patients and other mammals and includes any individual that is, or has been, afflicted with HCC, or which it is desired to analyse or treat using the methods of the invention. Suitable mammals that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes). Preferably, the patient is a human patient. Where non-human nucleic acid or protein/polypeptides are being assayed the expression level of homologs to the genes set forth in Table 1, 2A, 2B, 3, 4, 14, 15 or 16 may be assayed and references to the immune genes of the invention are to be interpreted to include such homolog sequences. In the present invention, the patient may be male or female. Optionally, the patient may be undergoing treatment, for example experimental treatment, for HCC. In this context, the method would provide a surrogate biomarker for measurement of efficacy of the treatment. The patient may have stage I, II, III or IV HCC. Optionally, the patient is: (a) a stage I or II patient; (b) a stage II or III patient; or (c) a stage III or IV patient.

The term “patient-derived tumor sample” may include, for example, tumor material from surgical resection or biopsy (e.g. a cell from a biopsy of the patient). As used herein, the term “biopsy” includes a reference to tissue removed from the patient. The tissue may be removed using any suitable method, such as needle biopsy, aspiration, scraping, excision using surgical excision. Suitably the sample comprises total tumor material i.e. tumor infiltrating leukocytes (TIL), stroma and tumor cells The sample may optionally be a fragment of resected tumor. The sample may be obtained at one or more time points. Optionally, the sample can be subjected to one or more post-collection preparative or storage techniques (e.g. fixation, storage, freezing, lysis, homogenization, DNA or RNA extraction, cDNA conversion, ultrafiltration, dilution (e.g. with saline, buffer or a physiologically acceptable diluents etc.), concentration, evaporation, centrifugation, separation, filtration, etc.) prior to the material being analysed by the methods of the present invention. Optionally, steps (a) and (b) of the methods of the present invention may be preceded by the step of obtaining the patient-derived tumor sample from the patient.

In one embodiment of the invention, the three or more genes of Table 1 are at least 3, 4, 5, 6, 7, 8, 9, 10, 11, 12, 13, 14, and/or all the genes listed in Table 1 and/or any combination thereof. In a preferred method of the present invention, 14 or 15 genes are selected from Table 1.

In one embodiment of the invention, the three or more genes of Table 2A are at least 3, 4, 5, 6 and/or all of the genes listed in Table 2A and/or any combination thereof.

In one embodiment of the invention, the three or more genes of Table 2B are at least 3, 4, 5, 6 and/or all of the genes listed in Table 2B and/or any combination thereof.

In one embodiment of the invention, the three or more genes of Table 3 are at least 3, 4, 5, 6, 7, 8, 9, 10 and/or all of the genes listed in Table 3 and/or any combination thereof.

In one embodiment of the invention, the three or more genes of Table 4 are at least 3, 4, 5, 6, 7, 8, 9, 10, 11, 12, 13 and/or all of the genes listed in Table 4 and/or any combination thereof.

In one embodiment of the invention, the three or more genes of Table 14 are at least 3, 4, 5, 6, 7 and/or all of the genes listed in Table 14 and/or any combination thereof.

In one embodiment of the invention, the three or more genes of Table 15 are at least 3, 4, and/or all of the genes listed in Table 15 and/or any combination thereof.

In one embodiment of the invention, the three or more genes of Table 16 are at least 3, 4, and/or all of the genes listed in Table 16 and/or any combination thereof.

Accordingly, it will be understood that the invention may comprise determining the expression level of four or more, five or more, six or more etc. genes as listed in Table 1, 2A, 2B, 3, 4, 14, and/or 16 (see Tables below).

In at least some embodiments of the invention, the expression levels of fewer than 15, 14, 13, 12, 11, 10, 9, 8, 7, 6, 5 or 4 genes selected from the genes listed in Table 1, 2A, 2B, 3, 4, 14, and/or 16 are determined.

Preferably, the expression levels of 3 to 15, 3 to 14, 3 to 13, 4 to 15, 4 to 14, 4 to 13, 5 to 15, 5 to 14, 5 to 13, 6 to 15, 6 to 14, 6 to 13, 7 to 15, 7 to 14, 7 to 13, 8 to 15, 8 to 14, 8 to 13, 9 to 15, 9 to 14, 9 to 13, 10 to 15, 10 to 14, 10 to 13, 11 to 15, 11 to 14, 11 to 13, 12 to 15, 12 to 14 or 12 to 13 genes from the genes listed in Table 1, 2A, 2B, 3, 4, 14, 15 and/or 16 are determined.

Preferably, the three or more genes comprise (and optionally consist of):
(i) IL6 and TNF;
(ii) CCL2, CCL5 and CCR2;
(iii) CCL5, CCL2 and CXCL10;
(iv) IFNG, TNF and TLR3;
(v) CCL5, CCL2, CXCL10 and CCR2;
(vi) CCL2, CCL5, CCR2 and IL6;
(vii) CCL2, CCL5, CCR2, IL6 and NCR3;
(viii) CCL5, CCL2, CXCL10 and TLR3;
(ix) CCL5, CCL2, CXCL10, CCR2 and TLR3;
(x) CCL5, CCL2, CXCL10, IFNG, TNF and TLR3;
(xi) CCL5, CCL2, CXCL10, CCR2, IFNG, TNF and TLR3;
(xii) CXCL10, TLR3, TNF, IFNG and CCL5;
(xiii) CCL5, CCR2, CD8A, FCGR1A, IL6, NCR3, TLR3 and TLR;
(xiv) CCL2, CD8A, CXCL10, IL6, LTA, NCR3, TBX21 and TNF;
(xv) CCL2, CCL5, CCR2, CD8A, CXCL10, FCGR1A, IL6, NCR3, TBX21, TLR3, TLR4, IFNG and TNF;
(xvi) CCR2, CD8A, IL6, LTA and TLR3;
(xvii) CD8A, CXCL10, IL6, TLR3 and TLR4;
(xviii) CCL5, FCGR1A, IFNG, IL6, TLR3, TLR4 and TNF;
(xix) CCL5, CCR2, CD8A, FCGR1A, IFNG, IL6, and NCR3;
(xx) CCL2, CCL5, CCR2, CD8A, CXCL10, FCGR1A, IL6, NCR3, TBX21, TLR3 and TLR4;
(xxi) CCL2, CCR2, TLR3, TLR4, CCL5, IL6, NCR3, TBX21, CXCL10, IFNG, CD8A, FCGR1A, CEACAM8 and TNF;
(xxii) the genes common to Table 2 (Table 2A and/or Table 2B), Table 3 and Table 4;
(xxiii) the genes common to Table 2 (Table 2A and/or Table 2B), Table 3, Table 4, Table 14, Table 15 and Table 16;
(xxiv) any combination of the above gene sets.

In step (b) of the invention, the gene expression level information from the three or more genes listed in Table 1, 2A, 2B, 3, 4, 14, 15 and/or 16 may be used alone in classifying the patient, providing a prognosis, etc. or in combination with other information which may for example be genotypic, phenotypic or clinical information. Optionally, the gene expression level information from the three or more genes listed in Table 1, 2A, 2B, 3, 4, 14, 15 and/or 16 may be used with one or more of the following: expression level information from one or more additional genes which is/are not listed in Table 1, 2A, 2B, 3, 4, 14, 15 and/or 16 (herein referred to as “further marker genes”); staging information (stage I, II, III or IV), and classical histopathological measurements such as tumour nodularity and vascular invasion. Other factors which may be taken into account in step (b) of the invention include one or more of the following: gender, age, ethnicity, previous cancer history, hereditary factors (family history of cancer), weight, lifestyle factors such as diet, activity levels, alcohol consumption, recreational drug use, whether the patient is/was a smoker and extent of habit, disease etiology, viral infections like for example hepatitis viruses, liver function such as Model for End-Stage Liver Disease (MELD) system or Child-Pugh score (cirrhosis staging system) and exposure to ionizing radiation.

Various methods for using such additional information in combination with the gene expression level information from the three or more immune genes of the invention will be known to the persons skilled in the art. One such method would be to fit a multi-variate model (e.g. a cox regression model) which involves clinical parameters and signature as independent variables and death as a dependent variable. The model can then be used to divide the patients into “low” and “high” risk groups. In one embodiment, a multi-variate model which involves clinical parameters and signature as independent variables and death as a dependent variable is used to obtain a median hazard ratio and the median hazard ratio is used as a cut-off point. With regard to the utilisation of additional information in combination with the gene expression level information from the three or more immune genes of the invention, reference is made to Dusan Bogunovis et al. PNAS 2009, vol 106, no. 48, pp 20429-20434, the teachings of which are incorporated herein by reference. Also see FIG. 5 in this document.

Where step (b) utilises gene expression level information from one or more further marker genes, then step (a) may optionally comprise determining the expression level(s) of said one or more further marker genes, in addition to determining the expression levels of the three or more genes listed in Table 1, 2A, 2B, 3, 4, 14, 15 and/or 16.

In at least some embodiments of the invention, the expression level(s) of one or more further marker genes is/are not employed. Accordingly, in some embodiments of the invention the gene expression profile that is used in step (b) consists of expression level information of the three or more immune genes of the invention.

As used herein the term a “further marker gene” includes a reference to a gene whose level of expression is informative of, or of predictive value, in providing an HCC patient prognosis. As such, the expression level(s) of the one or more further marker genes may be usefully combined with the expression levels of the three or more immune genes of the invention when classifying or stratifying the patient, providing a patient prognosis (e.g. poor or good; long or short survival), monitoring disease progression, predicting efficacy of a therapeutic intervention, selecting treatment for the patient, or evaluating the efficacy of a therapeutic intervention.

Those skilled in the art will appreciate that the manner in which the one or more further marker genes may be employed in the methods of the present invention will depend on the marker gene. For example, it is envisaged that the expression of some further marker genes will be positively correlated with good patient outcomes. Conversely, it is envisaged that the expression of other further marker genes may be negatively correlated with good patient outcomes. Moreover, for some marker genes it may be necessary to quantify the expression of the gene (either in relation to polynucleotides derived therefrom (e.g. mRNA) or in relation to proteins/polypeptides encoded thereby) whilst for others it may merely be necessary to determine if expression of the marker gene is present or absent for the marker gene to be of predictive value.

Examples of further marker genes whose expression levels may usefully be employed in step (b) of the invention include immune-related genes and tumor-associated genes The following publications may also be useful in identifying possible further marker genes: Budhu et al. (2006) Cancer Cell 10:99-111; Lee et al. (2004) Hepatology 40:667-76; Hoshida et al. N Engl J Med 2008; 359:1995-2004.; Chen et al. Mol Biol Cell 2002; 13:1929-39; Lizuka et al. Lancet 2003; 361:923-9; Breuhahn et al. Cancer Res 2004; 64:6058-64; Ye et al. Nat Med 2003; 9:416-23; Midorikawa et al. Cancer Res 2004; 64:7263-70; Boyault et at Hepatology 2007; 45:42-52; Chiang et al. Cancer Res 2008; 68:6779-99 and Hoshida et al. Cancer Res 2009; 69:7385-92. These publications may also provide guidance on how such one or more further marker genes may be employed in analyzing HCC patients, such as to provide a prognosis.

The expression levels of the one or more further marker genes may be determined from the same patient-derived tumor sample as the three or more immune genes of the invention or from a different biological sample from the patient. Examples of sources of sample material for determining the expression of the one or more further marker genes include peripheral blood, tumor cells and non-tumor cells. The assay material may optionally be cells, tissue or serum.

The expression levels of the one or more further marker genes may be determined from the same patient-derived tumor sample as the three or more immune genes of the invention or from a different biological sample from the patient. Optionally, where the expression levels of one or more further marker genes are employed in the present invention, the expression levels of at least 4, 5, 6, 7, 8, 9, 10, 11, 12, 13, 14, 15, 16, 17, 18, 19, 20, 25, 30, 35, 40, 50, 60, 80, 100, 120, 150, 165, 180, 200, 225, 250, 275, 300, 325, 350, 375, 400, 425 or 450 further marker genes are employed.

Optionally, where the expression levels of one or more further marker genes are employed in the present invention, the expression levels of no more than 1, 2, 3, 4, 5, 6, 7, 8, 9, 10, 11, 12, 13, 14, 15, 16, 17, 18, 19, 20, 25, 30, 35, 40, 50, 60, 80, 100, 120, 150, 165, 180, 200, 225, 250, 275, 300, 325, 350, 375, 400, 425, 450, or 475 further marker genes are determined.

In at least some embodiments of the invention, in addition to determining the expression of the three or more genes listed in Table 1, 2A, 2B, 3, 4, 14, 15 and/or 16, and optionally also the expression of the one or more further marker genes, the expression level of one or more normalizing or control genes may be determined. This is discussed further below.

The expression levels of the three or more immune genes of the invention (and, if applicable, optionally also those of the one or more further marker genes) may be used to generate an expression profile. An expression profile of a particular sample is essentially a “fingerprint” of the state of the sample—while two states may have any particular gene similarly expressed, the evaluation of a number of genes simultaneously allows the generation of a gene expression profile that is characteristic of the state of the cell or tissue. Thus, an “expression profile” may be considered as referring to the collective pattern of gene expression by a particular cell type or tissue under given conditions at a given time. Where an “expression profile” is predictive it may be used synonymously with the term “gene signature”. The use of expression profiles allows normal tissue to be distinguished from, for example, cancerous tissue, or cancer tissue (e.g. biopsied material) to be compared with tissue from surviving cancer patients (e.g. patients known to have a good or poor disease outcome). Comparing expression profiles in different cancer states identifies genes (e.g. up- and down-regulated genes) that are important in each of these states. Molecular profiling may distinguish subtypes of a currently collective disease designation, e.g., different forms or stages of a cancer. In the present invention, the expression profile may be used in classifying or stratifying the patient, providing a patient prognosis (e.g. poor or good; long or short survival), monitoring disease progression, predicting efficacy of a therapeutic intervention, selecting treatment for the patient, or evaluating the efficacy of a therapeutic intervention etc., i.e. the expression profile may be used in step (b) of the methods of the present invention. As will be appreciated from the above discussion, the expression profile may be used alone in step (b) or with other information such as staging information, cancer history etc.

In the present invention, the expression levels of the three or more immune genes of the invention (and optionally those of any further marker genes) are used to analyze HCC patients, e.g. to provide a prognosis for the patient. Preferably, the expression levels of the three or more immune genes of the invention (and, if applicable, optionally also those of the one or more further marker genes) are normalized. Normalization enables factors which may cause results to vary between assays to be minimized or corrected for (normalized away). Potential sources of variation will obviously depend on how the expression levels are determined but may, for example, include: variations in the amount or quality of RNA or other assayed material, variations in hybridization conditions, label intensity, or “reading” efficiency. In a preferred embodiment, the expression levels of the immune genes of the invention (and optionally those of any further marker genes) are divided by the expression level of a normalizing gene to thereby normalize the measurements. Optionally, the normalizing gene is a constitutively expressed house-keeping gene such as ACTB, the beta-actin gene, the transferrin receptor gene, the GAPDH gene or Cyp1. Other examples of normalizing genes includes RPS13, RPL27, RPS20 and OAZ1. Reference is also made to Evidence Based Selection of Housekeeping Genes by Hendrik et al. Plos one 2007 for further examples of housekeeping genes which may be employed. Software may be used to normalize the expression levels. MxPro software (Stratagene) may optionally be used. In a particularly preferred embodiment of the invention, the expression levels of the immune genes of the invention (and, if applicable, optionally also those of the one or more further marker genes) are normalized to ACTB using MxPro software (Stratagene).

Alternatively or additionally, normalization can be based on the mean or median value of each or all of the assayed genes or a large subset thereof (global normalization approach). In a preferred embodiment, alternative or additional normalization of the immune genes of the invention is performed with the median value of each particular gene according to training cohort (Sg cohort) (See Table 10 in Example 2 for the median values of each gene from Sg as the training cohort).

For the avoidance of doubt, the terms “gene expression level information”, “expression levels”, and “expression values” and like expressions include (unless the context indicates otherwise) a reference to the expression levels themselves (i.e. absolute expression levels) or data derived therefrom e.g. where the expression level values have been transformed, for example to provide normalized expression values, or relative expression values. Relative expression values may suitably be obtained by normalizing the expression levels to a housekeeping gene and then to median values of the particular gene from individual cohorts of patients such as training or validation cohorts (see e.g. Table 10). The term “gene expression level information” may refer to the gene expression level information of the three or more immune genes of the invention and/or, if applicable, the gene expression level information of the one or more further marker genes, unless the context indicates otherwise. As discussed below, gene expression level information may be generated by quantifying expression of a peptide or polypeptide encoded by the gene, or a polynucleotide derived from the gene (e.g. RNA transcribed from the gene, any cDNA or cRNA produced therefrom, or any other nucleic acid derived therefrom).

Persons skilled in the art will be able to appreciate that the gene expression level information may be used in various ways to analyze the patient e.g. to stratify or classify the patient, or provide a prognosis etc. As mentioned above, the patient may be analyzed on the basis of the expression levels alone, or on the basis of a combination of the gene expression level information with other information such as clinical information.

In at least some embodiments of the methods of the present invention, step (b) comprises deriving a value from the expression levels of a combination of the three or more immune genes of the invention (and, if applicable, optionally also those of the one or more further marker genes) and comparing the value with a threshold value. A determination that the value derived from the gene combination is below or a above a threshold value (e.g. as defined by an algorithm such as the SVM algorithm) indicates a particular prognosis (e.g. a good or poor prognosis). Preferably, in at least some embodiments a determination that the value derived from the gene combination is below a threshold value indicates a poor prognosis whilst a determination that the value derived from the gene combination is above a threshold value indicates a good prognosis. Conversely, in other embodiments of the invention a determination that the value derived from the gene combination is below a threshold value indicates a good prognosis whilst a determination that the value derived from the gene combination is above a threshold value indicates a poor prognosis.

In the SVM (Support Vector Machine) algorithm described below (“Algorithm 1”) the threshold value is 0 and the determination that the value derived from the gene combination is below a threshold value as defined by the algorithm indicates a poor prognosis whilst above the threshold value indicates a good prognosis. The hyperplane as determined from machine-learning process using training cohort is a general plane that separates the space into two half spaces. It divides the 2 classes of above and below the threshold value of zero. Details of how to derive the value from the gene combination may be found in Algorithm 1 below. The formula given in the algorithm is used to derive a value from the levels of any combination of genes and the resulting value is compared to the threshold.

Support Vector Machines are based on the concept of decision planes that define decision boundaries. A decision plane is one that separates between a set of objects having different class memberships. With regard to use of threshold values and the use of Support Vector Machines reference is made to Burges. A tutorial on support vector machines for pattern recognition. Data mining and Knowledge discovery, 2, 121-167 (1998), the teachings of which are incorporated herein by reference.

In at least some embodiments of the invention, the expression levels of the three or more immune genes of the invention (and, if applicable, optionally also those of the one or more further marker genes) take the form an expression profile. The expression levels may for example be normalized expression levels or relative expression levels etc. Methods of the invention are provided where step (b) comprises determining the similarity of the expression profile to one or more templates of a particular HCC type or prognosis (e.g. good or poor prognosis, long or short survival), wherein the degree of similarity (including dissimilarity) of the expression to a template (or templates) of a particular HCC type or prognosis indicates whether the patient has the particular HCC type or prognosis respectively. Suitably, similarity is indicative of a particular HCC type or prognosis, whereas dissimilarity is indicative that the patient does not have the particular HCC type or prognosis. As discussed herein, other information (e.g. staging information) may also be used in analyzing the patient, e.g. in providing a particular prognosis or classifying/stratifying the patient into a particular subtype.

A template of a particular prognosis suitably comprises gene expression levels characteristic (i.e. representative) of the particular HCC type or prognosis. In some embodiments of the invention, the template may be determined as described in steps 1 to 2 or 1 to 3 of algorithm 3 (optionally with different values being assigned to the “bad” prognosis-correlated genes and “good” prognosis-correlated genes, such as a positive or negative multiples of the values used in Step 2 (1 and −1)). In some embodiments of the invention, each expression level in the template is an average (mean, mode or median) of expression levels of the gene in a plurality of individuals (e.g. at least 2, 3, 4, 5, 8, 10, 12, 15, 20, 30, 40, 50, 60, individuals) determined as having said particular HCC type or prognosis/outcome.

A poor prognosis template accordingly comprises gene expression values characteristic of poor prognosis patients, whilst a good prognosis template accordingly comprises gene expression values characteristic of good prognosis patients. In a preferred embodiment, each of the gene expression values in the poor or good prognosis template is an average (mean, mode or median) of expression levels of the gene in a plurality of poor or good outcome patients, respectively.

In one embodiment, step (b) comprises determining the similarity of the expression profile to a good prognosis template and/or a poor prognosis template, and wherein said patient is classified as having: (i) a good prognosis if said expression profile is similar to the good prognosis template and/or is dissimilar to the poor prognosis template; or (ii) a poor prognosis if said expression profile is dissimilar to the good prognosis template and/or is similar to the poor prognosis template. In one embodiment, the similarity between the expression profile and the template is determined as being “similar” or “dissimilar” where the similarity is above or below a predetermined threshold respectively. In another embodiment, the similarity between the expression profile and the template is determined as being of “similar” or “dissimilar” where the similarity is below or above a predetermined threshold respectively.

In one embodiment, step (b) comprises determining the similarity of the expression profile to a good prognosis template and a poor prognosis template, and wherein said patient is classified as having: (i) a good prognosis if said expression profile has a higher similarity to said good prognosis template than to said poor prognosis template; or (ii) a poor prognosis if said expression profile has a higher similarity to said poor prognosis template than to said good prognosis template.

In at least some embodiments of the invention, similarity between a patient's expression profile and a template is represented by a distance between the patient's expression profile and the template. In one embodiment, a distance below a given value indicates similarity, whereas a distance equal to or greater than the given value indicates dissimilarity. In one embodiment, distance is “cosine distance”. Methods of calculating cosine distances will be known to those skilled in the art but cosine distance may optionally be calculated using the formula in step 4 of Algorithm 3. With regard to the use of cosine distance, reference is also made to P.-N. Tan, M. Steinbach & V. Kumar, “Introduction to Data Mining”, Addison-Wesley (2005), ISBN 0-321-32136-7, chapter 8; page 500, the teaching of which is incorporated herein by reference. Other methods of calculating distance will be known to those skilled in the art and include, for example, Euclidean distance and Hamming distance. With regard to Euclidean distance reference is made to Elena Deza & Michel Marie Deza (2009) Encyclopedia of Distances, page 94, Springer, the teaching of which is incorporated herein by reference. With regard to Hamming distance, reference is made to Hamming, Richard W. (1950), “Error detecting and error correcting codes”, Bell System Technical Journal 29 (2): 147-160, MR0035935, the teaching of which is incorporated herein by reference.

Patients may be analyzed (e.g. classified, provided with a prognosis, treatment selected etc.) using the gene expression information using any means known in the art. In general, the expression values of a training cohort are used to build a mathematical model which takes gene expression values as input and output the prognosis outcome. The mathematical model is then used to classify (e.g. assign a poor or good prognosis to) new patients.

There are many machine learning algorithms which may be used in the present invention e.g. decision trees, artificial neural networks, genetic algorithms, Bayesian networks, etc. and accordingly in at least some embodiments of the invention step (b) of the methods of the present invention is performed using a machine learning algorithm.

In preferred embodiments of the invention step (b) may be performed using software specifically designed or adapted to perform step (b).

Preferably, step (b) of the methods of the present invention is performed using at least one algorithm. Preferably, the “at least one algorithm” is 1, 2, 3, 4 or 5 algorithms. Preferably, enhanced accuracy, specificity and/or sensitivity is achieved with the combination of 2 or more algorithms.

Preferably, step (b) is performed using a SVM algorithm, a KNN algorithm or a combination of an SVM and a KNN algorithm. Enhanced accuracy, specificity and sensitivity can be achieved with the combination of the SVM and KNN algorithms. As discussed above, information (e.g. staging information) may optionally be combined.

Where step (b) is performed using the SVM algorithm, a preferred embodiment provides that: the three or more genes of Table 2A are at least 3, 4, 5, 6 and/or all of the genes listed in Table 2A and/or any combination thereof; the three or more genes of Table 2B are at least 3, 4, 5, 6 and/or all of the genes listed in Table 2B and/or any combination thereof; the three or more genes of Table 1 are at least 3, 4, 5, 6, 7, 8, 9, 10, 11, 12, 13, 14 and/or all of the genes listed in Table 1 and/or any combination thereof; the three or more genes of Table 14 are at least 3, 4, 5, 6, 7 and/or all of the genes listed in Table 14 and/or any combination thereof; the three or more genes of Table 15 are at least 3, 4 and/or all of the genes listed in Table 15 and/or any combination thereof; or the three or more genes of Table 16 are at least 3, 4 and/or all of the genes listed in Table 16 and/or any combination thereof.

In a preferred embodiment of the invention, step (b) is performed by application of an SVM algorithm as described in Algorithm 1 and classifies a patient as having a good or poor prognosis.

Where step (b) is performed using the KNN algorithm, a preferred embodiment provides that: the three or more genes of Table 3 are at least 3, 4, 5, 6, 7, 8, 9, 10 and/or all of the genes listed in Table 3 and/or any combination thereof; or the three or more genes of Table 1 are at least 3, 4, 5, 6, 7, 8, 9, 10, 11, 12, 13, 14 and/or all of the genes listed in Table 1 and/or any combination thereof.

In a preferred embodiment of the invention, step (b) is performed by application of a KNN algorithm as described in Algorithm 2 and classifies a patient as having a good or poor prognosis.

Where step (b) is performed using the combination of an SVM and a KNN algorithm, a preferred embodiment provides that: the three or more genes are at least 3, 4, 5, 6, 7, 8, 9, 10, 11, 12 or 13 of the genes selected from the group consisting of: CCL2, CCL5, CCR2, CD8A, CXCL10, FCGR1A, IL6, NCR3, TBX21, TLR3, TLR4, IFNG and TNFA.

In some embodiments of the invention, step (b) is performed using an NTP algorithm. Preferably, when step (b) is performed using an NTP algorithm, the patient is a patient with stage II or III HCC. The NTP 14-immune genes prediction method is able to predict survival of HCC patients from Stage II & III which usually have very similar survival profiles (p=ns). This is very useful for HCC patients from Stage II or III where tumor staging alone is not able to segregate patients into good or poor prognosis.

Where step (b) is performed using an NTP algorithm, a preferred embodiment provides that: the three or more genes of Table 4 are at least 3, 4, 5, 6, 7, 8, 9, 10, 11, 12, 13 and/or all of the genes listed in Table 4 and/or any combination thereof, or the three or more genes of Table 1 are at least 3, 4, 5, 6, 7, 8, 9, 10, 11, 12, 13, 14 and/or all of the genes listed in Table 1 and/or any combination thereof.

To determine the expression level of a gene any suitable method in the art may be used. Gene expression may be assessed in relation to protein/polypeptide encoded by the gene, such as by immunohistochemistry, Western blotting, mass-spectrometry, flow cytometry, luminex, ELISA, RIA, etc. Alternatively, the expression level may be determined in relation to a polynucleotide derived from the gene, e.g. mRNA or nucleic acids derived therefrom such as cDNA or amplified DNA. Nucleic acid may optionally be amplified prior to or during its quantification. Examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

To determine the expression levels of the immune genes of the invention (and, if applicable, optionally also those of the one or more further marker genes) RT-PCR, qRT-PCR, qPCR, hybridization or sequencing analysis may optionally be used.

In a preferred embodiment of the present invention a microarray kit or quantitative PCR (qPCR) is used. Accordingly, in at least some embodiments of the methods of the present invention, the method comprises use of a microarray kit or qPCR to determine the expression level of any or all of the genes listed in Table 1 (and, if applicable, optionally also those of the one or more further marker genes). Preferably, prior to carrying out qPCR RNA is extracted from the patient-derived tumor sample and/or the RNA is reverse transcribed. Methods for generating cDNA from mRNA are well known in the art. Typically, purified mRNA is primed using a polydT sequence or random primers. A reverse transcriptase is then employed to synthesise DNA complementary to the mRNA sequence. Second strand synthesis is then performed.

The present invention provides microarrays for use in the methods of the invention, which microarrays comprise a plurality of probes capable of hybridizing to the said three or more genes selected from the genes listed in Table 1, the genes listed in Table 2A or Table 2B, the genes listed in Table 3, the genes listed in Table 4, the genes listed in Table 14, the genes listed in Table 15, and/or the genes listed in Table 16. Preferably, there is provided a microarray in which at least 50%, 60%, 70%, 80%, 90% or 95% of the probes are probes which are capable of hybridizing to the said three or more genes selected from the genes listed in Table 1, the genes listed in Table 2A or Table 2B, the genes listed in Table 3 Table 3, the genes listed in Table 4, the genes listed in Table 14, the genes listed in Table 15, and/or the genes listed in Table 16. Optionally, the microarray may be provided in a container or with instructions for use in a method of the present invention so as to thereby provide a microarray kit.

Step (b) of the methods of the present invention can be performed by using a computer. Thus, in a preferred embodiment step (b) is performed using a computer system or computer software product of the invention. Computer software products of the invention typically include computer readable media having computer-executable instructions for performing step (b) of the methods of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. The software may optionally include instructions for the computer system's processor to receive data structures that include the level of expression of the three or more immune genes of the invention (and optionally one or more further markers) and optionally further information to be used in the analysis e.g. staging information, patient's age, weight etc. The software may include mathematical routines for analyzing the data.

The present invention also includes a computer system programmed to perform step (b) of the methods of the present invention. A computer system comprises internal components linked to external components. The internal components of a typical computer system include a processor element interconnected with a main memory. The external components may include mass storage. Other external components include a user interface device (e.g. monitor) together with an inputting device (e.g. a “mouse” and/or keyboard). Typically, a computer system is also linked to a network, such as the Internet. This network link allows the computer system to share data and processing tasks with other computer systems.

The invention will now be further defined in terms of “aspects” of the invention. It is intended that where appropriate the above overview of the invention can be used to provide guidance on the interpretation and implementation of the aspects of the invention set out below.

A first aspect of the invention provides a method of analysing a patient with HCC, wherein the method comprises:
(a) determining the expression levels of three or more genes in a patient-derived tumor sample wherein the said three or more genes are selected from the genes listed in Table 1; the genes listed in Table 2A or Table 2B; the genes listed in Table 3, the genes listed in Table 4, the genes listed in Table 14, the genes listed in Table 15, and/or the genes listed in Table 16; and
(b) using the expression levels determined in step (a) in one or more of the following: stratifying or classifying the patient, providing a prognosis, monitoring disease progression, predicting efficacy of a therapeutic intervention, selecting treatment for the tumor, or evaluating the efficacy of a therapeutic intervention.

Optionally, the expression level information obtained in step (a) of the first aspect of the invention may be used in conjunction with other information (e.g. staging information, expression level information from one or more further marker genes) when stratifying or classifying the patient, providing a prognosis, predicting the efficacy of a therapeutic intervention, selecting treatment for the tumor, or evaluating the efficacy of a therapeutic intervention.

Optionally, step (a) of the first aspect of the invention further comprises determining the expression level(s) of one or more further marker genes.

Preferably, the expression levels are normalized expression levels.

In accordance with the methods of the present invention, a patient may be classified or provided with a prognosis (e.g. a poor or good prognosis, or short or long survival etc.). Such prognostic information may optionally be used to stratify or classify the patient, monitor disease progression, predict efficacy of a therapeutic intervention, select treatment for the tumour, or evaluate the efficacy of a therapeutic intervention.

By “three or more genes” we include 4, 5, 6, 7, 8, 9, 10, 11, 12, 13, 14 or 15 genes.

In one embodiment of the first aspect of the invention there is provided a method of providing a an HCC human patient with a good or a poor prognosis, wherein the method comprises: (a) determining the expression levels of three or more (and preferably five or more) genes in a tumor sample derived from said patient, which tumor sample comprises total tumor material, wherein the said three or more genes are selected from at least one list of genes selected from the group consisting of the genes listed in Table 1; the genes listed in Table 2A; the genes listed in Table 2B; the genes listed in Table 3; the genes listed in Table 4, the genes listed in Table 14, the genes listed in Table 15, and the genes listed in Table 16, and wherein the expression levels may optionally be relative expression levels and/or normalized expression levels; and (b) using the expression levels determined in step (a) to provide the patient with said prognosis, optionally by determining the similarity of an expression profile comprising the expression levels determined in step (a) to a good prognosis template which comprises gene expression levels characteristic of good prognosis patients and a poor prognosis template which comprises gene expression levels characteristic of poor prognosis patient, wherein a higher similarity of said expression profile to said good prognosis template indicates a poor prognosis and a higher similarity to said poor prognosis template than to said good prognosis template indicates a poor prognosis. Preferably, each of the gene expression values in the poor or good prognosis template is an average (mean, mode or median) of expression levels of the gene in a plurality of poor or good outcome patients, respectively.

A second aspect of the invention provides a method of classifying a patient with HCC as having a poor or good prognosis comprising the steps of:
(a) determining the expression levels of three or more genes in a patient-derived tumor sample, wherein the gene(s) are selected from the genes listed in Table 1; the genes listed in Table 2A or Table 2B; the genes listed in Table 3; the genes listed in Table 4; the genes listed in Table 14; the genes listed in Table 15; and/or the genes listed in Table 16; and
(b) using the expression levels determined in step (a) in classifying the patient as having a poor or good prognosis.

A third aspect of the invention provides a method of classifying a patient with HCC as having a poor or good prognosis comprising the steps of:
(a) determining the expression levels of three or more genes in a patient-derived tumor sample, wherein the gene(s) are selected from the genes listed in Table 1; the genes listed in Table 2A or Table 2B; the genes listed in Table 3; the genes listed in Table 4; the genes listed in Table 14; the genes listed in Table 15; and/or the genes listed in Table 16; and
(b) classifying the patient as having a short or long survival based on the expression levels determined in step (a), in which the patient has HCC.

In at least some embodiments of the invention, the terms “long survival” and “short survival” are used synonymously with good and poor prognosis respectively. In at least some embodiments, the term “short survival” refers to less than 3, 4, 5 or 6 years survival. In at least some embodiments, the term “long survival” refers to more than or equal to 3, 4, 5 or 6 years survival.

In at least some embodiments of the invention, the term “long survival” refers to when the gene expression profile has a higher similarity to a long survival template than a short survival template.

In at least some embodiments of the invention, the term “long survival” refers to when the gene expression profile is similar to a long survival template and/or is dissimilar to a short survival template.

In at least some embodiments of the invention, the term “short survival” refers to when the gene expression profile has a higher similarity to a short survival template than a long survival template.

In at least some embodiments of the invention, the term “long survival” refers to when the gene expression profile is similar to a short survival template and/or is dissimilar to a long survival template.

A fourth aspect of the invention provides a method of classifying a patient with HCC as having a poor or good prognosis comprising the steps of:
(a) determining the expression levels of five or more genes in a patient-derived tumor sample, wherein the gene(s) are selected from the genes listed in Table 1; the genes listed in Table 2A or Table 2B; the genes listed in Table 3; the genes listed in Table 4, the genes listed in Table 14, the genes listed in Table 15, and/or the genes listed in Table 16; and
(b) classifying the patient as having a short or long survival based on the expression levels determined in step (a), in which the patient has HCC.

A fifth aspect of the invention provides a method for evaluating the efficacy of a therapeutic intervention for treating HCC patients comprising the steps of:
(a) determining the expression levels of three or more genes selected from the genes listed in Table 1; the genes listed in Table 2A or Table 2B; the genes listed in Table 3; the genes listed in Table 4; the genes listed in Table 14; the genes listed in Table 15; and/or the genes listed in Table 16; and
(b) using the expression levels determined in step (a) in evaluating the efficacy of a therapeutic intervention.

As will be appreciated from the above discussion, optionally, the expression level information obtained in step (a) of the first aspect of the invention may optionally be used in conjunction with other information (e.g. staging information) when stratifying or classifying the patient, providing a prognosis, predicting the efficacy of a therapeutic intervention, selecting treatment for the tumor, or evaluating the efficacy of a therapeutic intervention.

Step (a) may be performed at one or more time points (preferably at least at 1, 2, 3, 4 or 5 time points) such as before, during and/or after the therapeutic intervention. In this way, the effect of the therapeutic intervention on the gene expression levels may be determined and this information used in step (b) so as to enable an evaluation of the efficacy of the therapeutic intervention. Optionally, step (a) is performed only at one time point, such as after treatment. Where step (a) is performed after treatment, the expression level information may be compared with that of a non-treated control group. In a preferred embodiment of the method of the fifth aspect of the invention, the expression levels determined in step (a) are compared with those of a non-treated control and this comparison is used to evaluate the efficacy of the therapeutic intervention (optionally in combination with other information, such as clinical information etc.).

In at least some embodiments of the fifth aspect of the invention, step (a) is performed before and after the therapeutic intervention, and optionally also during the therapeutic intervention.

A sixth aspect of the invention provides a method of evaluating the efficacy of a therapeutic intervention for treating HCC patients comprising the steps of:
(a) determining the expression levels of three or more genes in a patient-derived tumor sample, wherein the gene(s) are selected from the genes listed in Table 1; the genes listed in Table 2; the genes listed in Table 3; the genes listed in Table 4; the genes listed in Table 14; the genes listed in Table 15; and/or the genes listed in Table 16; and
(b) using the expression levels determined in step (a) in classifying the patient as having a poor or good prognosis, and in which classification of a patient by step (b) is monitored before, during and/or after the therapeutic intervention.

A seventh aspect of the invention provides a method for evaluating the efficacy of a therapeutic intervention for treating HCC patients comprising the steps of:
(a) determining the expression levels of three or more genes in a patient-derived tumor sample, wherein the gene(s) are selected from the genes listed in Table 1; the genes listed in Table 2; the genes listed in Table 3; the genes listed in Table 4; the genes listed in Table 14; the genes listed in Table 15; and/or the genes listed in Table 16; and
(b) classifying the patient as having a short or long survival based on the expression levels determined in step (a), in which the patient has HCC, and in which classification of a patient by step (b) is monitored before, during and/or after the therapeutic intervention.

In the sixth and seventh aspects of the invention, the classification of a patient by step (b) is monitored at one or more time points (preferably at least at 1, 2, 3, 4 or 5 time points). The classification of a patient by step (b) is preferably monitored before and after the therapeutic intervention; during the therapeutic intervention; before and during the therapeutic intervention; or during and after the therapeutic intervention. In one embodiment the classification of a patient by step (b) is monitored before, during and after the therapeutic intervention.

The immune signature of the present invention may be useful in identifying or selecting agents effective in treating HCC. In such instances, the expression levels of the three or more immune genes of the invention may serve as a surrogate biomarker for drug selection or drug efficacy. In a preferred embodiment of the fifth, sixth and seventh aspects of the invention the “therapeutic intervention” is experimental treatment. The patient may be receiving treatment with one or more agents which is/are undergoing experimental or clinical trials.

In one embodiment of the fifth, sixth and seventh aspects of the invention, step (a) is performed at multiple time points (e.g. before and during treatment; before and after treatment; periodically during treatment, or before, during and after treatment). In this way the expression profile of the patient can be assessed as the treatment progresses and the efficacy (if any) of the therapeutic intervention (e.g. candidate drug) can be determined.

In a preferred embodiment of the fifth, sixth and seventh aspects of the invention the therapeutic intervention is a neoadjuvant treatment.

In the methods of the present invention, gene expression level information may be used in selecting treatment for the patient. Accordingly, in at least some embodiments of the invention, the patient is stratified or classified for particular treatment, or the prognosis is used in selecting treatment for the patient. Optionally, a method of the present invention may comprise the further step of identifying a patient as having a particular prognosis (e.g. a poor or good prognosis, long or short survival), and selecting the patient for therapy or follow-up. For example, in one embodiment a patient having a good prognosis or long survival is selected for immunotherapy and/or liver transplantation.

An eighth aspect of the invention provides a method of treating a patient characterised as a patient having either good or poor prognosis according to the method of any one of the first to seventh aspects of the invention, wherein said patient is administered with a hepatocellular-carcinoma immunotherapy or any other alternative treatments.

A ninth aspect of the invention provides the use of immunotherapy or any other alternative treatment for hepatocellular-carcinoma in the preparation of a medicament for the treatment of patients characterized as having either good or poor prognosis according to the method of any one of the first to seventh aspects of the invention.

By “alternative treatment” it is included, for example, surgical intervention, liver transplantation, chemotherapy with a given drug or drug combination, radiation therapy, cell therapy, antibody therapy, gene therapy, and neoadjuvant treatment.

A tenth aspect of the invention provides a kit for use in any one of the first to seventh aspects of the invention, wherein the kit comprises reagents for determining the expression of said three or more genes (or five or more genes in the case of the fourth aspect of the invention) selected from the genes listed in Table 1, the genes listed in Table 2A or Table 2B, the genes listed in Table 3, the genes listed in Table 4, the genes listed in Table 14, the genes listed in Table 15, and/or the genes listed in Table 16 and wherein the kit further optionally comprises instructions for use. The kit may be promoted, distributed, or sold as a unit for performing the methods of the present invention.

Preferably, the kit comprises a set of probes and/or primers which comprise a plurality of oligonucleotides capable of hybridising to the said three or more genes selected from the genes listed in Table 1, the genes listed in Table 2A or Table 2B, the genes listed in Table 3, the genes listed in Table 4, the genes listed in Table 14, the genes listed in Table 15, and/or the genes listed in Table 16.

Preferably, the kit comprises primers for amplification of said three or more genes selected from the genes listed in Table 1, the genes listed in Table 2A or Table 2B, the genes listed in Table 3, the genes listed in Table 4, the genes listed in Table 14, the genes listed in Table 15, and/or the genes listed in Table 16.

In one embodiment, the kit may comprise a microarray (see above discussion in relation to microarrays) to thereby provide a microarray kit.

The kits of the tenth aspect of the invention may include any and all components necessary to perform a method of the invention.

In one embodiment of the tenth aspect of the invention, the kit comprises software wherein step (b) of the methods of the invention may be performed using the software.

As discussed above, the methods of the present invention may optionally employ one or more of the following algorithms.

### Algorithm 1

SVM (Support Vector Machine) decision function of an input vector x for a patient sample is

D(X)=W·X+b,

$$where {in}_{k} W = {\sum }^{ } {α}_{k} {y}_{k} {X}_{k} , \\ and$$

$$b = < {y}_{k} - W . {X}_{k} > ,$$

the weight vector W is a linear combination of training patterns Xₖ,
yₖ encodes the class binary value +1 or −1,
αₖ is an estimated parameter,
X represents the expression level of genes of Table 1.
If D(X)>0=>X is in class (+);
if D(X)<0=>X is in class (−); or
if D(X)=0, decision boundary.

A determination that the gene combination(s) are below a threshold value as defined by the SVM algorithms, indicates poor prognosis. A determination that the gene combination(s) are above a threshold value as defined by the SVM algorithms, indicates good prognosis.

### Algorithm 2

### KNN (K-Nearest Neighbour)

KNearest Neighbour algorithm makes classifications for test set from training set. For each patient sample of the test set, the k nearest (in Euclidean distance) patient samples in training set are found, and the classification is decided by majority vote, with ties broken at random. If there are ties for the kth nearest neighbors, all candidates are included in the vote.

The Euclidean distance between two patients is given by:

$$d ( {x}_{i} , {x}_{j} ) = \sqrt{{\sum }_{k = 1}^{n} {( {x}_{ik} - {x}_{jk} )}^{2}}$$

Wherein xᵢ=(xᵢ₁, xᵢ₂, . . . , xᵢₖ, xᵢₙ) is gene expression level for patient sample i; xⱼ=(xⱼ₁, xⱼ₂, . . . , xⱼₖ, . . . , xⱼₙ) is gene expression level for patient sample j; n is the total number of genes; xᵢₖ and xⱼₖ are expression level of gene k of sample i and j respectively.

KNN needs the level of expression from the training cohort in order to run the predictive algorithm. KNN selects the K number of closest “neighbor” patients, whose gene expression profiles are most similar to that of the patient of interest. The outcomes of the K neighbors are known. If majority of them has poor prognosis, KNN will give a poor prognosis prediction. Accordingly, a determination that the gene expression profile is similar to a good prognosis template as defined by the KNN algorithms, indicates a good prognosis; a determination that the gene expression profile is dissimilar to a good prognosis template as defined by the KNN algorithms, indicates a poor prognosis.

### Algorithm 3

### NTP (Nearest Template Prediction)

### Step 1:

NTP selects genes positively or negatively correlated with survival using the Cox score given by the following formula.

$$cox = [ {\sum }_{k = 1}^{K} ( {x}_{k}^{*} - {d}_{k} {\overline{x}}_{k} ) ] / {[ {\sum }_{k = 1}^{K} ( {d}_{k} / {m}_{k} ) {\sum }_{i ∈ {R}_{k}}^{ } {( {x}_{i} - {\overline{x}}_{k} )}^{2} ]}^{1 \text{/} 2}$$

Where i is indices of samples, xᵢ is gene expression level for sample i, tᵢ is time for sample i, kε1, . . . , K is indices of unique death times z₁, z₂, . . . , zₖ, dₖ is number of death at time zₖ, mₖ is number of samples in Rₖ=i:tᵢ≧zₖ, x*ₖ=Σₜ_{ᵢ}_{=z}_{ₖ} xᵢ, and x̅ₖ=Σ_{iεR}_{ₖ} xᵢ/mₖ.

Gene correlated with poor prognosis has positive cox score.

### Step 2:

A hypothetical sample serving as the template of “poor” prognosis was defined as a vector having the same length as the predictive signature. In this template, a value of 1 was assigned to “poor” prognosis-correlated genes and a value of −1 was assigned to “good” prognosis-correlated genes. And then each gene was weighted by the absolute value of the corresponding Cox score.

### Step 3:

The template of “good” prognosis was similarly defined.

### Step 4:

For each sample, a prediction was made based on the proximity measured by the cosine distance to either of the two templates. A sample closer to the template of “poor” prognosis was predicted as having poor prognosis.

The cosine distance between two patients is given by:

$$d ( {x}_{i} , {x}_{j} ) = 1 - \frac{{\sum }_{k = 1}^{n} {x}_{ik} {x}_{jk}}{\sqrt{{\sum }_{k = 1}^{n} {x}_{ik}^{2}} \sqrt{{\sum }_{k = 1}^{n} {x}_{jk}^{2}}}$$

Wherein xᵢ=xᵢ₁, xᵢ₂, . . . , xᵢₖ, . . . , xᵢₙ) is gene expression level for patient sample i; xⱼ=(xⱼ₁, xⱼ₂, . . . , xⱼₖ, . . . , xⱼₙ) is gene expression level for patient sample j; n is the total number of genes; xᵢₖ and xⱼₖ are expression level of gene k of sample i and j respectively.

NTP is a simple, yet flexible, nearest neighbour-based method designed to capture information from a certain pattern (e.g. gene expression patterns) as related to poor or good prognosis. Cox score is calculated for each gene depending on whether it's ON (+1) or OFF (−1) in the relevant biological functions/outcomes (e.g. poor vs good prognosis). The advantage of this method is that it is less sensitive to differences in experimental and analytical conditions, applicable to each single patient and it avoids the problem of setting an arbitrary cut-off of survival time.

NTP calculates the dissimilarity (or distance) of a patient's gene expression to a good/poor prognosis template. If the distance to poor prognosis template is smaller than the distance to good prognosis template, the patient is predicted to have poor prognosis. Accordingly, determination that the gene expression profile is dissimilar to a good prognosis template as defined by the NTP algorithms, indicates a poor prognosis; a determination that the gene combination(s) are dissimilar to a poor prognosis template as defined by the NTP algorithms, indicates a good prognosis.

### Computer System and Computer Program

It will be apparent to the person skilled in the art that the methods and algorithms described herein may be implemented as one or more computer programs executable within a computer system.

For example, FIG. 13 depicts a schematic flowchart illustrating the exemplary method 100 of analysing a patient with HCC described hereinbefore according to embodiment(s) of the present invention. The method comprises a step 102 of (a) determining the expression levels of three or more genes in a patient-derived tumor sample wherein the said three or more genes are selected from the genes listed in Table 1; the genes listed in Table 2A or Table 2B; the genes listed in Table 3; the genes listed in Table 4, the genes listed in Table 14, the genes listed in Table 15, and/or the genes listed in Table 16; and a step 104 of (b) using the expression levels determined in step (a) in one or more of the following: stratifying or classifying the patient, providing a prognosis, monitoring disease progression, predicting efficacy of a therapeutic intervention, selecting treatment for the tumor, or evaluating the efficacy of a therapeutic intervention.

The computer program 100 comprises a set of executable instructions, which when executed by the computer system, causes the computer system to perform one or more of the methods, method steps or algorithms described herein.

For example, FIG. 14 depicts an exemplary computer system 200 for executing the computer program according to an embodiment of the present invention.

The computer system 200 may comprise a computer module 202, input modules such as a keyboard 204 and a mouse 206, and a plurality of output or peripheral devices such as a display 208 and a printer 210.

The computer module 202 may be connected to a computer or communication network 212 via a suitable transceiver device 214, to enable access to e.g. the Internet or other network systems such as Local Area Network (LAN) or Wide Area Network (WAN).

The computer module 202 in the example may comprise a processor unit 218 and a memory unit. For example, the memory unit may comprise a Random Access Memory (RAM) 220 and a Read Only Memory (ROM) 222. The computer module 202 may further comprise a number of Input/Output (I/O) interfaces, for example I/O interface 224 to the display 208, and I/O interface 226 to the keyboard 204.

The components of the computer module 202 typically communicate via an interconnected bus 228 and in a manner known to the person skilled in the relevant art.

The computer program may be embodied or encoded on a computer readable data storage medium. For example, the computer readable data storage medium may be a hard disk drive, an optical disk (e.g., CD-ROM, DVD-ROM, or a Blu-ray Disc) or a flash memory storage drive. The computer module 202 may comprise a read/write device 830 such as a floppy disk drive or an optical disk drive for reading from/writing to various memory devices such as optical disks.

The computer system 200 may be specially constructed for the required purposes, or may comprise a general purpose computer or other device selectively activated or reconfigured by a computer program stored in the computer. The algorithms described herein are not inherently related to any particular computer system or other apparatus. Various general purpose machines may be used with programs in accordance with the methods disclosed herein. Alternatively, the construction of more specialized apparatus to perform the required method steps may be appropriate.

For example, the computer program may be stored in a computer readable medium and the software is loaded into the computer system 200 from the computer readable medium. The computer program may then be executed by the computer system 200, in particular, by the processor unit 218. For example, a computer readable medium having such computer program recorded on the computer readable medium is a computer program product. Accordingly, the use of the computer program product in the computer system 200 enables the methods disclosed herein according to embodiments of the present invention to be carried out.

The computer program is not intended to be limited to any particular programming language and implementation thereof. It will be appreciated that a variety of programming languages and coding thereof may be used to implement the methods described herein. Moreover, the computer program is not intended to be limited to any particular control flow. There are many other variants of the computer program, which can use different control flows without departing from the scope of the present invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer program when loaded and executed on such a general-purpose computer effectively results in an apparatus that implements the steps of the preferred method.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

Some portions of the description described hereinbefore are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

The invention may also be implemented as hardware modules. More particular, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the system can also be implemented as a combination of hardware and software modules.

TABLE 1
List of signature genes

| Genes | Name | Other names (Aliases) |
|---|---|---|
| CCL5 | Chemokine (C-C motif) | D17S136E, MGC17164, |
| | ligand 5 | RANTES, SCYA5, SISd, |
| | | TCP228 |
| CCR2 | Chemokine (C-C motif) | hCG 14621, CC-CKR-2, |
| | | CCR2A, CCR2B, CD192, |
| | receptor 2 | CKR2, CKR2A, CKR2B, |
| | | CMKBR2, FLJ78302, |
| | | MCP-1-R, MGC103828, |
| | | MGC111760, MGC168006 |
| CEACAM8 | Carcinoembryonic antigen- | CD66b, CD67, CGM6, |
| | related cell adhesion | NCA-95 |
| | molecule 8 | |
| CXCL10 | Chemokine (C-X-C motif) | C7, IFI10, INP10, IP-10, |
| | ligand 10 | SCYB10, crg-2, gIP-10, |
| | | mob-1 |
| IFNG | Interferon, gamma | IFG, IFI |
| IL6 | Interleukin 6 (interferon, | BSF2, HGF, HSF, IFNB2, |
| | beta 2) | IL-6 |
| NCR3 | Natural cytotoxicity | DAAP-90L16.3, 1C7, |
| | triggering receptor 3 | CD337, LY117, MALS, |
| | | NKp30 |
| TBX21 | T-box 21 | T-PET, T-bet, TBET, |
| | | TBLYM |
| TLR3 | Toll-like receptor 3 | CD283 |
| TNF | Tumor necrosis factor | DADB-70P7.1, DIF, TNF- |
| | | alpha, TNFA, TNFSF2 |
| CCL2 | Chemokine (C-C motif) | GDCF-2, HC11, |
| | ligand 2 | HSMCR30, MCAF, MCP- |
| | | 1, MCP1, MGC9434, |
| | | SCYA2, SMC-CF |
| CD8A | CD8a molecule | CD8, Leu2, MAL, p32 |
| FCGR1A | Fc fragment of IgG, high | RP11-196G18.2, CD64, |
| | affinity Ia, receptor (CD64) | CD64A, FCRI, FLJ18345, |
| | | IGFR1 |
| LTA | Lymphotoxin alpha (TNF | DAMA-25N12.13-004, LT, |
| | superfamily, member 1) | TNFB, TNFSF1 |
| TLR4 | Toll-like receptor 4 | ARMD10, CD284, TOLL, |
| | | hToll |

TABLE 2
Signature genes suitable for SVM algorithm

| Signature 1 | Signature 2 |
|---|---|
| Table 2A | Table 2B |
| CCL5 | CCL5 |
| FCGR1A | CCR2 |
| IFNG | CD8A |
| IL6 | FCGR1A |
| TLR3 | IFNG |
| TLR4 | IL6 |
| TNF | NCR3 |

TABLE 3
Signature genes suitable for KNN algorithm
Signature 1

CCL2
CCL5
CCR2
CD8A
CXCL10
FCGR1A
IL6
NCR3
TBX21
TLR3
TLR4

TABLE 4
Signature genes suitable for NTP algorithm
Signature 1

CCL2
CCR2
TLR3
TLR4
CCL5
IL6
NCR3
TBX21
CXCL10
IFNG
CD8A
FCGR1A
CEACAM8
TNF

TABLE 14
Signature genes suitable for SVM algorithm (Singapore cohort).
Signature 1

CCL2
CD8A
CXCL10
IL6
LTA
NCR3
TBX21
TNF

TABLE 15
Signature genes suitable for SVM algorithm (Hong Kong cohort).
Signature 1

CCR2
CD8A
IL6
LTA
TLR3

TABLE 16
Signature genes suitable for SVM algorithm (Zurich cohort).
Signature 1

CD8A
CXCL10
IL6
TLR3
TLR4

### BRIEF DESCRIPTION OF THE FIGURES

FIG. 1. Combined SVM & KNN prediction method for survival. In both graphs, the upper line in the graph is survival greater than 5 years whilst the lower line is survival less than 5 years.

FIG. 2. The combined SVM & KNN prediction method in predicting Stage I only HCC patients from Sg, HK and Zurich cohort all combined. The upper line in the graph is survival greater than 5 years whilst the lower line is survival less than 5 years.

FIG. 3. NTP prediction method for good vs poor prognosis prediction. In both graphs, the upper line represents good prognosis whilst the lower line represents poor prognosis.

FIG. 4. Prediction of survival of Stage 1 HCC patients using the NTP 14-immune genes prediction method. The upper line in the graph represents good prognosis whilst the lower line represents poor prognosis.

FIG. 5. The NTP 14-immune genes prediction method is able to enhance the prediction value of tumor staging in HCC patients. The upper line in the first graph represents stage I, the middle line stage II, and the bottom line stage III. The upper line in the second graph represents predicted long term survival whilst the lower line represents predicted short term survival.

FIG. 6. The NTP 14-immune genes prediction method is able to predict survival of HCC patients from Stage II and III. In the first graph, the upper line represents stage II, whilst the lower line represents stage III. In the second graph, the upper line represents good prognosis whilst the lower line represents poor prognosis.

FIG. 7. Identification and validation of a 14 immune-gene signature predictive of overall survival in HCC patients. (A) Study design for the identification of a 14 immune-gene signature derived from the training cohort (Sg, n=57) and validated in an independent cohort of patients from HK (n=43) and Zurich (n=55). Heat maps showing the expression profile of the 14 immune genes (Log values) in (B) the training cohort and in (C) the validation cohort. Patients are classified to good or poor prognosis according to prediction by the immune gene signature. FDR: p value of t test adjusted for false discovery rate (multiple testing). Kaplan-Meier analyses for survival in (D) the training cohort, based on leave-one-out cross-validation testing and in (E) the independent validation cohort. Good and poor prognosis refers to the outcome predicted by the immune signature. p=Log rank test p value; HR=hazard ratio and 95% CI=95% confidence interval.

FIG. 8. Superior prognostic power of the 14 immune-gene signature compared to clinical parameters. Kaplan-Meier analyses for survival of (A) stage I patients (n=55, training and validation cohort) according to the immune gene signature accurately predicts patient survival; (B) stage I patients according to grade (n=50); (C) stage II patients (n=46, training and validation cohort) according to the immune gene signature accurately predicts patient survival and (D) stage II patients according to grade (n=45). p=Log rank test p value; HR=hazard ratio and 95% CI=95% confidence interval. (E) The plot shows hazard ratios with 95% confidence interval for subgroup of patients according to clinical and demographic characteristics. Age: Median=61; AFP Conc: Median=20 ng/ml; Tumor size: Median=4.3 cm.

FIG. 9. CXCL10, CCL5 and CCL2 expressions correlate with tumor infiltration by T and NK cells. (A) CXCL10, CCL5 and CCL2 RNA positively correlate with TBX21, CD8A and NCR3 in HCC patients (training and validation cohort, n=172) but not with CD14, CD68, CD19, CD83, IL13, IL17, FOXP3 or IL10. Graphs show p values against Pearson correlation coefficients r. Dotted line shows limit of significance (p<0.05). (B) Representative IF images showing higher density of CXCL10-expressing cells (red) in a tumor sample with high (left) versus low (right) density of infiltrating CD8⁺ and CD56⁺ cells as quantified by IHC. The area in the rectangle is magnified in the left inset. Bar=50 μm; 400× magnification. (C) Correlation of CXCL10 protein expression with the density of CD8⁺ (left) and CD56⁺ (right) immune cells. CXCL10 expression was determined by quantification of CXCL10-labeled area, CD8⁺ and CD56⁺ cell densities were measured by IHC in tumor fields of patient samples (CD8⁺: n=27; CD56⁺: n=19, training and validation cohort). P values and correlation coefficients (r) were calculated using Spearman's correlation test.

FIG. 10. CXCL10, CCL5 and CCL2 are produced by both immune and cancer cells within HCC tumors. (A) qPCR analysis of CXCL10, CCL5 and CCL2 RNA expression in purified tumor cells (Tumor), tumor-infiltrating leukocytes (TIL) and unfractionated HCC nodules (HCC) from freshly resected tumors. The chemokines are expressed in all three compartments. Graphs show means and SD normalized to Tumor. (B) Representative IHC images of CXCL10 (left) and CCL5 (right) showing expression in cells with cancer cell morphology. Bar=50 μm; 200× magnification. (C) Representative IF images showing co-localization of CXCL10 and CD68. Bar=20 μm; 800× magnification. (D) Representative IF images showing co-localization of CCL5 with either CD68 or CD3. Bar=20 μm; 800× magnification.

FIG. 11. The production of CXCL10, CCL5 and CCL2 by HCC cell lines is induced by IFN-γ, TNF-α and TLR3 ligands. ELISA for (A) CXCL10, (B) CCL5 and (C) CCL2 concentration in culture supernatants from SNU-182 HCC cell line 24 hours after stimulation with IFN-γ, TNF-α and/or poly(I:C). Two-tailed Student's unpaired t-test; *p<0.05; **p<0.01; ***p<0.001 compared to unstimulated control. Graphs show means and SD from 3 independent experiments. (D) CXCL10, CCL5 and CCL2 RNA are positively correlated with IFNG, TNF and TLR3 in HCC patients (training and validation cohort, n=172). Graphs show p value against Pearson correlation coefficients r. Dotted lines show limits of significance for r (r=0.15) and p (p=0.05). (E) Transmigration assay with PBMC isolated from healthy donors (n=3) towards unstimulated or stimulated SNU182 cells with IFNγ and poly(I:C) 24 hours prior to transmigration. In blocking experiments, PBMC were pretreated with anti-CXCR3 and anti-CCR5 neutralizing antibodies at 37° C. for 1½ hours. Graphs show means and SEM. P values were calculated using paired t-test against basal transmigration towards unstimulated HCC. *p<0.05.

FIG. 12. High chemokine expression levels, hence tumor infiltration by T and NK cells, are associated with superior patient survival. (A) Representative IHC images of CD8 and CD56 labelling showing higher density of CD8⁺ T and CD56⁺ NK cells in tumor from patients with longer survival (>median survival=3.9 yrs). Bar=50 μm; 200× magnification. (B) Kaplan Meier analysis showing high density of intratumoral CD8⁺ and CD56⁺ immune cells is associated with superior patient survival. A subset of patients was chosen for immune cell quantification by IHC (CD8: n=46, median=74 cells per feed; CD56: n=36, median=42 cells per field; training and validation cohort). p=Log rank p value; HR=hazard ratio and 95% CI=95% confidence interval. (C) CXCL10 (n=26) IF and (D) TLR3 (n=39) IHC staining area positively correlated with the density of activated caspase-3-positive tumor cells. r=Spearman (CXCL10) or Pearson (TLR3) correlation coefficient. (E) Downregulation of CXCL10, CCL5, CCL2 and TLR3 RNA expression in stage II, III and IV (n=114) compared to stage I HCC patients (n=57). Graphs show means and SEM. P values were calculated using two-tailed Mann-Whitney test. *p<0.05; **p<0.01; ***p<0.001. (F) Model showing that the inflammatory cytokines TNF-α and IFN-γ and TLR ligands stimulate cancer cells or macrophages to produce the key chemokines CXCL10, CCL5 and CCL2. These chemokines induce tumor-infiltration by Th1, CD8⁺ T and NK cells which induce cancer cell killing and tumor control. Positive feedback loops result from the production of IFN-γ by activated T or NK cells that further enhance CXCL10 production (see top arrow marked “IFNg”) and CCL5 by activated T cells that can attract more T cells (see right-hand, circular arrow).

FIG. 13 depicts a schematic flowchart illustrating an exemplary method of analysing a patient with HCC according to embodiment(s) of the present invention.

FIG. 14 depicts an exemplary computer system for executing a computer program according to an embodiment of the present invention.

FIG. 15 Validation of NTP analysis by Bootstrapping analysis. (A) Kaplan Meier analyses on training cohort (Sgn=55) and validation cohort (HK, n=43 and Zurich, n=55) based on Bootstrapping analysis. p=log rank p value; 95% CI=95% confidence interval. (B) Kaplan Meier analyses on Stage I (n=55) and Stage II n=46) HCC patients based on Bootstrapping analysis. p=log rank p value; 95% CI=95% confidence interval.

FIG. 16 Lack of predictive power of clinical parameters for overall survival in stage I HCC patients. (A) Overall survival profile for Stage I patients (n=55, both training and validation cohort). (B) Graph shows Kaplan-Meier analysis (log rank p value) Stage I patients according to alfa-fetoprotein (AFP) level (Median 17 ng/ml). 95% CI=95% confidence interval. (C) Graph shows Kaplan-Meier analysis (log rank p value) Stage I patients according to tumor size, cm (Median=4 cm). 95% CI=95% confidence interval. (D) Overall survival profile for Stage II patients (n=46, both training and validation cohort). (E) Graph shows Kaplan-Meier analysis (log rank p value) Stage II patients according to alfa-fetoprotein (AFP) level (Median 30 ng/ml). 95% CI=95% confidence interval. (F) Graph shows Kaplan-Meier analysis (log rank p value) Stage II patients according to tumor size, cm (Median=5 cm). 95% CI=95% confidence interval.

FIG. 17 CXCL10 protein expression correlates with RNA expression and patient survival. (A) Percentage of various immune subsets expressing CXCR3, CCR5 and, CCR2 in PBMC from healthy donors (HD) or HCC patients (HCC pt), non-tumor tissue-infiltrating leukocytes (NIL), or tumor-infiltrating leukocytes (TIL). Analysis performed with flow cytometry. HD PBMC n=10, HCC pt PBMC, TIL and NIL n=5. Blood samples from healthy donors were obtained from the Singapore Health Science Authority blood bank and blood and tumor tissues from HCC patients were obtained from Singapore General Hospital (SGH), all with Ethics Committee approval.

(B) CXCL10 IF staining area correlates with RNA expression analyzed by qPCR(Sgn=13, HK n=8, Zurich n=4). r=Pearson correlation coefficient.
(C) Kaplan meieranalysis of CXCL10 IF staining area shows its correlation with superior patient survival (Sgn=13, HK n=7, Zurich n=5). Median staining area=346 μm2. p=log rank p value; 95% CI=95% confidence interval.
(D) Kaplan meieranalysis of CXCL10 RNA from qPCR shows its correlation with superior patient survival (Sgn=13, HK n=7, Zurich n=5). Median staining area=346 μm2. p=log rank p value; 95% CI=95% confidence interval.

FIG. 18 Lack of association of patient survival with the density of tumor-infiltrating CD68+ macrophages. (A) Representative images of CD68 IHC staining in tumors (red) showing no difference between long versus short survival patients. Bar=50 μm; 200× magnification. Median survival=3.9 yrs. (B) Kaplan Meier analysis on density of CD68+ cells quantified in 10-15 random 100× magnification fields in patient tumor samples (Sgn=20, HK n=8, Zurich n=5) and showed no association with patient survival. Median value for CD68+ cells was 353 cells per field. 95% CI=95% confidence interval

In order that the invention may be readily understood and put into practical effect, the following non-limiting examples are provided.

### Example 1

### How the Invention was Derived and Main Characteristics (Performance)

The invention was derived from modeling of immune gene expression pattern from both Singapore (n=61), Hong Kong (n=56) and Zurich (n=55) cohort of HCC patients using support vector machine (SVM), K-Nearest Neighbor (KNN) as well as Nearest Template Prediction (NTP) computational modeling programmes. Different prediction modeling methods were explored:

1) Singapore HCC cohort as training set and Hong Kong and Zurich HCC cohort (combined) as validation set using three different algorithms & a combination of two algorithms:
a. SVM (< > 5 years survival as cut-off point). The best 2 immune gene signatures are indicated in the table below together with averaged performance for both cohorts: accuracy, specificity [prediction of good prognosis HCC patients (survival years>=5 years)], sensitivity [prediction of poor prognosis HCC patients (survival years<5 years)] & Kaplan Meier survival analysis p value.

TABLE 5

| | SG -> SG | | | | SG -> HK + Zurich | | | |
|---|---|---|---|---|---|---|---|---|
| Genes | accuracy | Specificity | sensitivity | km_pval | accuracy | specificity | sensitivity | km_pval |
| CCL5 | 81.4 | 91.3 | 70 | 0.00002 | 73.5 | 76.9 | 69.0 | 0.0004 |
| FCGR1A | | | | | | | | |
| IFNG IL6 | | | | | | | | |
| TLR3 | | | | | | | | |
| TLR4 TNF | | | | | | | | |
| CCL5 | 76.2 | 87.0 | 63.2 | 0.0035 | 76.3 | 82.2 | 67.7 | 0.00004 |
| CCR2 | | | | | | | | |
| CD8A | | | | | | | | |
| FCGR1A | | | | | | | | |
| IFNG IL6 | | | | | | | | |
| NCR3 | | | | | | | | |

b. KNN (< > 5 years survival as cut-off point). An algorithm similar to SVM & the performance is as good as SVM. The best gene signature with the combination of 11 genes is listed in the table below. The 8 common genes with SVM are CCL5, CCR2, CD8A, FCGR1A, IL6, NCR3, TLR3 and TLR4.

TABLE 6

| | SG->SG | | | | SG->HK | | | |
|---|---|---|---|---|---|---|---|---|
| Genes | accuracy | Specificity | Sensitivety | km_pval | accuracy | specificity | sensitivity | km_pval |
| CCL2 | 78.6 | 91.3 | 63.2 | 0.0014 | 66.7 | 68.2 | 64.5 | 0.0057 |
| CCL5 | | | | | | | | |
| CCR2 | | | | | | | | |
| CD8A | | | | | | | | |
| CXCL10 | | | | | | | | |
| FCGR1A | | | | | | | | |
| IL6 | | | | | | | | |
| NCR3 | | | | | | | | |
| TBX21 | | | | | | | | |
| TLR3 | | | | | | | | |
| TLR4 | | | | | | | | |

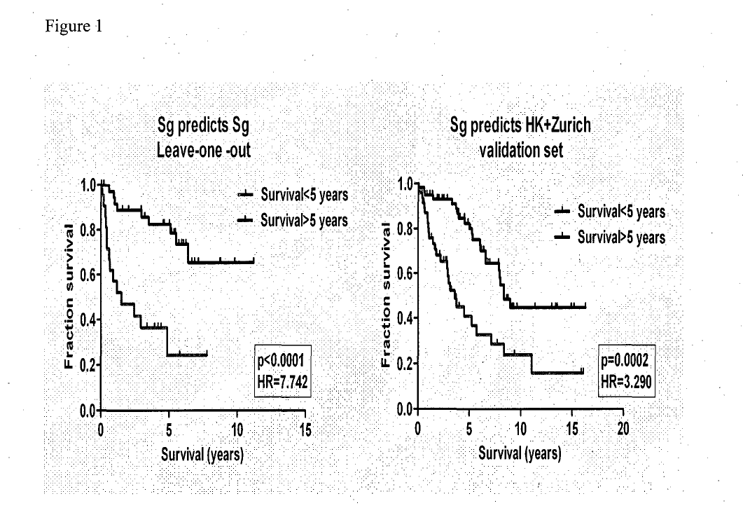

c. SVM combined with KNN. Predictions from the 2 best gene signatures from SVM as well as the 1 best gene signatures from KNN were combined to give a final survival prediction with enhanced accuracy shown in table below & FIG. 1 (see schematic overview of the design). 13 immune genes: CCL2, CCL5, CCR2, CD8A, CXCL10, FCGR1A, IL6, NCR3, TBX21, TLR3, TLR4, IFNG and TNFA were involved in the SVM & KNN combined prediction method. Enhanced accuracy, specificity and sensitivity can be achieved with the combination of 2 independent prediction methods (SVM & KNN).

TABLE 7

| | SG->SG | | | | SG->HK | | | |
|---|---|---|---|---|---|---|---|---|
| | Accuracy | Specificity | sensitivity | km_pval | accuracy | specificity | sensitivity | km_pval |
| Combined | 81.8 | 68.4 | 92.0 | <0.0001 | 75.0 | 71.0 | 77.8 | 0.0002 |
| SVM + | | | | | | | | |
| KNN | | | | | | | | |

Multivariate analysis using tumor stage, tumor size and the combined SVM & KNN prediction method shows that the prediction method is an independent predictor of survival with p value as good as tumor stage as shown in the table below:

TABLE 8

| | Univariate analysis^{a} | | Multivariate Analysis^{b} | |
|---|---|---|---|---|
| | Hazard Ratio | | Hazard Ratio | |
| Variable | (95% CI^{c}) | p value | (95% CI) | p value |
| Sg, training set, n = 61 | | | | |
| SVM + KNN | 7.742 (2.94-20.39) | <0.0001* | 4.699 (1.955-11.296) | 0.0005* |
| TMN Stage (I/II/III) | n.a | 0.0015* | 1.963 (1.253-3.076) | 0.0033* |
| Tumor size, cm (>6 cm) | 0.7433 (0.2486-2.222) | 0.5955 | n.a. | n.a. |
| HK + Zurich, validation set, | | | | |
| n = 111 | | | | |
| SVM + KNN | 3.29 (1.773-6.106) | 0.0002* | 2.114 (1.2127-3.684) | 0.0083* |
| TMN Stage (I/II/III/IV) | n.a. | <0.0001* | 1.876 (1.2752-2.758) | 0.0014* |
| Tumor size, cm (>6 cm) | 1.935 (1.068-3.507) | 0.0295* | 1.263 (0.6659-2.395) | 0.4748 |

^{a}Univariate analysis, Kaplan Meier.
^{b}Multivariate analysis, Cox proportional hazards regression.
^{C}95% CI, 95% confidence interval.
*Significant.

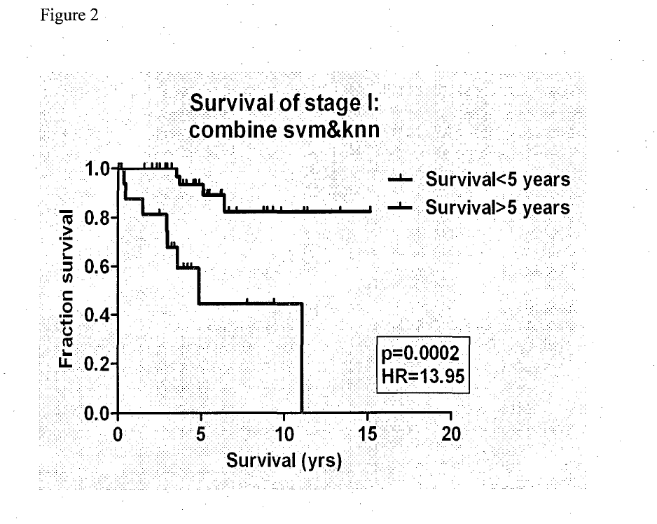

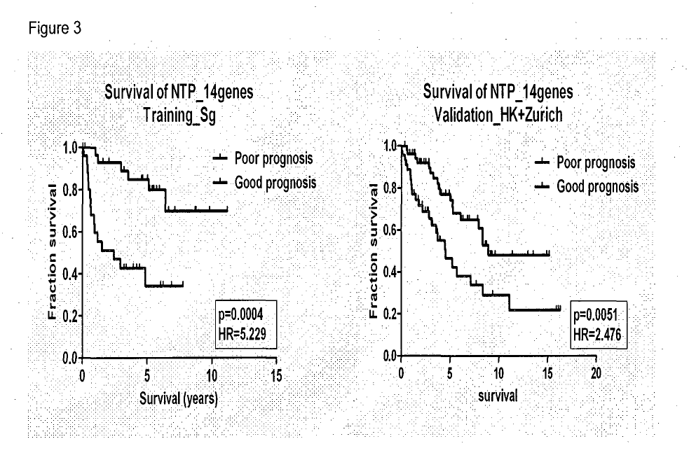

The combined SVM & KNN prediction method also performs well in predicting Stage I only HCC patients from Sg, HK and Zurich cohort all combined n=55 (KM graph shown in FIG. 2), showing its superiority in predicting survival of early stage patients.

d. NTP. This algorithm creates a template for good vs poor prognosis prediction which is independent of the definition of survival cut-off, and therefore it is not affected by different median follow-up years in different cohorts. For more details please refer to Hoshida Y (2010) Nearest Template Prediction: A Single-Sample-Based Flexible Class Prediction with Confidence Assessment. PLoS ONE 5(11): el5543. doi:10.1371/journal.pone.0015543, content of which is incorporated herein by reference. Training using 14 immune genes: CCL2, CCR2, TLR3, TLR4, CCL5, IL6, NCR3, TBX21, CXCL10, IFNG, CD8A, FCGR1A, CEACAM8 and TNF with Singapore cohort (n=57) and independently validated in HK (n=43) and Zurich (n=55) patients. KM p value=0.0004; HR=5.23 for Singapore cohort (training: leave-one-out cross validation) and KM p value=0.0051; HR=2.48 for HK+Zurich cohort (independent validation cohort):

Multivariate analysis using tumor stage and the NTP 14-immune genes signature shows that the prediction method is an independent predictor of survival with p value as good as tumor stage as shown in the table below:

TABLE 9

| | Univariate analysis^{a} | | Multivariate Analysis^{b} | |
|---|---|---|---|---|
| | Hazard Ratio | | Hazard Ratio | |
| Variable | (95% CI^{c}) | p value | (95% CI) | p value |
| Sg, training set, n = 61 | | | | |
| SVM + KNN | 5.229 (2.104-13.00) | 0.0004* | 3.797 (1.419-10.159) | 0.0079* |
| TMN Stage (I/II/III) | n.a | 0.0015* | 1.854 (1.158-2.968) | 0.0102* |
| Tumor size, cm (>6 cm) | 0.7433 (0.2486-2.222) | 0.5955 | n.a. | n.a. |
| HK + Zurich, validation | | | | |
| set, n = 111 | | | | |
| SVM + KNN | 2.476 (1.313-4.669) | 0.0051* | 2.007 (1.062-3.794) | 0.032* |
| TMN Stage (I/II/III/IV) | n.a. | <0.0001* | 1.594 (1.080-2.351) | 0.0188* |
| Tumor size, cm (>6 cm) | 1.825 (0.931-3.579) | 0.0797 | n.a. | n.a. |

^{a}Univariate analysis, Kaplan Meier.
^{b}Multivariate analysis, Cox proportional hazards regression.
^{c}95% CI, 95% confidence interval.
*Significant.

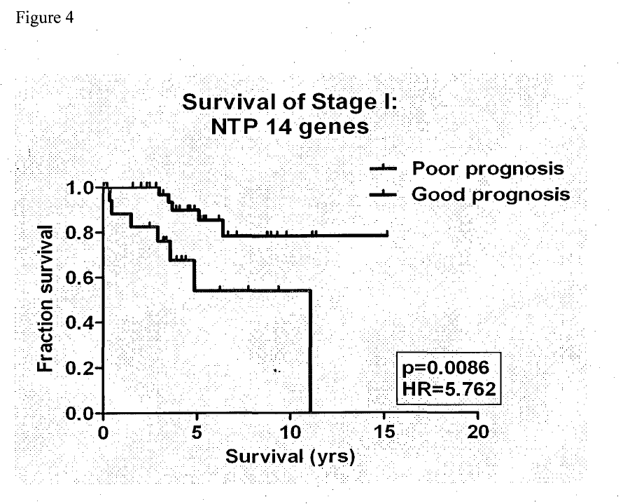

Most importantly the NTP 14-immune genes prediction method which has been blindly and independently validated on HK and Zurich patients also performs well in predicting Stage I only HCC patients from all regions: Sg, HK and Zurich cohort combined n=55 (KM graph shown in FIG. 4). This shows its superiority in predicting survival of early stage patients.

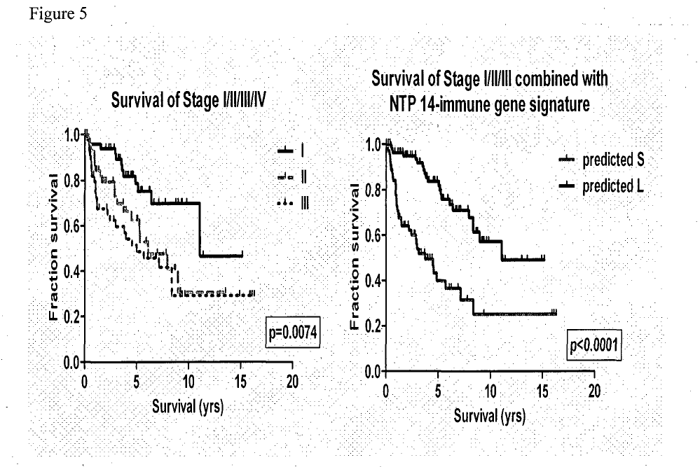

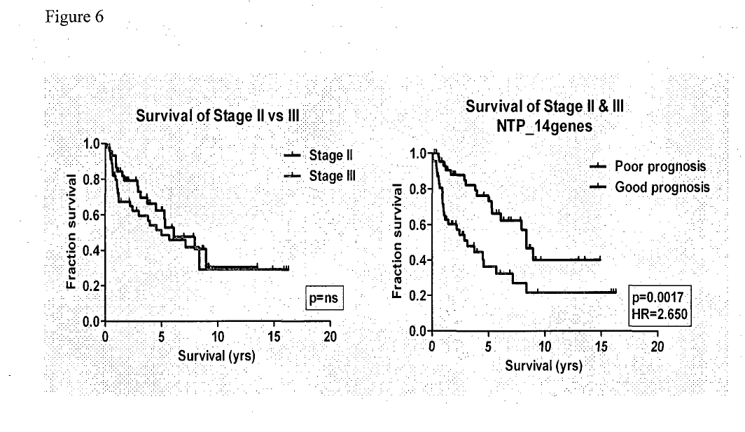

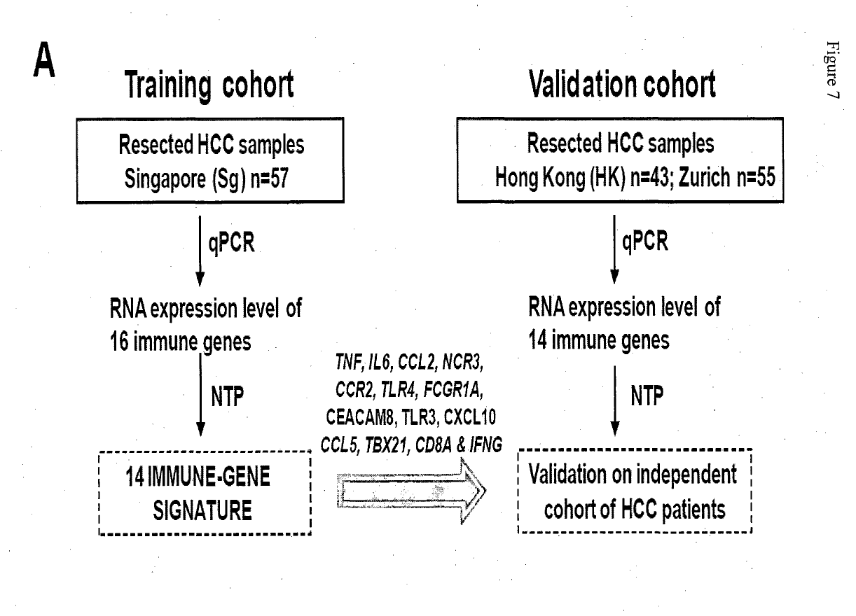

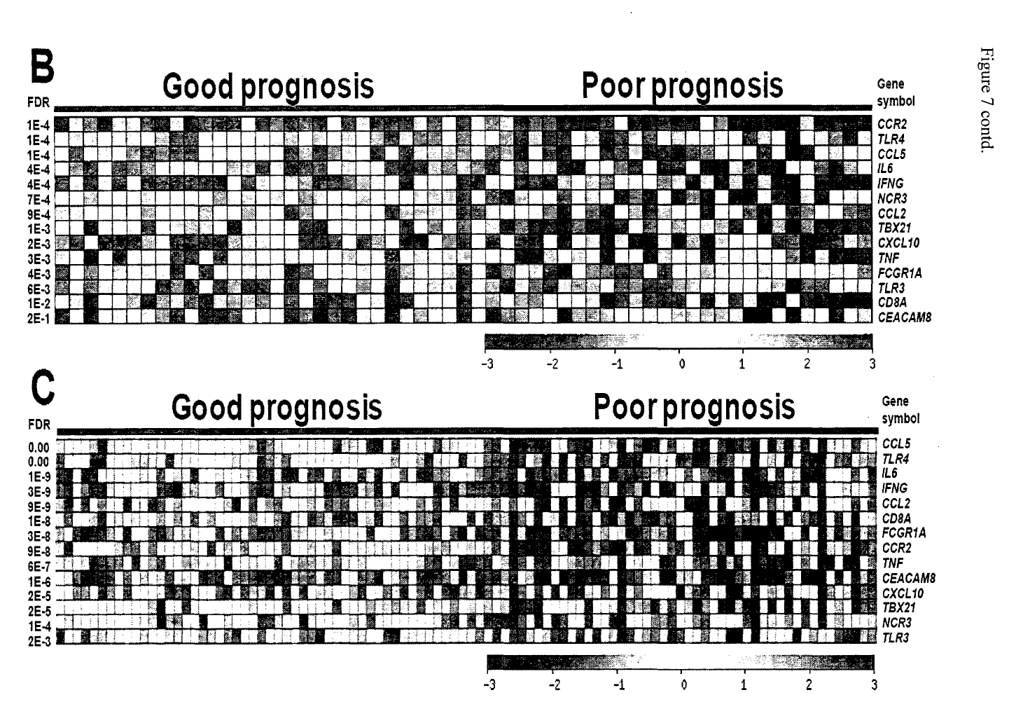

2) The immune gene signatures can enhance or even be superior to the prediction value of tumor staging:
a. The NTP 14-immune genes prediction method is able to enhance the prediction value of tumor staging in HCC patients. KM graphs are shown in FIG. 5: Total patients n=147 (Sg n=57, HK n=37, Zurich n=53): Stage I/II/III-KM p value=0.0074 vs Stage I/II/III combined with the 14-immune gene NTP prediction method-KM p value<0.0001.
b. The NTP 14-immune genes prediction method is able to predict survival of HCC patients from Stage II & III which usually have very similar survival profiles (p=ns). This is very useful for HCC patients from Stage II or III where tumor staging alone is not able to segregate patients into good or poor prognosis. KM graphs are shown in FIG. 6 for all Stage II & III patients from Sg, HK and Zurich cohort combined (n=92):
3) The best immune gene signature from individual cohorts (Sg or HK) can be used to predict prognosis within the same cohort using SVM (< > 5 years):
a. Signature derived from the Singapore cohort to predict prognosis of a Singapore HCC patient. The best gene signature is CCL2, CD8A, CXCL10, IL6, LTA, NCR3, TBX21 and TNF: with accuracy=86.05%, specificity=86.96%, sensitivity=85% & KM p value=0.000089.
b. Signature derived from the Hong Kong cohort to predict prognosis of a Hong Kong HCC patient. The best gene signature is CCR2, CD8A, IL6, LTA and TLR3: with accuracy=80.49%, specificity=100%, sensitivity=42.86% & KM p value=0.00000051.
c. Signature derived from the Zurich cohort to predict prognosis of a Zurich HCC patient.

The best gene signature is CD8A, CXCL10, IL6, TLR3 and TLR4: with accuracy=89.29%, specificity=83.33%, sensitivity=93.75% & KM p value=0.0011.

### Example 2

### How the Invention May be Used

A fragment of resected tumor or biopsy will be subjected to total RNA extraction, e.g. by using Trizol (Invitrogen) & RNA will be converted to DNA such as by using Taqman Reverse Transcriptase reagent (Applied Biosystems). The level of expression of between the following immune genes: CCL5, CCR2, CEACAM8, CXCL10, IFNG, IL6, NCR3, TBX21, TLR3, CD8A, LTA, TNF, FCGR1, CCL2 and TLR4 will be analysed by quantitative PCR, optionally using iTaq SYBR Green Supermix with ROX (Bio-Rad Laboratories). The primers sequences are listed in Chew et al. Journal of Hepatology 2010, 52:370-9. The level of expression of the immune genes will be normalized to the house-keeping gene ACTB e.g. using MxPro software (Stratagene). Additional normalization with the median value of each particular gene according to training cohort (Sg cohort) will also be done (See Table 10 below for the median values of each gene from Sg as the training cohort). After which, the prediction models (algorithms) will be applied to the values obtained. One can choose to use:
1. The model from SVM, KNN (< > 5 years) or NTP using Sg as training set & Hong Kong and Zurich as validation set for any HCC patient from any region or;
2. The combined SVM & KNN (< > 5 years) prediction method using Sg as training set & Hong Kong and Zurich as validation set for any HCC patient from any region or,
3. The model designed for each individual cohort of the patient is from either Sg,HK or Zurich for more accurate prediction.
4. The NTP 14-immune genes prediction method in combination with staging information.
5. The NTP 14-immune genes or the combined SVM & KNN (< > 5 years) prediction method for any Stage I HCC patient.

SVM or KNN (< > 5 years) provides prediction of prognosis with information regarding survival (longer or shorter than 5 years) whereas NTP provides only a general good or poor prognosis profile.

TABLE 10

| No. | Genes | Median value (as normalized to ACTB) |
|---|---|---|
| 1 | CCL5 | 1.76E−02 |
| 2 | CCL2 | 1.06E−02 |
| 3 | CCR2 | 9.45E−05 |
| 4 | CEACAM8 | 1.13E−04 |
| 5 | CXCL10 | 1.65E−03 |
| 6 | IFNG | 1.98E−05 |
| 7 | IL6 | 1.55E−03 |
| 8 | NCR3 | 1.17E−03 |
| 9 | TBX21 | 1.29E−03 |
| 10 | TLR3 | 3.80E−04 |
| 11 | TLR4 | 3.79E−04 |
| 12 | TNF | 5.98E−04 |
| 13 | CD8A | 2.95E−03 |
| 14 | FCGR1A | 1.39E−03 |

### Example 3

### Summary

Objective:

Hepatocellular carcinoma (HCC) is a heterogeneous disease with poor prognosis and limited methods for predicting patient survival. The nature of the immune cells that infiltrate tumors is known to impact clinical outcome. However, the molecular events that regulate this infiltration require further understanding. Here it is investigated how immune genes expressed in the tumor microenvironment predict disease progression.

Design:

Using quantitative polymerase chain reaction, the expression of 14 immune genes in resected tumor tissues from 57 Singaporean patients was analyzed. The nearest-template prediction method was used to derive and test a prognostic signature from this training cohort. The signature was then validated in an independent cohort of 98 patients from Hong Kong and Zurich. Intratumoral components expressing these critical immune genes were identified by in situ labeling. Regulation of these genes was analyzed in vitro using the HCC cell line SNU-182.

Results:

The identified 14 immune-gene signature predicts patient survival in both the training cohort (p=0.0004 and hazard ratio=5.2) and validation cohort (p=0.0051 and hazard ratio=2.5) irrespective of patient ethnicity and disease etiology. Importantly, it predicts the survival of patients with early disease (Stage I and II), for whom classical clinical parameters provide limited information. The lack of predictive power in late disease stages III and IV emphasizes that a protective immune microenvironment has to be established early in order to impact disease progression significantly. This signature includes the chemokine genes CXCL10, CCL5 and, CCL2, whose expression correlates with markers of Th1, CD8⁺ T and, NK cells. Inflammatory cytokines (TNF-α, IFN-γ and TLR3 ligands stimulate intratumoral production of these chemokines which drive tumor infiltration by T and NK cells, leading to enhanced cancer cell death.

Conclusion:

A 14 immune-gene signature, which identifies molecular cues driving tumor infiltration by lymphocytes, accurately predicts HCC patient survival especially in early disease. The gene signature was predictive of HCC patient survival in both the training cohort from Singapore (n=57; p=0.0004 and hazard ratio=5.2) and validation cohort from Hong Kong and Zurich (n=98; p=0.0051 and hazard ratio=2.5) irrespective of patient ethnicity and disease etiology.

### Introduction

It is now recognized that cancer progression is regulated by both cancer cell-intrinsic and micro-environmental factors. Among the latter, the nature and localization of immune cells infiltrating the tumor play a central role. While tumor infiltration by myeloid cells is often associated with a poor prognosis, the presence of Th1 or cytotoxic T cells correlates with a reduced risk of relapse in several cancers.

It was previously found that a pro-inflammatory tumor microenvironment correlates with prolonged survival in a cohort of Singaporean HCC patients [16]. In the current study, a 14 immune-gene signature was identified which was able to predict patient survival from this cohort and it was validated it in an independent cohort of patients from Hong Kong and Zurich. By combining transcriptome analysis, in situ labeling and in vitro experiments, the cellular sources of the molecules corresponding to the gene signature were identified. This approach revealed 1) a paracrine loop involving CXCL10, TLR3, TNF-α and IFN-γ and 2) an autocrine loop controlling CCL5 production. These two loops shape the immune milieu and recruit a potent anti-tumoral lymphoid infiltrate to the tumor of patients with longer survival. This study shows that features derived from the tumor immune microenvironment are of general predictive value irrespective of HCC heterogeneity. Importantly, they determine the clinical outcome of patients with early stages HCC for whom clinical parameters provide limited survival information. The lack of predictive power in late stages shows, for the first time in HCC, that the protective immune microenvironment has to be established early to promote long-term survival.

### Materials and Methods

Patients.

172 resected HCC mRNA samples (one from each patient) were obtained from the National Cancer Centre (NCC), Singapore, Sg (n=61), the Queen Mary Hospital (QMH), Hong Kong, HK (n=56) and the University Hospital Zurich, Switzerland (n=55). All samples were obtained with Ethics Committee approval from patients who underwent curative resection from 1991 to 2009. After censoring patients with poor-quality gene expression profiles, data from Singapore patients (n=57) were used as a training cohort to derive and test the survival prediction model, while Hong Kong (n=43) and Zurich (n=55) patients were used as an independent validation cohorts. A total of 49 paraffin-embedded HCC samples (Sg, n=20; HK, n=23; Zurich, n=6) were obtained for immunohistochemistry or immunofluorescence labeling.

Clinical and demographic characteristics of the training and validation cohorts are summarized in Table 11.

Analysis of Gene Expression.

Quantitative polymerase chain reaction (qPCR) analysis was performed on a total of 172 resected HCC mRNA samples. Primers were designed using Primer3 and qPCR was performed using iTaq SYBR Green Supermix with ROX (Bio-Rad Laboratories), as described previously [16]. Sixteen immune genes were selected for expression analysis. Two of the genes, LTA and CCL22, were omitted from the gene-list due to very low/undetectable expression in many of the validation cohorts. Relative gene expression level was calculated by normalization to the housekeeping gene ACTB using MxPro software (Stratagene).

Statistical Analyses.

Survival prediction was performed using the nearest template prediction (NTP) method. The Cox score for each gene, which reflects the correlation between gene expression level and patient survival, was calculated as described previously [10]. The prognosis prediction for each sample was made based on the proximity of its gene expression level to either of the templates of poor or good prognosis as defined by the vectors of weighted Cox scores. The survival predictor was evaluated in the training cohort (Sg, n=57) using a leave-one-out cross-validation, and tested on the independent validation cohort (HK, n=43 and Zurich, n=55). NTP was also validated by Bootstrap method as described previously. [17] Two-class differential expression analysis was performed using GEPAS version 4.0 (http://gepas.bioinfo.cipf.es/).

Kaplan-Meier univariate survival analysis was performed using GraphPad Prism. Survival prediction is classified as “good prognosis” or “poor prognosis” according to the gene signature or as “Low” or “High” as compared to the median of the relevant parameters. Patients who are still alive at last follow-up or are deceased due to causes unrelated to HCC were censored. Reported p values are obtained from Log-rank (Mantel-Cox) test. Multivariate analysis by Cox proportional hazards model was used to examine the gene signature in the context of clinical variables. The NTP method and multivariate analyses were performed with the use of R statistical package (www.r-project.org).

Immunohistochemistry and Immunofluorescence.

Immunohistochemistry (IHC) or immunofluorescence (IF) labeling were performed on paraffin-embedded HCC samples as described before [16]. IHC images were captured with an Olympus DP20 camera attached to a CX31 microscope. For IF an Olympus FlourView FV1000 confocal microscope was used. Quantification of positive cells was performed with ImagePro Software from 5-10 random fields at 100× magnification for IHC, or 10-15 random fields at 200× magnification for IF. The average value from all quantified fields was determined for each patient. Statistical analysis was performed with GraphPad Prism.

Isolation of Peripheral Blood Mononuclear Cells and Tumor-Infiltrating Leukocytes.

Tumor tissues from HCC patients (n=3) were obtained from Singapore General Hospital (SGH) with Ethics Committee approval. Tissues were homogenized using Dispomix® Drive (Xiril AG). Tumor (T) and tumor-infiltrating leukocytes (TIL) were separated by a series of low speed centrifugations and filtration through a 100 μm filter (Millipore) to remove large debris. 1×10⁶ cells were resuspended in Trizol (Invitrogen) and RNA was converted to cDNA using Taqman Reverse Transcriptase reagent (Applied Biosystems) for qPCR analysis. Fraction purity assessed by flow cytometry was around 90%.

In Vitro Chemokine Production and Transwell Migration Assays.

The HCC cell line SNU-182 was obtained from the Korean Cell bank and cultured in complete RPMI medium. Cells were treated with 100 U/ml IFN-γ (ImmunoTools), 10 ng/ml TNF-α, 50 μg/ml poly I:C (InvivoGen) or with a combination of IFN-γ and TNF-α, or IFN-γ and polyI:C. After 24 hours, culture supernatants were collected for ELISA and cells were harvested for RNA isolation. RNA isolation, cDNA conversion and qPCR for CXCL10, CCL5 and CCL2 were performed as described above. ELISAs were performed to detect CXCL10, CCL5 and CCL2 using kits from R&D Systems (CXCL10 and CCL5) and eBiosciences (CCL2) according to the manufacturers' instructions. Absorbance intensity was analysed using a Tecan microplate reader.

For transwell migration assay, SNU182 cells unstimulated or stimulated with IFN-γ and poly(I:C) as described above were seeded into 24-well plates. After 24 hours, 1×10⁶ PBMC from healthy donors (n=3) untreated or pretreated with anti-CXCR3 (25 μg/ml; clone 106, BD Pharmingen) or anti-CCR5 (10 μg/ml; clone 2D7, BD Phanningen) neutralizing antibodies at 37° C. for 1½ hours were added onto the transwell filter inserts (3 μm pore size, BD Falcon). Transmigration was assessed after 3 hours.

### Results

### Identification and Validation of an Immune Gene Signature Predicting Overall Survival of HCC Patients

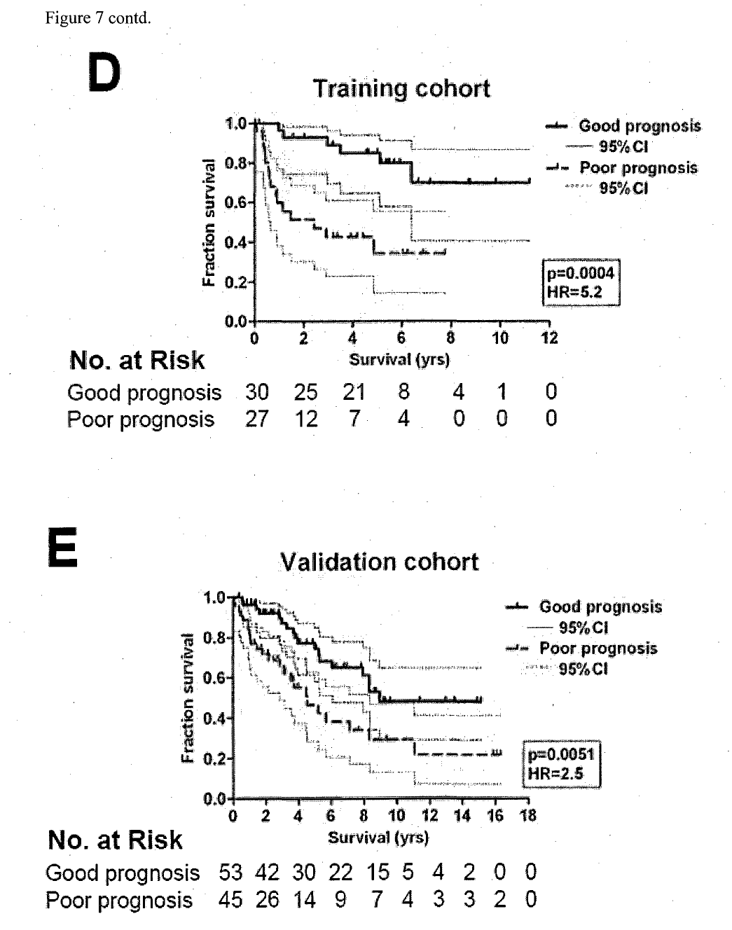

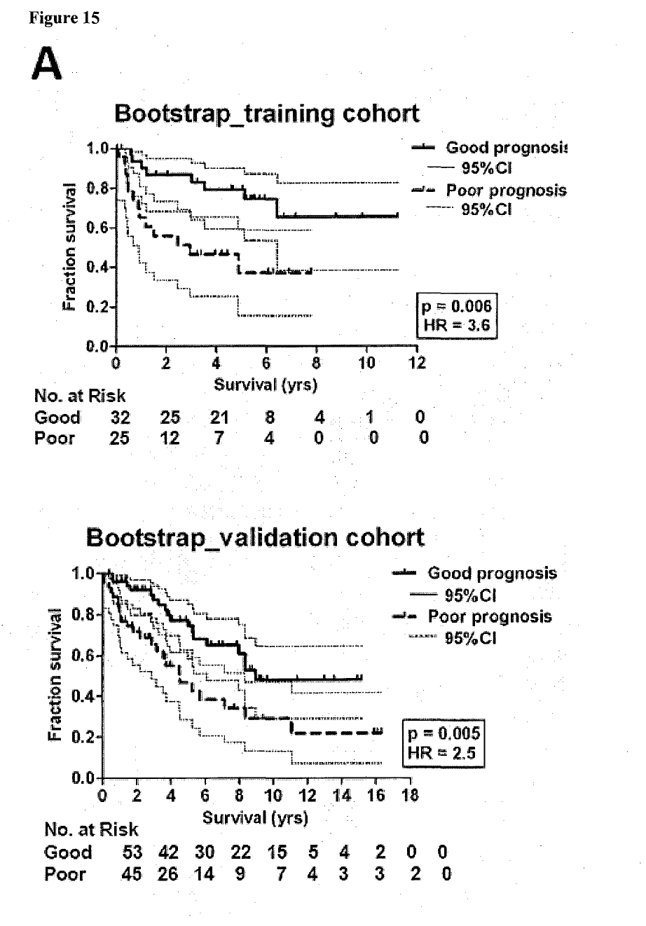

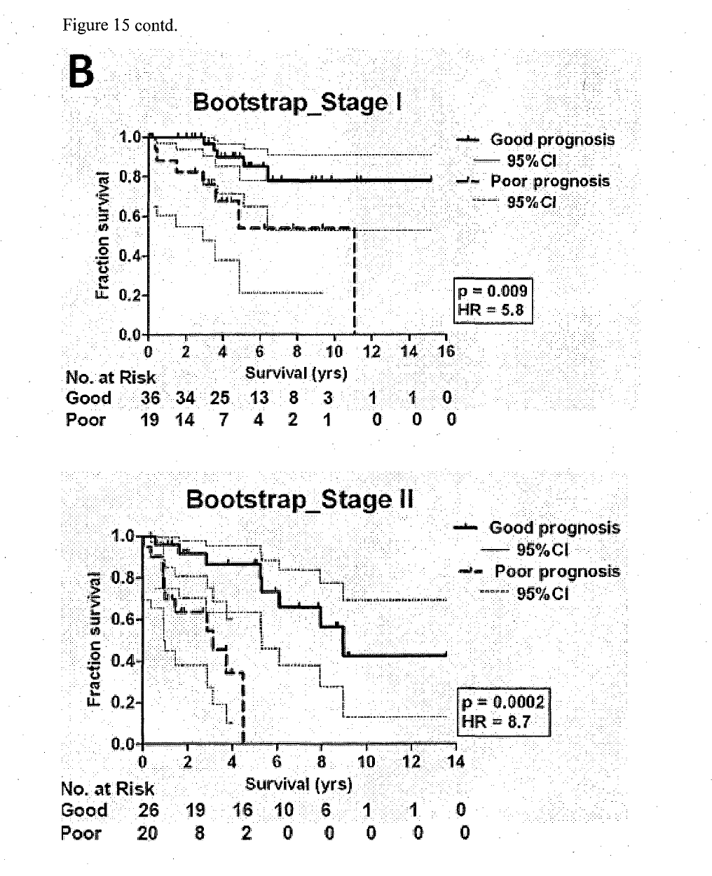

The expression profile of 49 immune-related genes in 61 resected HCC tumor samples from Singapore was previously characterized and 11 immune genes were found whose expression was associated with superior patient survival [16]. In the current study, the RNA expression of 14 immune genes was analyzed: TNF, IL6, CCL2, NCR3, CCR2, TLR4, FCGR1A, CEACAM8, TLR3, CXCL10, CCL5, TBX21, CD8A and IFNG. Nearest template prediction (NTP) was used to identify and cross-validate (by leave-one-out method) a 14 immune-gene signature predictive of overall survival in 57 Singaporean HCC patients with resectable HCC (as a training cohort). The NTP method was chosen because it allows independent prediction for each sample and is less sensitive to differences in sample processing and analysis [18]. The signature was then validated in an independent cohort of patients from Hong Kong (n=43) and Zurich (n=55) (FIG. 7A). Bootstrapping analysis also showed similar results (FIG. 15).

In general, the 14 immune genes display higher expression in patients with good prognosis in both the training (FIG. 7B) and the validation cohort (FIG. 7C). The relative importance of each gene was assessed using its cox score (Table 13).

TABLE 13
The list of 14 immune genes in order of decreasing importance
based on the cox score of each gene in training cohort, IL-6 being
the most important and CEACAM8 the least. Note that a negative
value represents a positive correlation with survival.

| Gene | cox score |
|---|---|
| IL6 | −2.683275671 |
| TLR4 | −2.305472414 |
| NCR3 | −2.224820683 |
| CCL2 | −2.181026188 |
| CXCL10 | −1.712844345 |
| CCR2 | −1.709388501 |
| CCL5 | −1.601773463 |
| TNF | −1.566062324 |
| FCGR1A | −1.154882937 |
| TLR3 | −0.538128834 |
| IFNG | −0.348678936 |
| TBX21 | −0.223167598 |
| CD8A | −0.095256421 |
| CEACAM8 | 0.275850045 |

Despite the differences in patient ethnicity and disease stage (Table 11), the herein presented 14-gene signature accurately predicts patient survival in both the training cohort (p=0.0004 and hazard ratio=5.2; FIG. 7D) and the validation cohort (p=0.0051 and hazard ratio=2.5; FIG. 1E). Multivariate analysis showed that this gene signature is an independent predictor of survival with regard to stage or six other clinical parameters (Table 12). Strikingly, when stage IV patients were excluded, the immune signature was the only predictor of survival (Table 12).

TABLE 11
Comparison of clinical and demographic characteristics of HCC
patients in training (Sg) and validation (HK + Zurich) cohorts

| | | Training cohort | | Validation cohort | | |
|---|---|---|---|---|---|---|
| Variables | | (n = 57) | | (n = 98) | | p-value |
| Sex, F/M | Number | 7/50 | (12/88) | 21/77 | (21/79) | ns* |
| | (percent) | | | | | |
| Age, years | Median | 59 | (31-84) | 60 | (20-83) | ns@ |
| | (Range) | | | | | |
| Race, | Number | 57/0 | (100/0) | 46/52 | (47/53) | <0.0001* |
| Asian/European | (percent) | | | | | |
| Viral status, Non- | Number | 12/43 | (21/75) | 32/66 | (33/67) | ns* |
| infected/HepB, C, D | (percent) | | | | | |
| Grade, 1+2/3+4 | Number | 33/21 | (58/37) | 61/24 | (62/24) | ns^{$} |
| | (percent) | | | | | |
| TMN Staging, I/ | Number | 34/23 | (60/40) | 21/77 | (21/79) | <0.0001* |
| II + III + IV | (percent) | | | | | |
| α-fetoprotein, ng/ml | Median | 19 | (1.5−>70,000) | 50 | (1-468,600) | ns@ |
| | (Range) | | | | | |
| Tumor size, cm | Median | 6 | (0.7-23) | 5 | (1.2-23.5) | ns@ |
| | (Range) | | | | | |
| Survival, years | Median | 3.94 | (0.9/5.5) | 3.8 | (1.6/7.8) | ns# |
| | (25^{th}/75^{th} %) | | | | | |

*Fisher's exact test
#Kaplan-Meier
@Mann-Whitney
^{$}good/poor differentiation; different classification system for HK cohort

TABLE 12
Multivariate analysis of the 14 immune-gene signature

| | Univariate analysis^{a} | | Multivariate Analysis^{b} | |
|---|---|---|---|---|
| | Hazard Ratio | | Hazard Ratio | |
| Variable | (95% CI^{c}) | pval | (95% CI^{c}) | pval |
| Training cohort | | | | |
| All patients; n = 57 | | | | |
| Immune gene signature | 4.9 (1.9-12.8) | 0.001* | 3.8 (1.4-10.1) | 0.008* |
| TMN Stage (I/II/III) | 2.2 (1.4-3.5) | 0.001* | 1.9 (1.2-3.0) | 0.010* |
| Validation cohort | | | | |
| All patients; n = 98 | | | | |
| Immune gene signature | 2.3 (1.3-4.3) | 0.007* | 2.0 (1.1-3.8) | 0.032* |
| TMN Stage (I/II/III/IV) | 1.8 (1.2-2.6) | 0.003* | 1.6 (1.1-2.4) | 0.019* |
| Stage I/II/III patients; n = 91 | | | | |
| Immune gene signature | 2.4 (1.2-4.7) | 0.009* | 2.2 (1.1-4.4) | 0.022* |
| TMN Stage (I/II/III) | 1.4 (0.9-2.2) | 0.120 | 1.2 (0.8-1.9) | 0.331 |
| Training + validation cohort | | | | |
| All patients; n = 155 | | | | |
| Immune gene signature | 3.0 (1.8-5.1) | 2.18E-05 | 2.7 (1.4-5.2) | 0.004* |
| Grade (1/2/3/4) | 1.4 (0.9-2.0) | 0.137 | 1.4 (0.9-2.4) | 0.157 |
| TMN stage (I/II/III/IV) | 1.8 (1.4-2.4) | 2.14E-05 | 1.8 (1.2-2.8) | 0.005* |
| Tumor size (<median/≧median) | 1.4 (0.8-2.5) | 0.253 | 0.6 (0.3-1.2) | 0.158 |
| AFP (<median/≧median) | 1.4 (0.8-2.3) | 0.207 | 1.2 (0.6-2.2) | 0.649 |
| Age (<median/≧median) | 1.4 (0.8-2.2) | 0.236 | 1.6 (0.9-3.0) | 0.144 |

Abbreviations: pval, p value;
^{a}Univariate analysis, Cox proportional hazard regression.
^{b}Multivariate analysis, Cox proportional hazard regression.
^{c}95% CI, 95% confidence interval.
*Significant (p < 0.05).
Median values, tumor size = 5.4 cm; AFP = 25 ng/ml; Age = 60.

### Superior Predictive Power of the 14 Immune-Gene Signature in Early Stage Patients

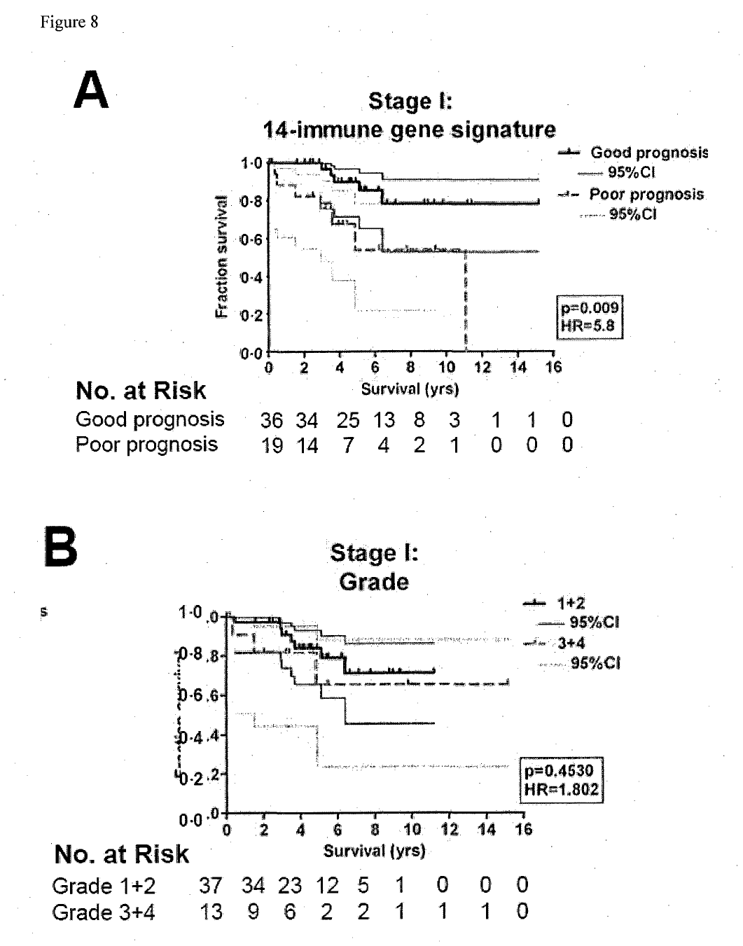

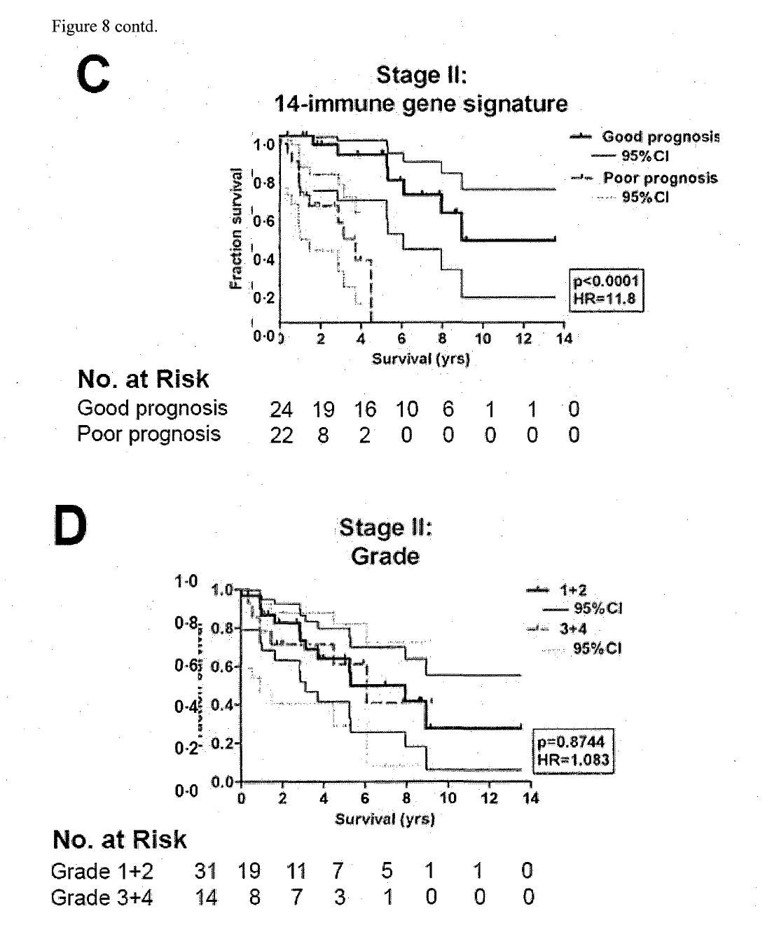

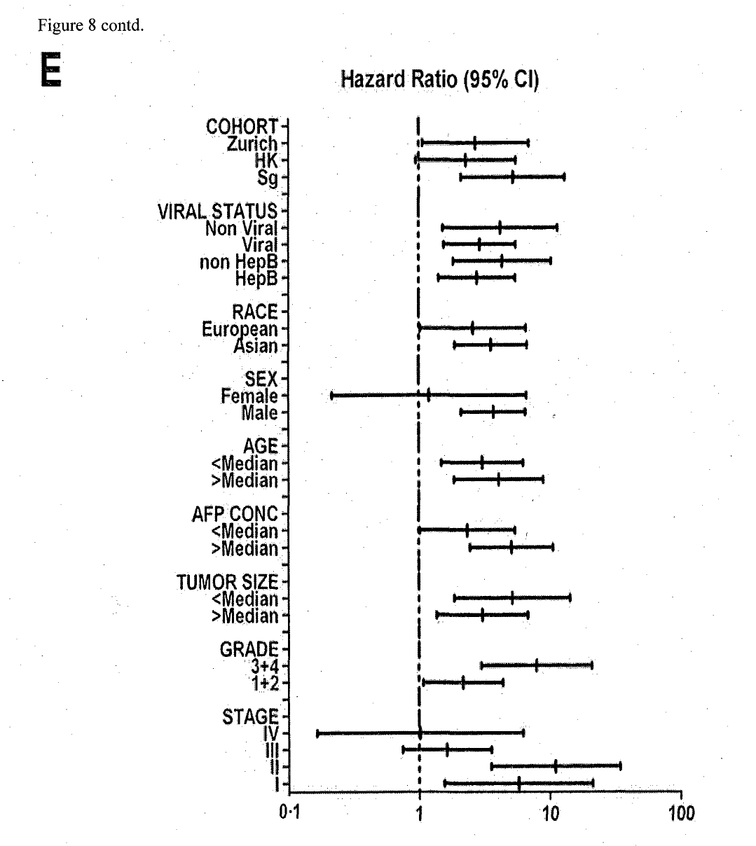

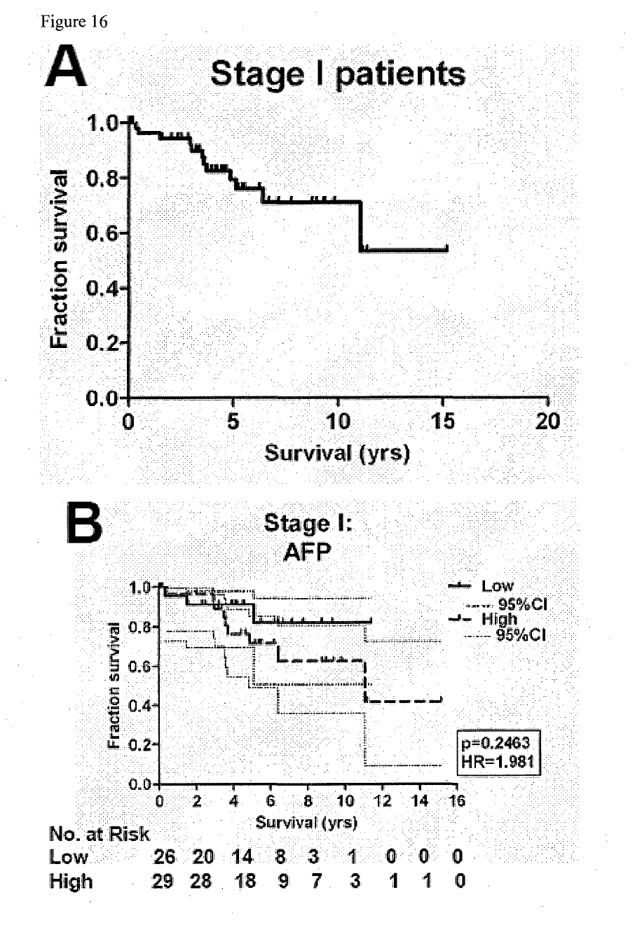

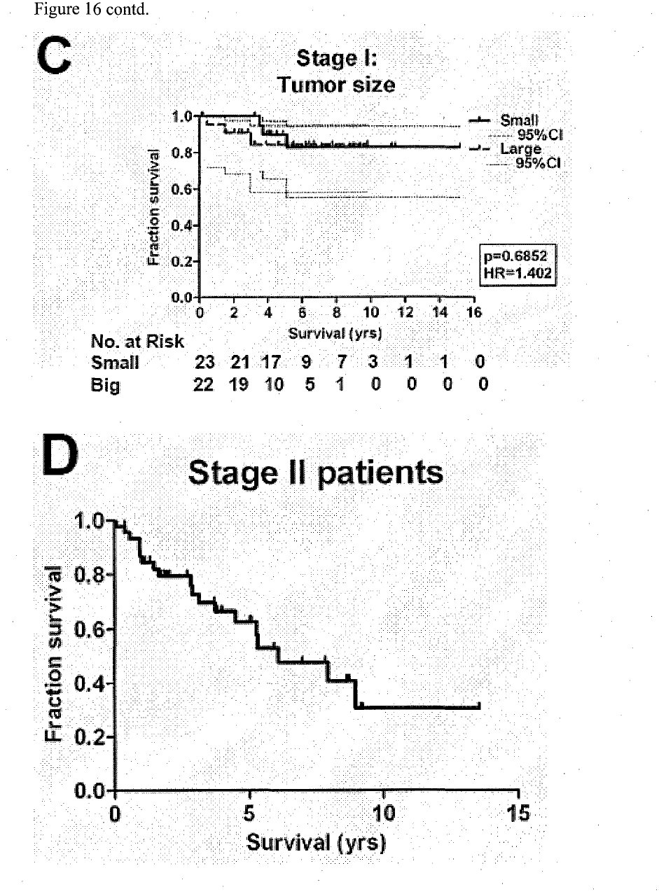

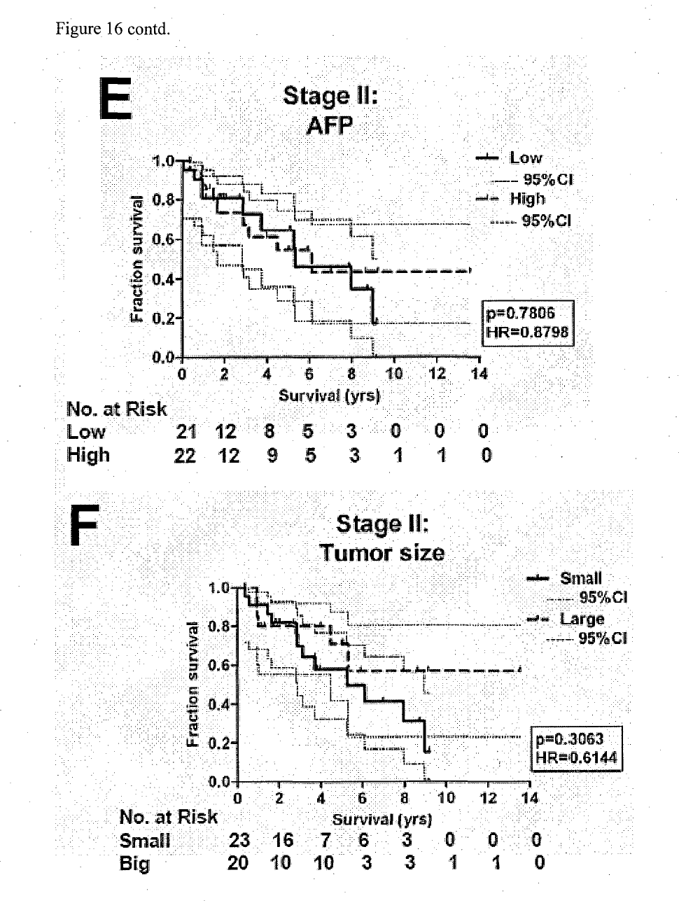

In the Singapore cohort, 60% of patients presented with stage I disease at diagnosis (Table 11). The performance of the identified immune signature in patients with early (stage I and II) disease was therefore measured and compared with clinical parameters generally used for prognosis of such patients. First, it was noted that stage I (n=55) and II (n=46) patients (from both the training and validation cohorts) present a wide range of survival times, from a few months to more than 15 years (FIG. 16). The immune signature accurately predicted the overall survival of these patients in Kaplan-Meier analyses (Stage I: p=0.009, hazard ratio=5.8; Stage II: p<0.0001, hazard ratio=11.8) (FIGS. 8A and 8C). On the contrary, clinical parameters such as grade (FIGS. 8B and D), serum alpha-fetoprotein (AFP) concentration or tumor size (FIG. 16) did not predict overall survival of these patients. Similar results were obtained from Bootstrapping analysis (FIG. 15).

The predictive power of the 14-gene signature was also tested in various subgroups of patients (FIG. 8E). Interestingly, it did not predict the survival of stage III or IV patients. Therefore, the immune signature allows a robust and reliable prediction of overall survival in early HCC patients for whom classical clinical parameters are not significant.

CXCL10, CCL5 and CCL2 Expression Correlates with Intratumoral Infiltration of Th1, CD8⁺ T and NK Cells

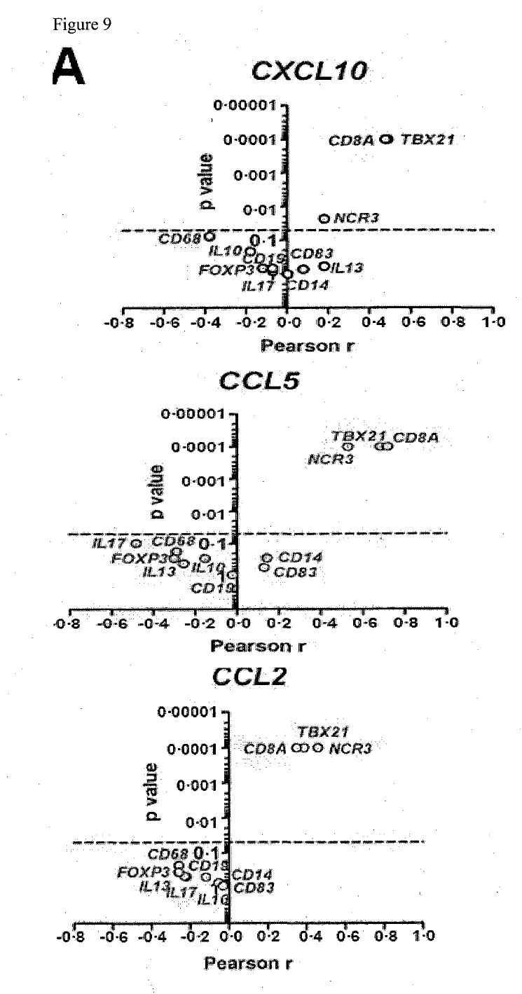

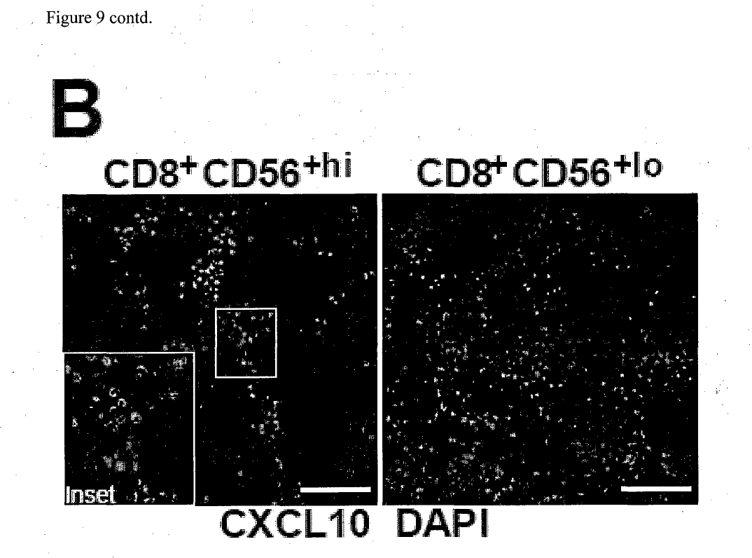

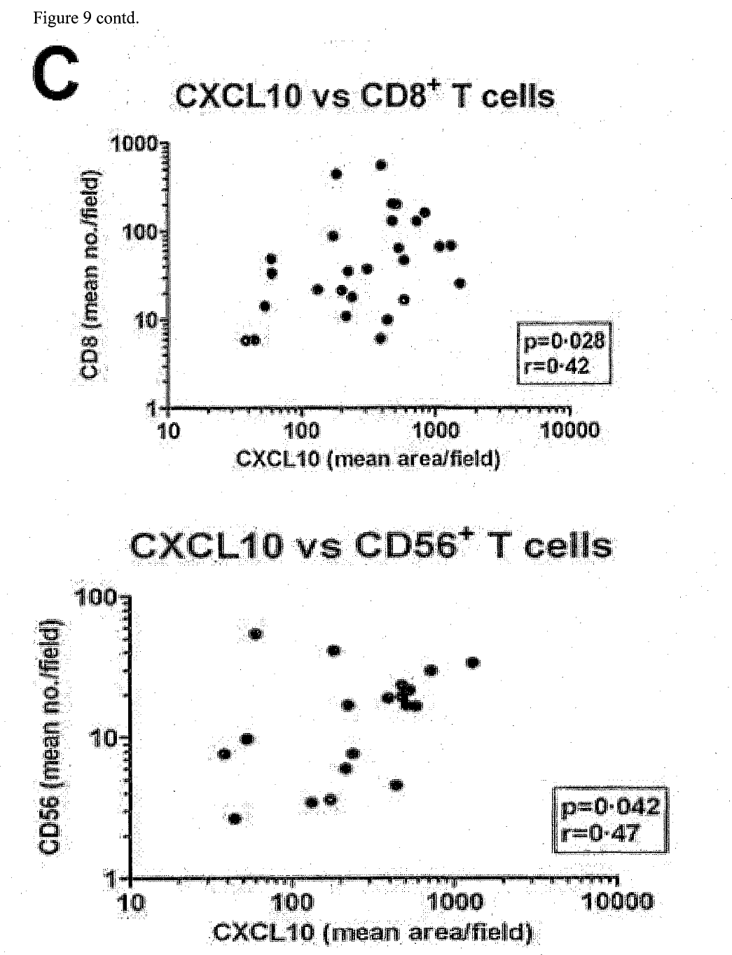

Chemokine and chemokine receptor genes such as CXCL10, CCL5, CCL2 and CCR2 constitute a prominent group in the immune signature identified. Since chemokines are critical for attracting immune cells [19], it was predicted that expression of these chemokines would correlate with tumor infiltration by defined immune cell subsets. To investigate this, correlations were searched for at the transcriptional level in 172 patient samples from both the training and validation cohorts. RNA expression of CXCL10, CCL5 and CCL2 correlated with markers of Th1 cells (TBX21), CD8⁺ T (CD8A) and NK (NCR3) cells (FIG. 9A). Interestingly, TBX21, CD8A and NCR3 are also among the genes present in the signature. There was no correlation between expression of these chemokines and markers of other immune cell subsets such as macrophages (CD14 and CD68), Th2 (IL13), Th17 (IL17), Treg (FoxP3 and IL10), B (CD19), or dendritic (CD83) cells (FIG. 9A). This shows that CXCL10, CCL5 and CCL2 are associated with, and likely to specifically attract, Th1, CD8⁺ T and NK cells into HCC tumors.

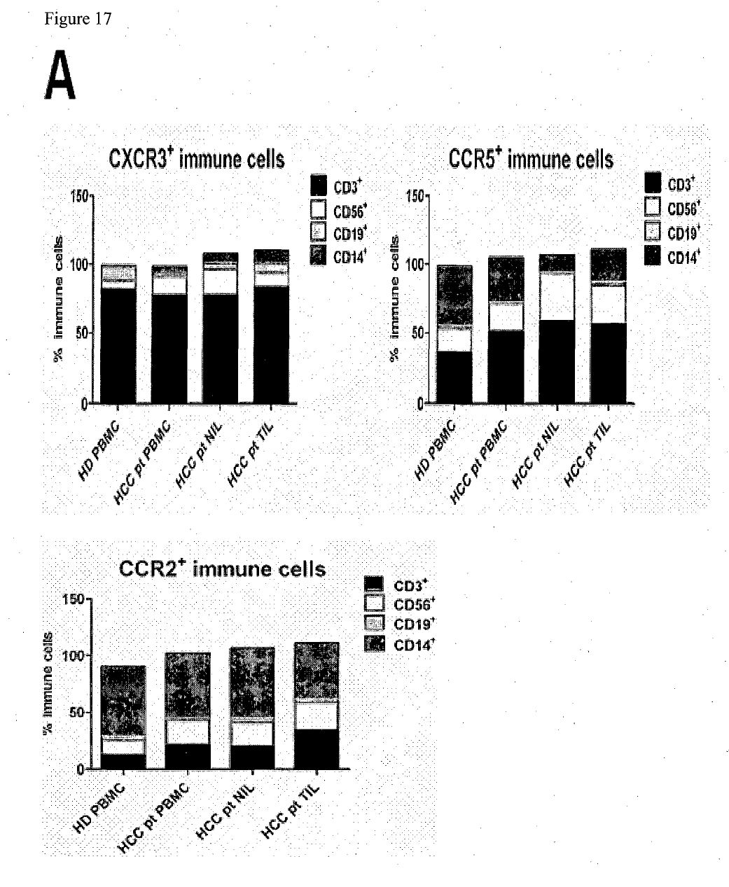

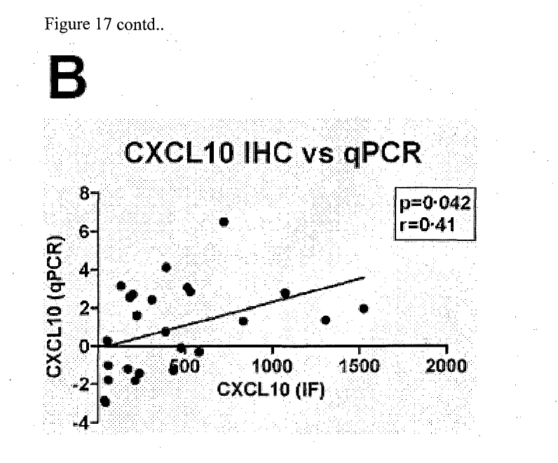

To further support this, the surface expression of CXCR3, CCR5 and CCR2 (the main receptors for CXCL10, CCL5 and CCL2 respectively) on peripheral blood mononuclear cells (PBMC) from healthy donors and HCC patients was measured, as well as on infiltrating leukocytes isolated from freshly-resected tumors (Tumor-infiltrating leukocytes or TIL) or adjacent non-tumoral tissues (Non-tumor-infiltrating lymphocytes or NIL). Flow cytometry analysis showed that T and NK cells represent the majority of the immune subsets expressing CXCR3 and CCR5 (FIG. 17A). Furthermore, a greater percentage of T and NK cells express CCR5 and CCR2 in patients PBMC, TIL and NIL as compared to healthy donor PBMC (FIG. 17A). This observation may indicate an increased propensity of T and NK cells from HCC patients to be attracted by CCL5 and CCL2.

CXCL10 expression in tumor sections using immunofluorescence was also analyzed. It was first verified that CXCL10-specific immunofluorescence correlated with mRNA expression (FIG. 17B). Next it was showed that higher CXCL10-specific immunofluorescence (FIG. 17B) was observed in samples with a higher density of CD8⁺ and CD56⁺ cells, as determined by IHC. Further quantification showed that the CXCL10 immunofluorescence correlated with the density of CD8⁺ T cells and CD56⁺ NK cells (CD8: n=27, p=0.028, r=0.42 and CD56: n=19, p=0.042, r=0.47) (FIG. 9C) and also with patient survival (n=25, p=0.024, hazard ratio=3.5) (FIG. 17C).

Taken together, these data strongly suggest that CXCL10, CCL5 and CCL2 are the main chemokines attracting Th1 T cells, CD8⁺ T cells and NK cells into the tumor microenvironment.

Chemokines Associated with Patient Survival are Produced by Both Cancer Cells and TIL

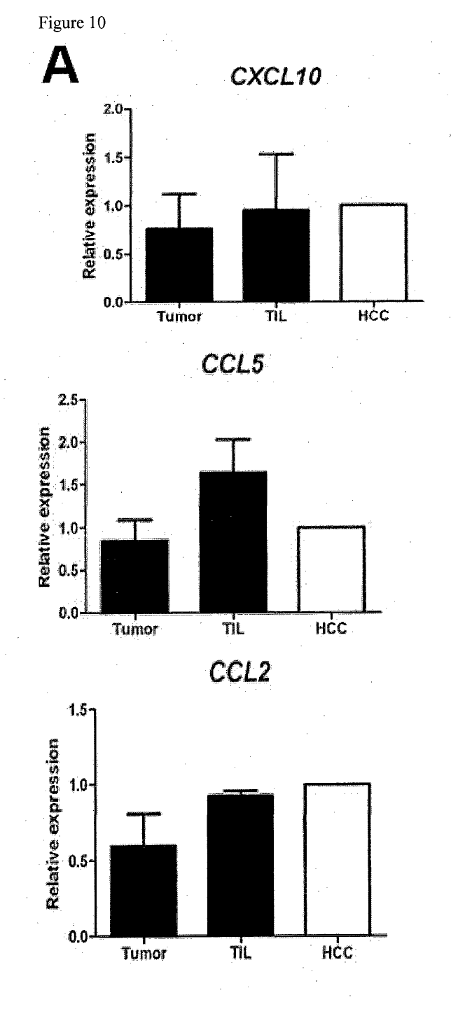

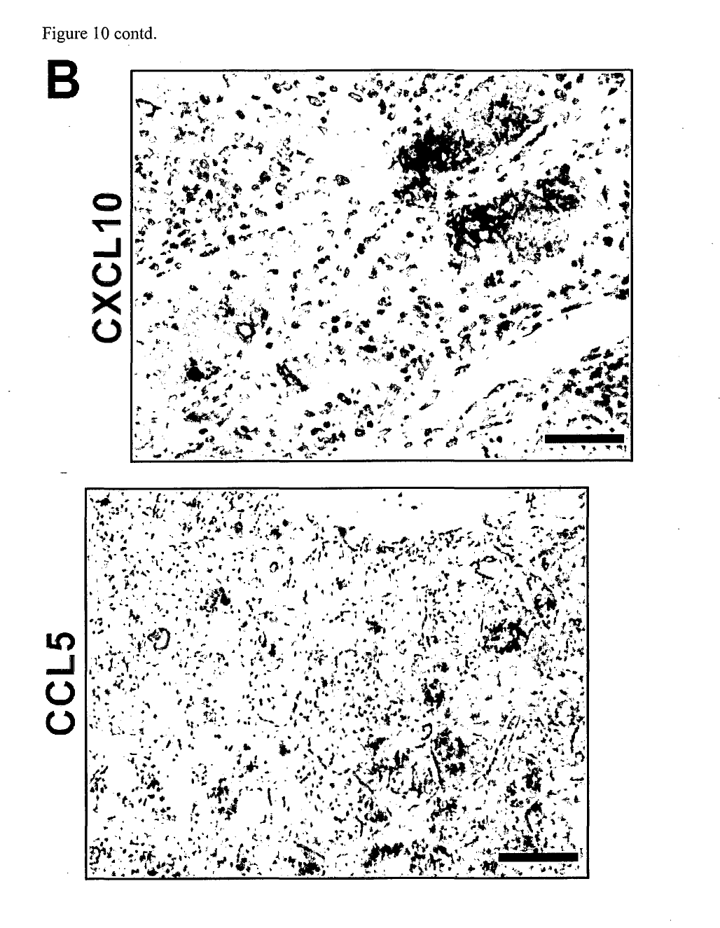

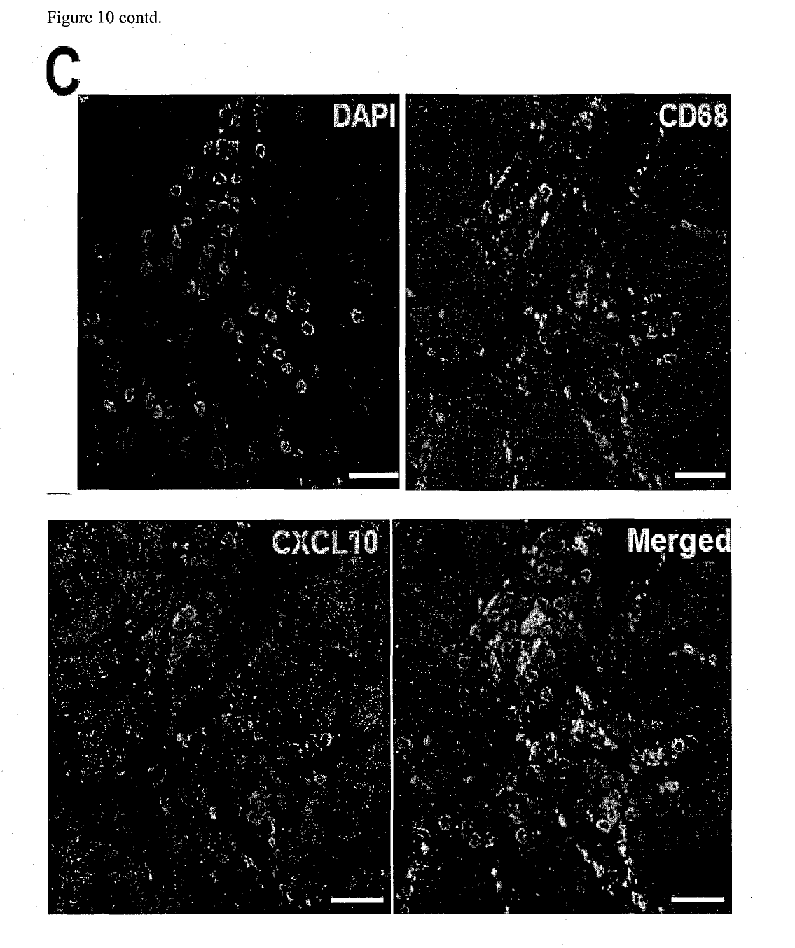

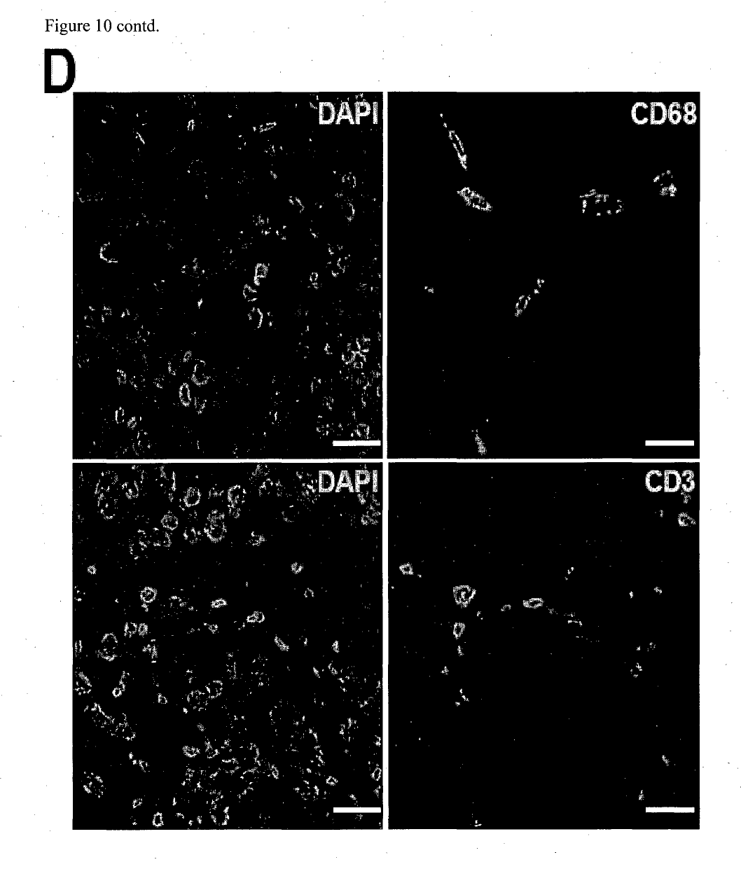

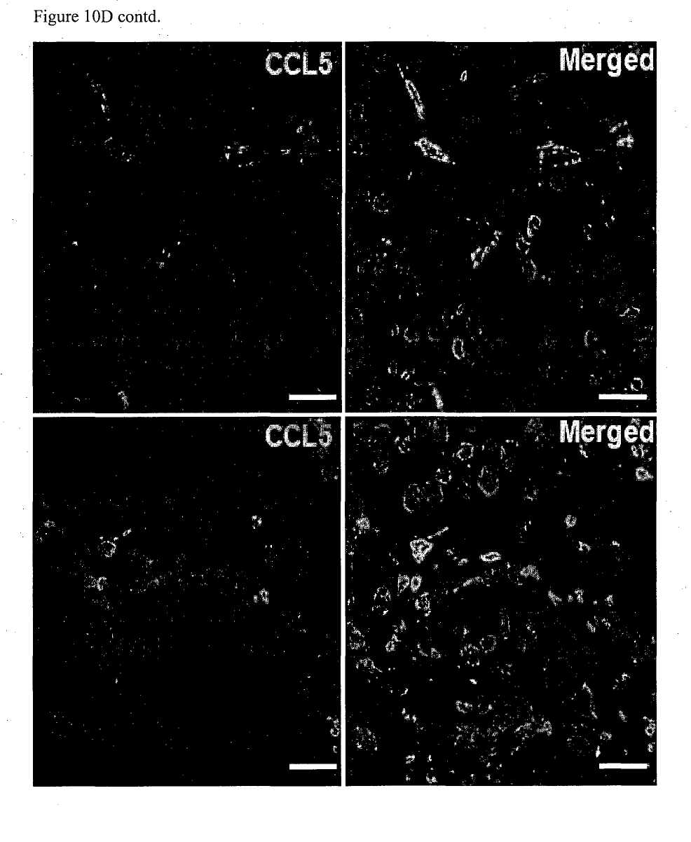

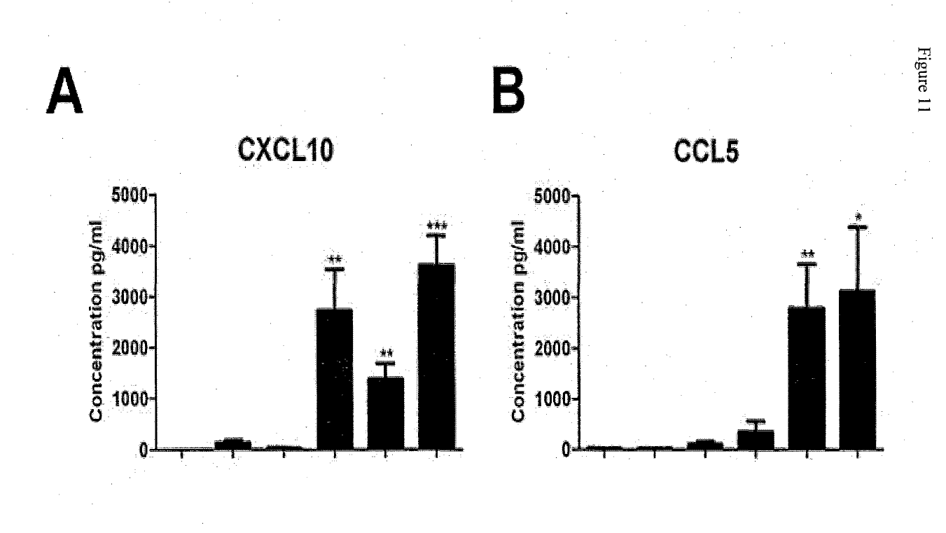

To understand the molecular interactions taking place within the tumor, the identity of the source of CXCL10, CCL5 and CCL2 within HCC was sought. Single cell suspensions from fresh tumor samples were separated into tumor cells and TIL, followed by chemokine expression analysis using qPCR. The three chemokine genes were transcribed in both tumor cells and TIL (FIG. 10, A). Furthermore, when CXCL10 and CCL5 expression was analyzed in situ by immunohistochemistry, many chemokine-producing cells exhibited cancer cell morphology (FIG. 10B). CXCL10 was also expressed by TIL. Immunofluorescence on tumor sections, combining labeling for CXCL10 and immune cell markers (CD68, CD3 and CD20) revealed that most of the CXCL10-producing immune cells co-expressed CD68 (FIG. 10C) but not T or B cell markers (data not shown). Similarly, co-localization of CCL5 and CD68 (FIG. 10D) were found. Hence, macrophages within HCC tumors express both CXCL10 and CCL5.

Besides macrophages, CCL5 was also produced by CD3⁺ T cells (FIG. 10D). Given the ability of CCL5 to attract T cells, this suggests an autocrine loop in which CCL5 produced by macrophages and/or cancer cells attracts T cells, which produce more CCL5 to further amplify T cell infiltration.

### TNF-α, IFN-γ and TLR3 Ligands Induce Expression of CXCL10, CCL5 and CCL2 by HCC Cells and Induce Transmigration of T and NK Cells.

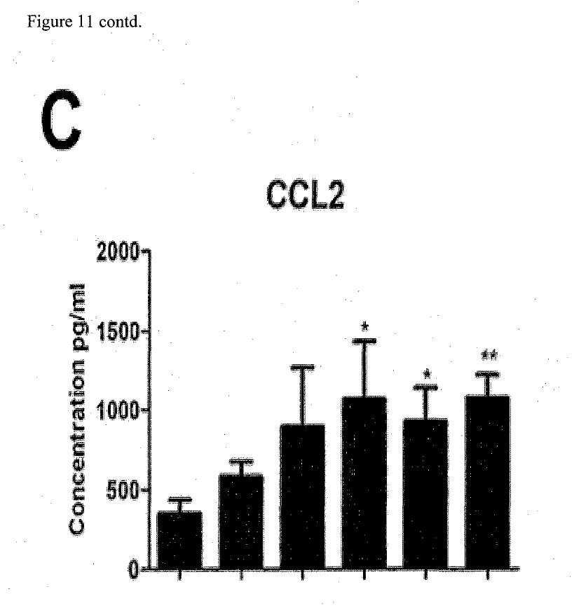

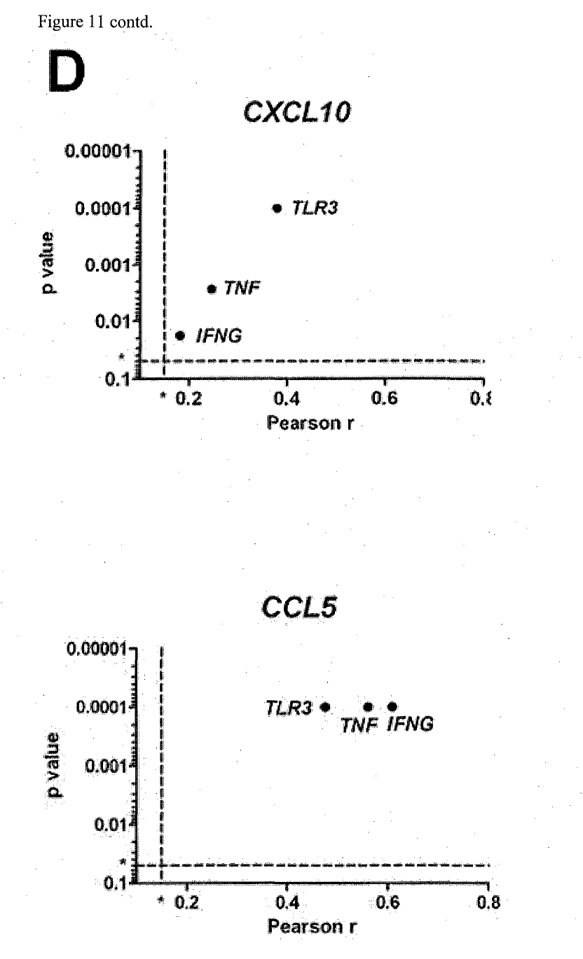

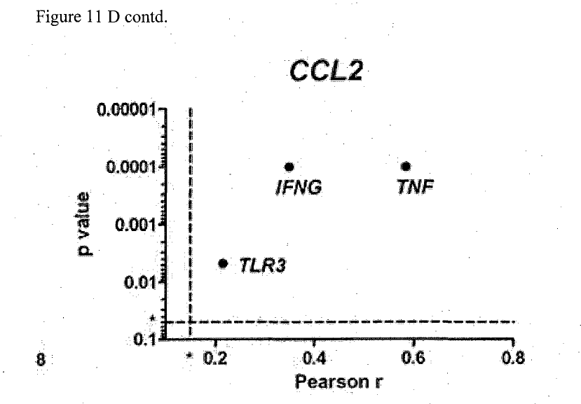

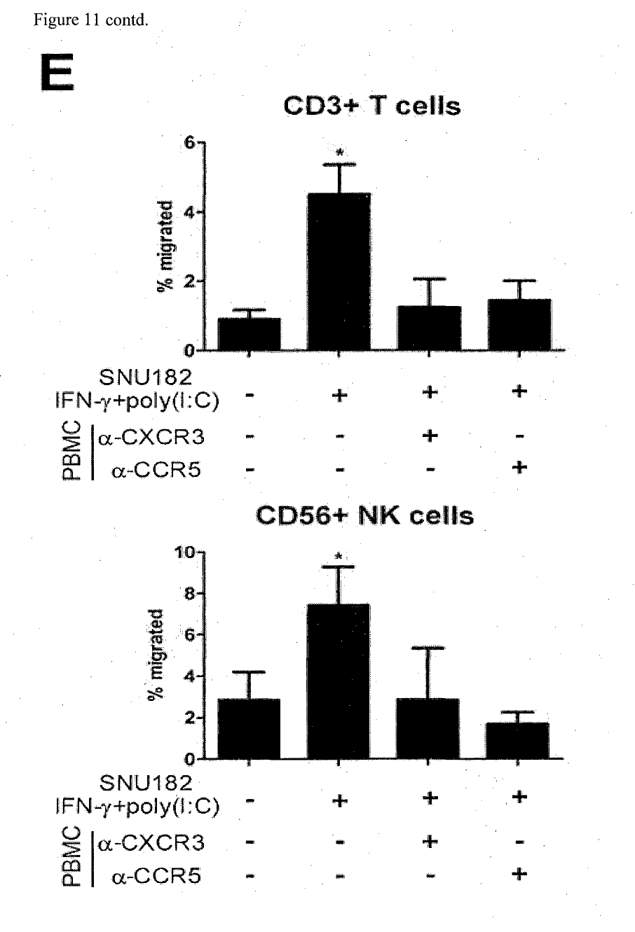

TNF-α, IFN-γ and TLR agonists stimulate CXCL10, CCL2 and CCL5 secretion by monocytes/macrophages [20-22], but little is known of the regulation of these chemokines in cancer cells. The HCC cell line SNU-182 was used to address this question. SNU-182 cells were treated with IFN-γ, TNF-α and the TLR3 ligand poly(I:C) separately or in combination, and culture supernatants were analyzed. While IFN-γ or TNF-α alone had little effect, CXCL10 was strongly induced by the combination of IFN-γ and TNF-α (FIG. 11A). Poly(I:C) alone significantly induced CXCL10 expression and this effect was further enhanced by addition of IFN-α (FIG. 11A). Poly(I:C) also induced CCL5 expression, while IFN-γ or TNF-α alone or in combination had no detectable effect (FIG. 11B). All three factors induced CCL2 expression but no synergistic effect was observed (FIG. 11C). Chemokine genes induction could be observed by qPCR already 6 hr after treatment (data not shown).

To validate these observations in patient samples, RNA expressions of CXCL10, CCL5 and CCL2 and those of IFNG, TNF and TLR3 within tumors were compared. Expression of the three chemokines correlated with those of IFNG, TNF and TLR3 (n=172 patients from both the training and validation cohorts; FIG. 11D).

Transwell migration assay was performed using stimulated SNU182 cells and healthy donor PBMC. The induction of chemokines in stimulated SNU182 cells induced transmigration of T (5 folds increase) & NK cells (2.5 folds increase), without affecting other leukocytes (data not shown). Transmigration of T and NK cells was abolished when PBMC were pretreated with anti-CXCR3 (CXCL10) and anti-CCR5 (CCL5) neutralizing antibodies (FIG. 11E).

Taken together, these data indicate that IFN-γ, TNF-α and TLR3 ligands are potent inducers of the survival-associated chemokines CXCL10, CCL5 and CCL2. These chemokines attract T and NK cells which, upon activation, produce more IFN-γ triggering a paracrine loop leading to further amplification of chemokine production and lymphocyte infiltration.

Lymphocyte-Attracting Chemokines are Associated with Enhanced Cancer Cell Death

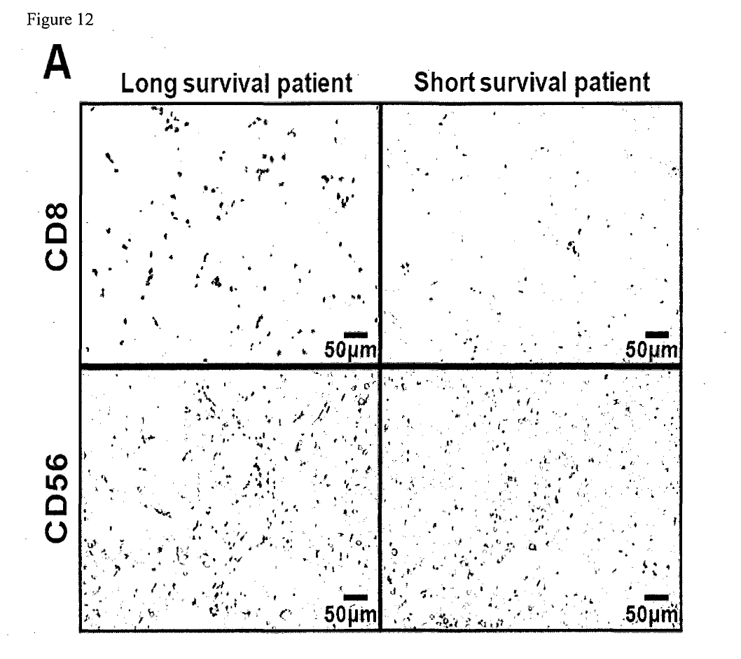

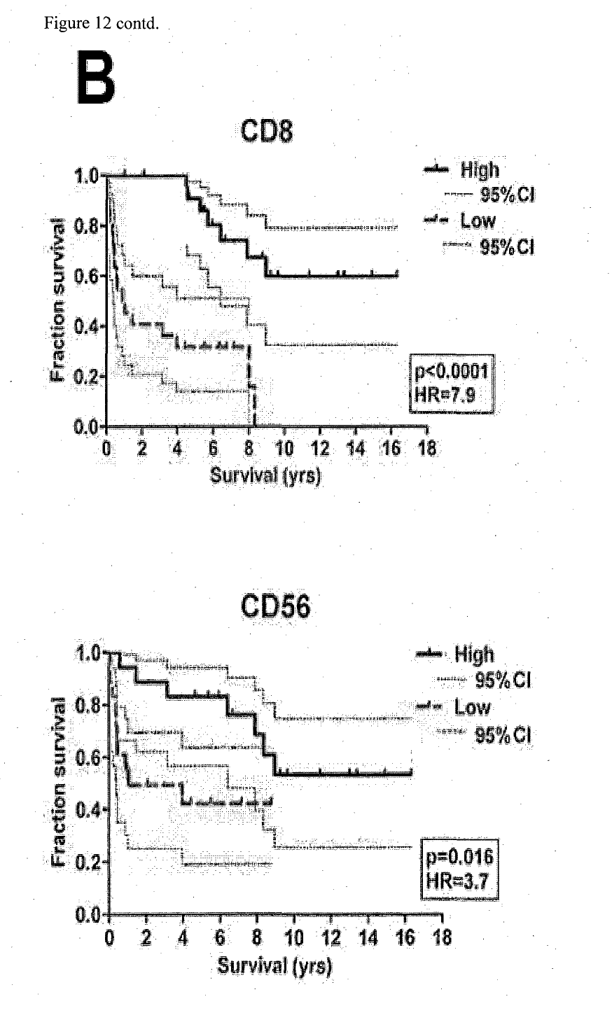

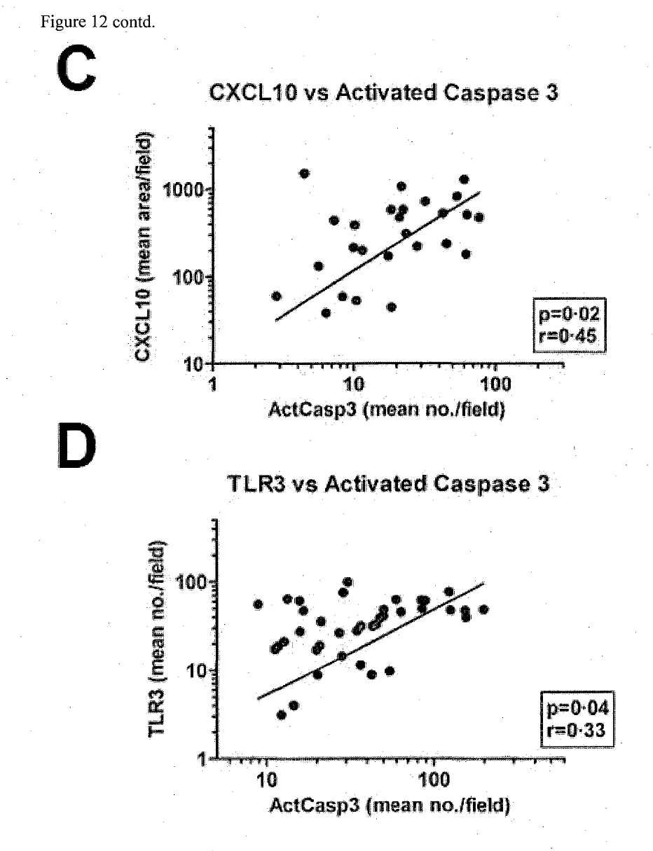

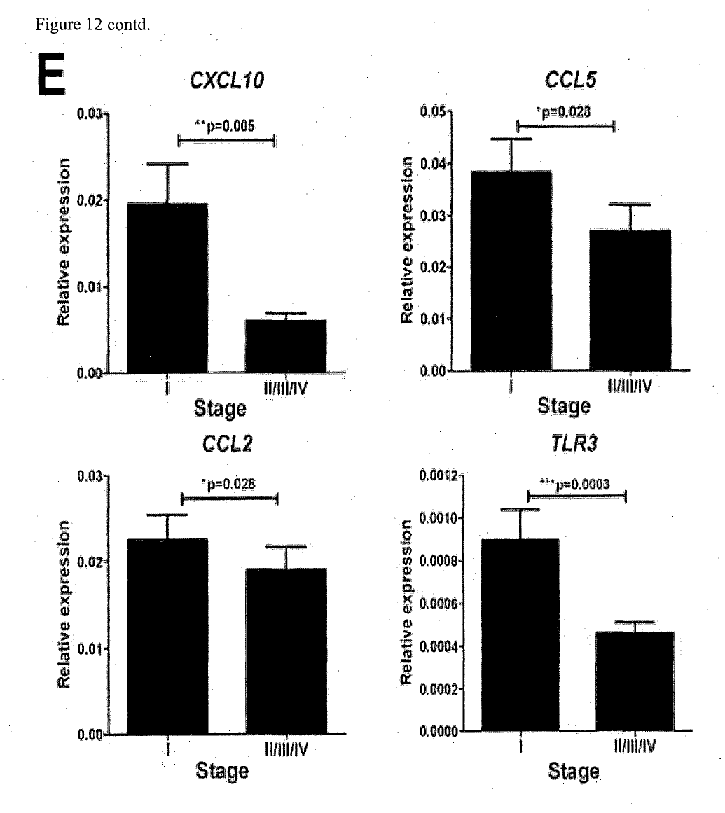

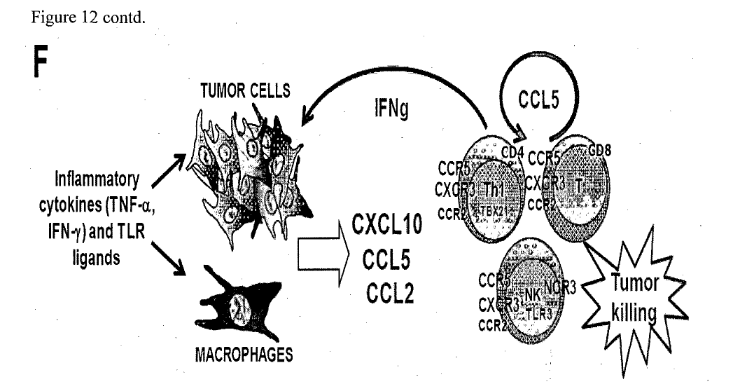

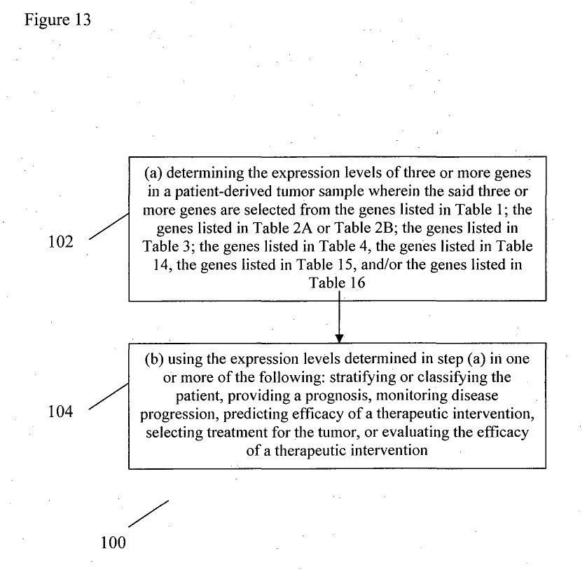

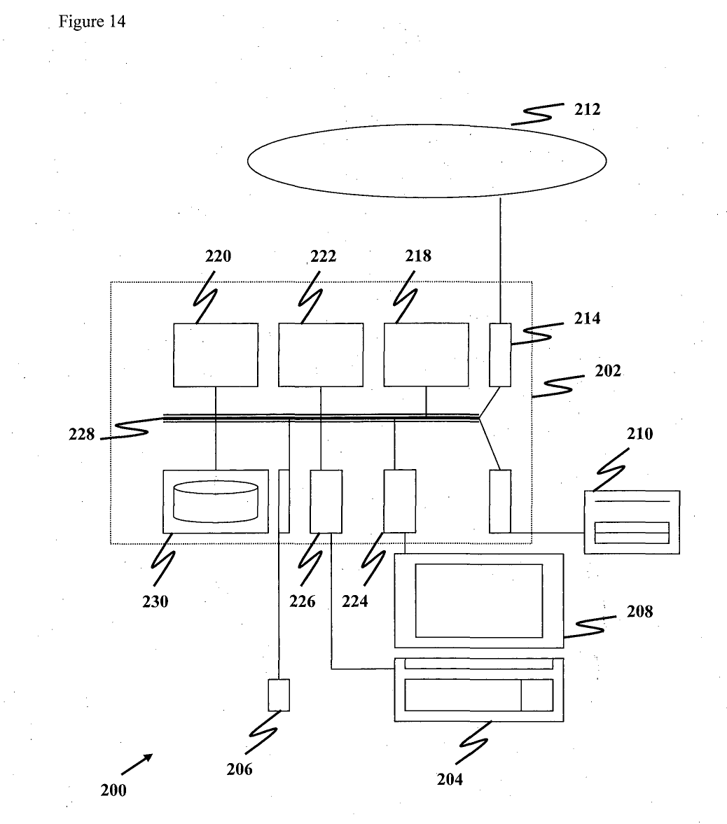

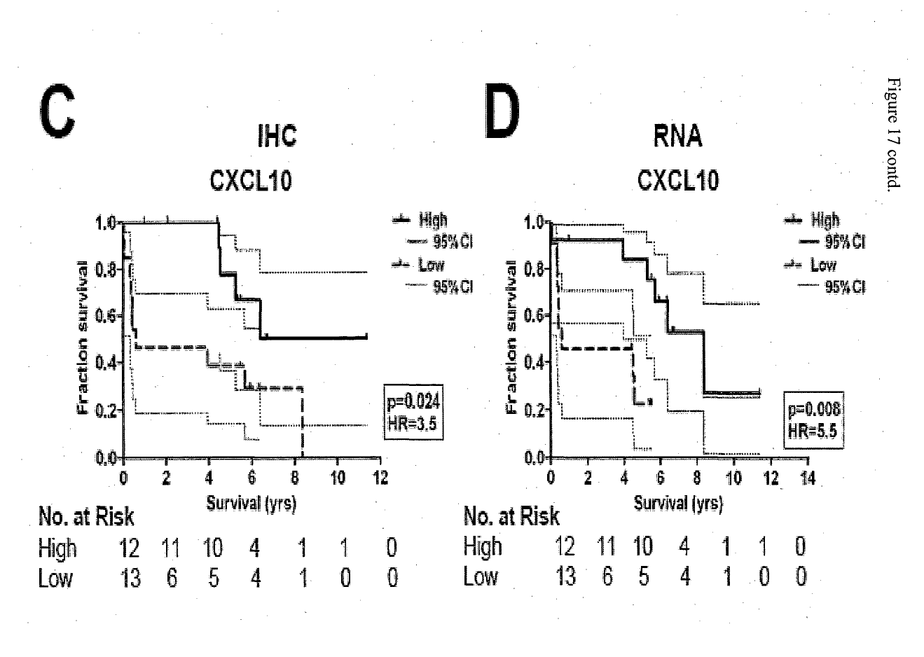

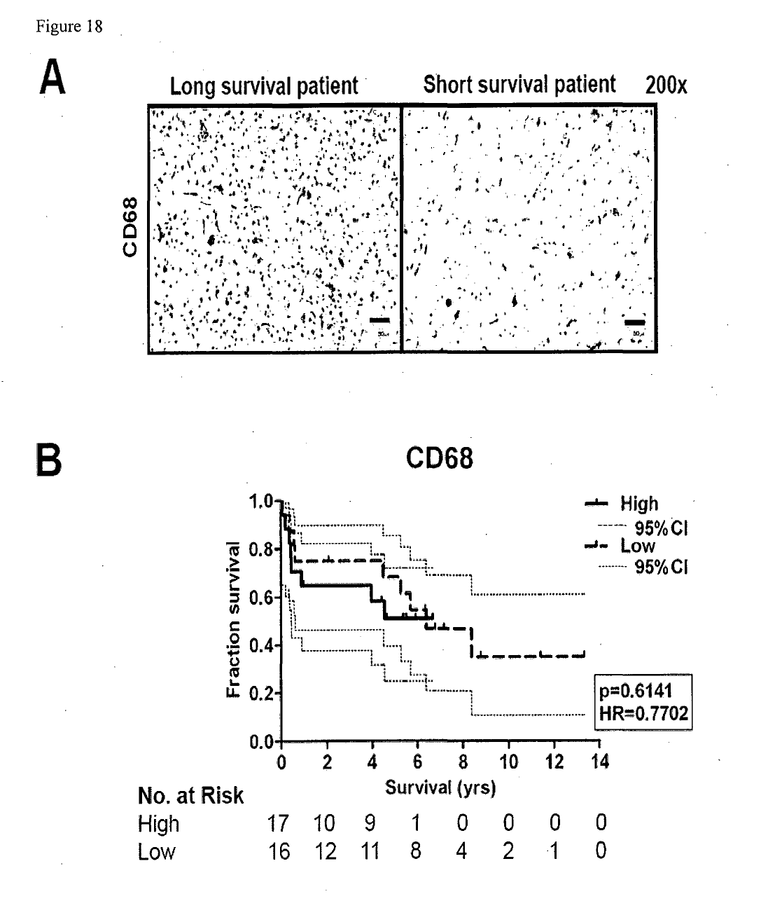

CD8A and NCR3, two genes specific for CD8+ T cells and NK cells respectively, are present in the gene signature and globally more expressed in long survivors. This is indeed reflected by enhanced infiltration of CD8⁺ T and CD56⁺ NK cells within the tumor samples from patients with longer survival (FIG. 12A, a subset of patients chosen for validation n=36 or 46). Kaplan-Meier analyses showed that a higher density of infiltrating CD8⁺ T (n=46, p<0.0001, hazard ratio=7.9) and CD56⁺ NK cells (n=36, p=0.016, hazard ratio=3.7) correlated with patient survival (FIG. 12B). Importantly, this was not observed for CD68⁺ macrophages (FIG. 18). In this subset of patients, the current immune signature was superior at predicting patient survival than tumor infiltration by T cells or NK cells.

It has previously been reported that the density of CD8⁺ T cells and CD56⁺ NK cells in HCC tumors correlates with cancer cell apoptosis detected by activated caspase-3 staining [16]. Since CXCL10 and TLR3 activation play a major role in recruiting these cells, it was examined if CXCL10 and TLR3 expressions correlate with cancer cell apoptosis. Indeed, protein expression of CXCL10 (n=26, p=0.02, r=0.45; FIG. 12C) and TLR3 (n=39, p=0.04, r=0.33; FIG. 12D), an important inducer of CXCL10, CCL5 and CCL2, correlated with activated caspase-3 expression in cancer cells. Taken together these correlations suggest a model in which chemokines expressed by cancer cells recruit lymphocytes that kill cancer cells, thereby contributing to prolonged patient survival. Such a model would predict that during the course of disease progression, cancer cells with reduced chemokines and TLR3 expression will be selected. Indeed, tumors from patients with more advances HCC (stage II to IV; n=114) exhibit significantly lower RNA expression of CXCL10, CCL5, CCL2 and TLR3 than those from stage I patients (n=57) (FIG. 12E). This further confirms the crucial role of chemokines in shaping a protective immune environment early in disease development.

### Discussion

In the present study an immune signature which predicts the survival in resectable HCC irrespective of patient ethnicity or etiology was identified. Interestingly, it predicts the survival of early stage patients for whom classical clinical parameters provide limited or no survival information. This signature, derived from resected HCC, comprises 14 genes coding for chemokines, inflammatory cytokines and lymphocyte markers. By combining transcriptome analysis, in situ staining and in vitro experiments, regulatory circuits that shape and maintain a protective immune milieu within the tumor, leading to prolonged patient survival were identified (FIG. 12F).

The immune signature was derived and tested using Singapore patients and further validated in an independent cohort from Hong Kong and Zurich. The predictive value of the signature was also verified separately in various subgroups of patients (FIG. 8E). This consistency across different subsets of patients indicates that immune parameters determining disease progression are conserved irrespective of HCC heterogeneity. This is remarkable since HCC is known to be derived from multiple cell types (including hepatocytes or adult stem/progenitor cells) and caused by several etiologies. Therefore, molecular features derived from the intratumoral immune response may be of better prognostic value than those relying on cancer cell characteristics. The loss of predictive power in female patients might be explained by the known gender disparity in the risk for HCC which is linked to estrogen-mediated inhibition of IL-6 [24-25] as IL-6 is one of the genes in the signature.

Previously, several studies using genomic approaches identified gene signatures that stratify HCC patients according to clinical prognosis [8-12]. These signatures were either derived from the adjacent non-tumor tissue or from the tumor itself. Signatures derived from the adjacent tissues emphasize on risk factors for developing de novo tumors and support the “field defect” hypothesis [10]. Interestingly, immune characteristics of the adjacent liver tissues have also been shown to impact patient survival [9-10]. On the other hand, signatures derived from the tumor itself focus on genes involved in proliferation and cell cycle [8, 11, 26] or on the identity of tumor-initiating cells [27-28]. The current study is the first to focus exclusively on immune genes expressed within the tumor, and to show that the HCC immune milieu has an impact on disease outcome.

It may seem paradoxical that inflammation, an established risk factor for developing HCC, could play a protective role in HCC progression [29-30]. For instance, IL-6 and TNF-α were shown to promote HCC tumorigenesis [31-33]. However, it was found that these two cytokines correlate with longer patient survival in the present study. The beneficial impact of an active immune response within the tumor microenvironment is well established for NSCLC [34], colorectal cancer [35-36] and other malignancies [37]. IL-6 and IL-8 were also reported to have a protective role in human colon adenomas [38]. Similarly, depending on the mouse model, NF-κB, a major regulator of inflammation, suppresses or promotes HCC development [39-40]. Additionally, expression of the same biomarker, for example IL-6, in the serum or within the tumor may also reflect different biological processes [16, 41]. These apparent contradictions indicate that the effect of inflammation is context-dependent and that the same cytokine may have opposite effects on HCC tumorigenesis and progression [42].

In the model, inflammatory cytokines (TNF-α and IFN-γ) and TLR ligands (likely released from necrotic cells) induce chemokine expression within the tumor microenvironment. These chemokines (CXCL10, CCL5 and CCL2) could recruit immune cells, which display anti-tumor activity reflected by enhanced activated caspase-3 expression in cancer cells. Furthermore, infiltrating immune cells augment chemokine production (possibly through secretion of IFN-γ or TNF-α upon activation [43]) or directly secrete chemokines (CCL5), further stabilizing the protective immune microenvironment. Such paracrine or autocrine loops are typical of complex biological systems as they provide efficient ways of amplifying signals and maintaining a particular immune status [44]. Interestingly, no single cell type or molecular cue plays a unique role in shaping the immune microenvironment. Chemokines are produced by both cancer cells and TIL, while IFN-γ is produced by Th1 and NK cells. Such redundancy also participates in the robustness of the protective environment, which has to be maintained for years in order to impact patient survival. The current immune signature predicts survival in stage I and II but not in stage III and IV patients. This shows that a protective immune response has to be established early enough to be effective. Hence it is proposed that once the tumor has been established for prolonged periods of time, multiple layers of immune tolerance may prevent the efficacy of anti-tumor responses [45-46]. It was therefore predictable and also shown in this study that cancer progression would be associated with down-regulation of chemokines critically involved in the shaping of a protective immune microenvironment.

In summary, this study reveals extensive crosstalk between cancer cells and tumor-infiltrating immune cells in establishing a protective immune milieu able to delay HCC progression. Improved understanding of the molecular pathways leading to a protective immune microenvironment will help in the rational design of new therapeutic approaches for HCC patients.

### REFERENCES

1 El-Serag H B. Epidemiology of hepatocellular carcinoma in USA. Hepatol Res 2007; 37 SUPPL 2:S88-94.
2 Parkin D M, Bray F, Ferlay J, et al. Global cancer statistics, 2002. CA Cancer J Clin 2005; 55:74-108.
3 Siegel A B, Olsen S K, Magun A, et al. Sorafenib: where do we go from here? Hepatology 2010; 52:360-9.
4 Llovet J M, Burroughs A, Bruix J. Hepatocellular carcinoma. Lancet 2003; 362:1907-17.
5 Hoshida Y, Nijman S M, Kobayashi M, et al. Integrative transcriptome analysis reveals common molecular subclasses of human hepatocellular carcinoma. Cancer Res 2009; 69:7385-92.
6 Zucman-Rossi J. Molecular classification of hepatocellular carcinoma. Dig Liver Dis 2010; 42 Suppl 3:S235-41.
7 Schutte K, Bornschein J, Malfertheiner P. Hepatocellular carcinoma—epidemiological trends and risk factors. Dig Dis 2009; 27:80-92.
8 Boyault S, Rickman D S, de Reynies A, et al. Transcriptome classification of HCC is related to gene alterations and to new therapeutic targets. Hepatology 2007; 45:42-52.
9 Budhu A, Forgues M, Ye Q H, et al. Prediction of venous metastases, recurrence, and prognosis in hepatocellular carcinoma based on a unique immune response signature of the liver microenvironment. Cancer Cell 2006; 10:99-111.
10 Hoshida Y, Villanueva A, Kobayashi M, et al. Gene expression in fixed tissues and outcome in hepatocellular carcinoma. N Engl J Med 2008; 359:1995-2004.
11 Lee J S, Chu I S, Heo J, et al. Classification and prediction of survival in hepatocellular carcinoma by gene expression profiling. Hepatology 2004; 40:667-76.
12 Ye Q H, Qin L X, Forgues M, et al. Predicting hepatitis B virus-positive metastatic hepatocellular carcinomas using gene expression profiling and supervised machine learning. Nat Med 2003; 9:416-23.
13 Allavena P, Sica A, Solinas G, et al. The inflammatory micro-environment in tumor progression: the role of tumor-associated macrophages. Crit Rev Oncol Hematol 2008; 66:1-9.
14 Sica A, Larghi P, Mancino A, et al. Macrophage polarization in tumour progression. Semin Cancer Biol 2008; 18:349-55.
15 Pages F, Galon J, Dieu-Nosjean M C, et al. Immune infiltration in human tumors: a prognostic factor that should not be ignored. Oncogene 2010; 29:1093-102.
16 Chew V, Tow C, Teo M, et al. Inflammatory tumour microenvironment is associated with superior survival in hepatocellular carcinoma patients. J Hepatol 2010; 52:370-9.
17 Henderson A R. The bootstrap: a technique for data-driven statistics. Using computer-intensive analyses to explore experimental data. Clin Chim Acta 2005; 359:1-26.
18 Hoshida Y. Nearest template prediction: a single-sample-based flexible class prediction with confidence assessment. PLoS One 2010; 5:e15543.
19 Shurin M R, Shurin G V, Lokshin A, et al. Intratumoral cytokines/chemokines/growth factors and tumor infiltrating dendritic cells: friends or enemies? Cancer Metastasis Rev 2006; 25:333-56.
20 Bauermeister K, Burger M, Almanasreh N, et al. Distinct regulation of IL-8 and MCP-1 by LPS and interferon-gamma-treated human peritoneal macrophages. Nephrol Dial Transplant 1998; 13:1412-9.
21 Marfaing-Koka A, Maravic M, Humbert M, et al. Contrasting effects of IL-4, IL-10 and corticosteroids on RANTES production by human monocytes. Int Immunol 1996; 8:1587-94.
22 Qi X F, Kim D H, Yoon Y S, et al. Essential involvement of cross-talk between IFN-gamma and TNF-alpha in CXCL10 production in human THP-1 monocytes. J Cell Physiol 2009; 220:690-7.
23 Lee J S, Heo J, Libbrecht L, et al. A novel prognostic subtype of human hepatocellular carcinoma derived from hepatic progenitor cells. Nat Med 2006; 12:410-6.
24 Naugler W E, Sakurai T, Kim S, et al. Gender disparity in liver cancer due to sex differences in MyD88-dependent IL-6 production. Science 2007; 317:121-4.
25 Prieto J. Inflammation, HCC and sex: IL-6 in the centre of the triangle. J Hepatol 2008; 48:380-1.
26 Chiang D Y, Villanueva A, Hoshida Y, et al. Focal gains of VEGFA and molecular classification of hepatocellular carcinoma. Cancer Res 2008; 68:6779-88.
27 Andersen J B, Loi R, Perra A, et al. Progenitor-derived hepatocellular carcinoma model in the rat. Hepatology 2010; 51:1401-9.
28 Yamashita T, Ji J, Budhu A, et al. EpCAM-positive hepatocellular carcinoma cells are tumor-initiating cells with stem/progenitor cell features. Gastroenterology 2009; 136:1012-24.
29 Marotta F, Vangieri B, Cecere A, et al. The pathogenesis of hepatocellular carcinoma is multifactorial event. Novel immunological treatment in prospect. Clin Ter 2004; 155:187-99.
30 Matsuzaki K, Murata M, Yoshida K, et al. Chronic inflammation associated with hepatitis C virus infection perturbs hepatic transforming growth factor beta signaling, promoting cirrhosis and hepatocellular carcinoma. Hepatology 2007; 46:48-57.
31 He G, Karin M. NF-kappaB and STAT3-key players in liver inflammation and cancer. Cell Res 2011; 21:159-68.
32 Wong V W, Yu J, Cheng A S, et al. High serum interleukin-6 level predicts future hepatocellular carcinoma development in patients with chronic hepatitis B. Int J Cancer 2009; 124:2766-70.
33 Wu J M, Xu Y, Skill N J, et al. Autotaxin expression and its connection with the TNF-alpha-NF-kappaB axis in human hepatocellular carcinoma. Mol Cancer 2010; 9:71.
34 Dieu-Nosjean M C, Antoine M, Danel C, et al. Long-term survival for patients with non-small-cell lung cancer with intratumoral lymphoid structures. J Clin Oncol 2008; 26:4410-7.
35 Ohtani H. Focus on TILs: prognostic significance of tumor infiltrating lymphocytes in human colorectal cancer. Cancer Immun 2007; 7:4.
36 Galon J, Costes A, Sanchez-Cabo F, et al. Type, density, and location of immune cells within human colorectal tumors predict clinical outcome. Science 2006; 313:1960-4.
37 Zitvogel L, Apetoh L, Ghiringhelli F, et al. The anticancer immune response: indispensable for therapeutic success? J Clin Invest 2008; 118:1991-2001.
38 Kuilman T, Michaloglou C, Vredeveld L C, et al. Oncogene-induced senescence relayed by an interleukin-dependent inflammatory network. Cell 2008; 133:1019-31.
39 Maeda S, Kamata H, Luo J L, et al. IKKbeta couples hepatocyte death to cytokine-driven compensatory proliferation that promotes chemical hepatocarcinogenesis. Cell 2005; 121:977-90.
40 Pikarsky E, Porat R M, Stein I, et al. NF-kappaB functions as a tumour promoter in inflammation-associated cancer. Nature 2004; 431:461-6.
41 Chau G Y, Wu C W, Lui W Y, et al. Serum interleukin-10 but not interleukin-6 is related to clinical outcome in patients with resectable hepatocellular carcinoma. Ann Surg 2000; 231:552-8.
42 de Visser K E, Eichten A, Coussens L M. Paradoxical roles of the immune system during cancer development. Nat Rev Cancer 2006; 6:24-37.
43 Doherty D G, Norris S, Madrigal-Estebas L, et al. The human liver contains multiple populations of NK cells, T cells, and CD3+ CD56+ natural T cells with distinct cytotoxic activities and Th1, Th2, and Th0 cytokine secretion patterns. J Immunol 1999; 163:2314-21.
44 Kitano H. Biological robustness. Nat Rev Genet 2004; 5:826-37.
45 Bergmann C, Strauss L, Wang Y, et al. T regulatory type 1 cells in squamous cell carcinoma of the head and neck: mechanisms of suppression and expansion in advanced disease. Clin Cancer Res 2008; 14:3706-15.
46 Zitvogel L, Tesniere A, Kroemer G. Cancer despite immunosurveillance: immunoselection and immunosubversion. Nat Rev Immunol 2006; 6:715-27.

1.-26. (canceled)

27. A method of analysing a patient with Hepatocellular Carcinoma (HCC), wherein the method comprises:
(a) determining the expression levels of three or more genes in a patient-derived tumor sample wherein the said three or more genes are selected from the genes listed in Table 1; the genes listed in Table 2A or Table 2B; the genes listed in Table 3; the genes listed in Table 4, the genes listed in Table 14, the genes listed in Table 15, and/or the genes listed in Table 16; and
(b) using the expression levels determined in step (a) in one or more of the following: stratifying or classifying the patient, providing a prognosis, monitoring disease progression, predicting efficacy of a therapeutic intervention, selecting treatment for the tumor, or evaluating the efficacy of a therapeutic intervention.

28. The method according to claim 27 wherein the method is a method of classifying a patient with HCC as having a poor or good prognosis comprising the steps of:
(a) determining the expression levels of three or more genes (and preferably five or more genes) in a patient-derived tumor sample, wherein the genes are selected from the genes listed in Table 1; the genes listed in Table 2A or Table 2B; the genes listed in Table 3; the genes listed in Table 4, the genes listed in Table 14, the genes listed in Table 15, and/or the genes listed in Table 16; and
(b) classifying the patient as having a short or long survival based on the expression levels determined in step (a), in which the patient has HCC.

29. The method according to claim 27 wherein the method is a method for evaluating the efficacy of a therapeutic intervention for treating HCC patients comprising the steps of:
(a) determining the expression levels of three or more genes in a patient-derived tumor sample, wherein the genes are selected from the genes listed in Table 1; the genes listed in Table 2; the genes listed in Table 3; the genes listed in Table 4, the genes listed in Table 14, the genes listed in Table 15, and/or the genes listed in Table 16; and
(b) classifying the patient as having a short or long survival based on the expression levels determined in step (a), in which the patient has HCC, and in which classification of a patient by step (b) is monitored before, during and/or after the therapeutic intervention.

30. The method according to claim 27 in which:
(i) the three or more genes of Table 1 are at least 3, 4, 5, 6, 7, 8, 9, 10, 11, 12, 13, 14, and/or all the genes listed in Table 1 and/or any combination thereof;
(ii) the three or more genes of Table 2A are at least 3, 4, 5, 6 and/or all of the genes listed in Table 2A and/or any combination thereof;
(iii) the three or more genes of Table 2B are at least 3, 4, 5, 6 and/or all of the genes listed in Table 2B and/or any combination thereof;
(iv) the three or more genes of Table 3 are at least 3, 4, 5, 6, 7, 8, 9, 10 and/or all of the genes listed in Table 3 and/or any combination thereof;
(v) the three or more genes of Table 4 are at least 3, 4, 5, 6, 7, 8, 9, 10, 11, 12, 13 and/or all of the genes listed in Table 4 and/or any combination thereof;
(vi) the three or more genes of Table 14 are at least 3, 4, 5, 6, 7 and/or all of the genes listed in Table 14 and/or any combination thereof;
(vii) the three or more genes of Table 15 are at least 3, 4 and/or all of the genes listed in Table 15 and/or any combination thereof;
(viii) the three or more genes of Table 16 are at least 3, 4 and/or all of the genes listed in Table 16 and/or any combination thereof;
(ix) wherein 14 genes are selected from Table 1;
(x) the three or more genes of Table 1 are between 4 to 15 genes, 4 to 14 genes, 5 to 15 genes, or 5 to 14 genes from Table 1; or
(xi) the three or more genes of Table 1 comprise: CCL2, CCL5 and CCR2; CCL5, CCL2 and CXCL10; or CCL5, CCL2, CXCL10 and CCR2.

31. The method according to claim 28 in which:
(i) the three or more genes of Table 1 are at least 3, 4, 5, 6, 7, 8, 9, 10, 11, 12, 13, 14, and/or all the genes listed in Table 1 and/or any combination thereof;
(ii) the three or more genes of Table 2A are at least 3, 4, 5, 6 and/or all of the genes listed in Table 2A and/or any combination thereof;
(iii) the three or more genes of Table 2B are at least 3, 4, 5, 6 and/or all of the genes listed in Table 2B and/or any combination thereof;
(iv) the three or more genes of Table 3 are at least 3, 4, 5, 6, 7, 8, 9, 10 and/or all of the genes listed in Table 3 and/or any combination thereof;
(v) the three or more genes of Table 4 are at least 3, 4, 5, 6, 7, 8, 9, 10, 11, 12, 13 and/or all of the genes listed in Table 4 and/or any combination thereof;
(vi) the three or more genes of Table 14 are at least 3, 4, 5, 6, 7 and/or all of the genes listed in Table 14 and/or any combination thereof;
(vii) the three or more genes of Table 15 are at least 3, 4 and/or all of the genes listed in Table 15 and/or any combination thereof;
(viii) the three or more genes of Table 16 are at least 3, 4 and/or all of the genes listed in Table 16 and/or any combination thereof;
(ix) wherein 14 genes are selected from Table 1;
(x) the three or more genes of Table 1 are between 4 to 15 genes, 4 to 14 genes, 5 to 15 genes, or 5 to 14 genes from Table 1; or
(xi) the three or more genes of Table 1 comprise: CCL2, CCL5 and CCR2; CCL5, CCL2 and CXCL10; or CCL5, CCL2, CXCL10 and CCR2.

32. The method according to claim 29 in which:
(i) the three or more genes of Table 1 are at least 3, 4, 5, 6, 7, 8, 9, 10, 11, 12, 13, 14, and/or all the genes listed in Table 1 and/or any combination thereof;
(ii) the three or more genes of Table 2A are at least 3, 4, 5, 6 and/or all of the genes listed in Table 2A and/or any combination thereof;
(iii) the three or more genes of Table 2B are at least 3, 4, 5, 6 and/or all of the genes listed in Table 2B and/or any combination thereof;
(iv) the three or more genes of Table 3 are at least 3, 4, 5, 6, 7, 8, 9, 10 and/or all of the genes listed in Table 3 and/or any combination thereof;
(v) the three or more genes of Table 4 are at least 3, 4, 5, 6, 7, 8, 9, 10, 11, 12, 13 and/or all of the genes listed in Table 4 and/or any combination thereof;
(vi) the three or more genes of Table 14 are at least 3, 4, 5, 6, 7 and/or all of the genes listed in Table 14 and/or any combination thereof;
(vii) the three or more genes of Table 15 are at least 3, 4 and/or all of the genes listed in Table 15 and/or any combination thereof;
(viii) the three or more genes of Table 16 are at least 3, 4 and/or all of the genes listed in Table 16 and/or any combination thereof;
(ix) wherein 14 genes are selected from Table 1;
(x) the three or more genes of Table 1 are between 4 to 15 genes, 4 to 14 genes, 5 to 15 genes, or 5 to 14 genes from Table 1; or
(xi) the three or more genes of Table 1 comprise: CCL2, CCL5 and CCR2; CCL5, CCL2 and CXCL10; or CCL5, CCL2, CXCL10 and CCR2.

33. The method according to claim 27 wherein step (b) uses additional information in stratifying or classifying the patient, providing a prognosis, monitoring disease progression, predicting efficacy of a therapeutic intervention, selecting treatment for the tumor, or evaluating the efficacy of a therapeutic intervention, and wherein such additional information is optionally staging information and/or the expression (present or absent, or the level of) of one or more further marker genes which are not found in Table 1, 2A, 2B, 3, 4, 14, 15 or 16 and which said one or more further marker genes is of predictive value for HCC prognosis.

34. The method according to claim 27 wherein one or more of the following apply: (a) the expression levels are normalized expression levels and/or relative expression levels; (b) the patient-derived tumor sample comprises tumor infiltrating leukocytes (TIL), stroma and tumor cells; (c) the patient is human.

35. The method according to claim 27 wherein step (b) comprises deriving a value from the expression levels of the three or more genes listed in Table 1, 2A, 2B, 3, 4, 14, 15, or 16 (and optionally also from the expression levels of any one or more further marker genes which may be employed) and comparing the value with a threshold value wherein a determination that the derived value is below or above said threshold value indicates a particular prognosis (e.g. a good or poor prognosis),
and optionally wherein: (i) a poor prognosis is less than 3, 4, 5 or 6 years predicted survival and a good prognosis is more than or equal to 3, 4, 5 or 6 years predicted survival; or (ii) a poor prognosis is less than the median survival years of a given cohort and a good prognosis is more than the median survival years of a given cohort.

36. The method according to claim 27 wherein an expression profile comprises the expression levels of said three or more genes listed in Table 1, 2A, 2B, 3, 4, 14, 15, or 16 and wherein step (b) comprises determining the similarity of the expression profile to a good prognosis template and/or a poor prognosis template, wherein the degree of similarity to the good prognosis template and/or poor prognosis template indicates whether the patient has a good prognosis or poor prognosis.

37. The method according to claim 36 wherein step (b) comprises determining the similarity of the expression profile to a good prognosis template and/or a poor prognosis template, and wherein said patient is classified as having: (i) a good prognosis if said expression profile is similar to the good prognosis template and/or is dissimilar to the poor prognosis template; or (ii) a poor prognosis if said expression profile is dissimilar to the good prognosis template and/or is similar to the poor prognosis template, wherein the expression profile is determined as being similar or dissimilar to the template depending on whether the similarity is above or below a predetermined threshold value.

38. The method according to claim 36 wherein step (b) comprises determining the similarity of the expression profile to a good prognosis template and/or a poor prognosis template, and wherein said patient is classified as having: (i) a good prognosis if said expression profile has a higher similarity to said good prognosis template than to said poor prognosis template; or (ii) a poor prognosis if said expression profile has a higher similarity to said poor prognosis template than to said good prognosis template.

39. The method according to claim 27 in which step (b) is performed using at least one algorithm, and/or a computer.

40. The method according to claim 39 in which step (b) is performed using a SVM algorithm, a KNN algorithm or a combination of an SVM and a KNN algorithm, and optionally wherein:
(i) the three or more genes of Table 2A are at least 3, 4, 5, 6 and/or all of the genes listed in Table 2A and/or any combination thereof; or the three or more genes of Table 2B are at least 3, 4, 5, 6 and/or all of the genes listed in Table 2B and/or any combination thereof; or the three or more genes of Table 1 are at least 3, 4, 5, 6, 7, 8, 9, 10, 11, 12, 13, 14 and/or all of the genes listed in Table 1 and/or any combination thereof; or the three or more genes of Table 14 are at least 3, 4, 5, 6, 7 and/or all of the genes listed in Table 14; or the three or more genes of Table 15 are at least 3, 4 and/or all of the genes listed in Table 15; or the three or more genes of Table 16 are at least 3, 4 and/or all of the genes listed in Table 16, and step (b) is performed using the SVM algorithm; or
(ii) the three or more genes of Table 3 are at least 3, 4, 5, 6, 7, 8, 9, 10 and/or all of the genes listed in Table 3 and/or any combination thereof; or the three or more genes of Table 1 are at least 3, 4, 5, 6, 7, 8, 9, 10, 11, 12, 13, 14 and/or all of the genes listed in Table 1 and/or any combination thereof, and wherein step (b) is performed using the KNN algorithm.

41. The method according to claim 39 in which step (b) is performed using an NTP algorithm, and optionally wherein the three or more genes of Table 4 are at least 3, 4, 5, 6, 7, 8, 9, 10, 11, 12, 13 and/or all of the genes listed in Table 4 and/or any combination thereof or the three or more genes of Table 1 are at least 3, 4, 5, 6, 7, 8, 9, 10, 11, 12, 13, 14 and/or all of the genes listed in Table 1 and/or any combination thereof.

42. The method according to claim 39, wherein step (b) is performed by: (i) application of an SVM algorithm as described in Algorithm 1, classifies a patient as having a good or poor prognosis; or (ii) application of an KNN algorithm as described in Algorithm 2, classifies a patient as having a good or poor prognosis.

43. The method according to claim 39, wherein step (b) is performed by application of an NTP algorithm as described in Algorithm 3, classifies a patient as having a good or poor prognosis.

44. The method according to claim 27 wherein the method is a method for evaluating the efficacy of a therapeutic intervention for treating HCC patients and wherein the therapeutic intervention is a candidate agent.

45. The method according to claim 27 further comprising selecting the patient for therapy or follow-up on the basis of the patient having either a good or poor prognosis.

46. The method according to claim 27 wherein said therapeutic intervention is a neoadjuvant treatment.

47. The method according to claim 27 comprising use of a microarray kit or quantitative PCR to determine the expression level of any or all of the genes listed in Table 1, Table 2A, Table 2B, Table 3, Table 4, Table 14, Table 15 or Table 16.

48. The method according to claim 27 wherein the HCC is stage I or stage II.

49. A method of treating a patient characterised as a patient having either good or poor prognosis according to claim 27, wherein said patient is administered with a hepatocellular-carcinoma immunotherapy or any other alternative treatments.

50. A kit for use in claim 27, wherein the kit comprises reagents for determining the expression of said three or more genes selected from the genes listed in Table 1, the genes listed in Table 2A or Table 2B, the genes listed in Table 3, the genes listed in Table 4, the genes listed in Table 14, the genes listed in Table 15 and/or the genes listed in Table 16 and wherein the kit further optionally comprises instructions for use.

51. A computer program or computer software product for performing step (b) of a method according to claim 27, or a computer system programmed to perform step (b) of a method according to claim 27.

52. A microarray for use in a method according to claim 27, wherein the microarray comprises a plurality of probes capable of hybridizing to the said three or more genes selected from the genes listed in Table 1, the genes listed in Table 2A or Table 2B, the genes listed in Table 3, the genes listed in Table 4, the genes listed in Table 14, the genes listed in Table 15, and/or the genes listed in Table 14.

53. A method of providing an HCC human patient with a good or a poor prognosis, wherein the method comprises:
(a) determining the expression levels of five or more genes in a tumor sample derived from said patient, which tumor sample comprises total tumor material, wherein the said five or more genes are selected from at least one list of genes selected from the group consisting of the genes listed in Table 1; the genes listed in Table 2A; the genes listed in Table 2B; the genes listed in Table 3; the genes listed in Table 4, the genes listed in Table 14, the genes listed in Table 15, and the genes listed in Table 16, and wherein the expression levels may optionally be relative expression levels and/or normalized expression levels; and
(b) determining the similarity of an expression profile comprising the expression levels determined in step (a) to a good prognosis template which comprises gene expression levels characteristic of good prognosis patients and a poor prognosis template which comprises gene expression levels characteristic of poor prognosis patient, wherein a higher similarity of said expression profile to said good prognosis template indicates a poor prognosis and a higher similarity to said poor prognosis template than to said good prognosis template indicates a poor prognosis,
and wherein a poor prognosis is less than the median survival years of a given cohort and a good prognosis is more than the median survival years of a given cohort.
